(19) United States
(12) Patent Application Publication
(10) Pub. No.: US 2010/0151495 A9
(43) Pub. Date: Jun. 17, 2010

(54) Reagents for the detection of protein phosphorylation in carcinoma signaling pathways

(75) Inventors: Roberto Polakiewicz, Lexington, MA (US); Ailan Guo, Burlington, MA (US); Albrecht Moritz, Salem, MA (US); Klarisa Rikova, Reading, MA (US); Kimberly Lee, Seattle, WA (US); Erik Spek, Cambridge, MA (US); Yu Li, Andover, MA (US); Charles Farnsworth, Concord, MA (US)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(73) Assignee: CELL SIGNALING TECHNOLGY, INC.

(21) Appl. No.: 12/074,228

(22) Filed: Feb. 29, 2008

Related U.S. Application Data
(63) Continuation of application No. PCT/US06/34063, filed on Aug. 31, 2006.
(60) Provisional application No. 60/712,997, filed on Aug. 31, 2005.

(51) Int. Cl. G01N 33/53; C07K 16/18; C07K 14/00; C12N 5/18

(52) U.S. Cl. 435/7.8; 436/536; 530/387.7; 530/402; 435/346

(57) ABSTRACT

The invention discloses nearly 443 novel phosphorylation sites identified in signal transduction proteins and pathways underlying human carcinoma, and provides phosphorylation-site specific antibodies and heavy-isotope labeled peptides (AQUA peptides) for the selective detection and quantification of these phosphorylated sites/proteins, as well as methods of using the reagents for such purpose. Among the phosphorylation sites identified are sites occurring in the following protein types: Protein kinases (including Serine/Threonine dual specificity, and Tyrosine kinases), Adaptor/Scaffold proteins, Transcription factors, Phospoatases, Tumor supressors, Ubiquitin conjugating system proteins, Translation initiation complex proteins, RNA binding proteins, Apoptosis proteins, Adhesion proteins, G protein regulators/GTPase activating protein/Guanine nucleotide exchange factor proteins, and DNA binding/replication/repair proteins, as well as other protein types.

### RELATED APPLICATIONS

This application claims the benefit of, and priority to, PCT serial number PCT/US06/034063, filed Aug. 31, 2006, presently pending, the disclosure of which is incorporated herein, in its entirety, by reference.

### FIELD OF THE INVENTION

The invention relates generally to antibodies and peptide reagents for the detection of protein phosphorylation, and to protein phosphorylation in cancer.

### BACKGROUND OF THE INVENTION

The activation of proteins by post-translational modification is an important cellular mechanism for regulating most aspects of biological organization and control, including growth, development, homeostasis, and cellular communication. Protein phosphorylation, for example, plays a critical role in the etiology of many pathological conditions and diseases, including cancer, developmental disorders, autoimmune diseases, and diabetes. Yet, in spite of the importance of protein modification, it is not yet well understood at the molecular level, due to the extraordinary complexity of signaling pathways, and the slow development of technology necessary to unravel it.

Protein phosphorylation on a proteome-wide scale is extremely complex as a result of three factors: the large number of modifying proteins, e.g. kinases, encoded in the genome, the much larger number of sites on substrate proteins that are modified by these enzymes, and the dynamic nature of protein expression during growth, development, disease states, and aging. The human genome, for example, encodes over 520 different protein kinases, making them the most abundant class of enzymes known. See Hunter, Nature 411: 355-65 (2001). Most kinases phosphorylate many different substrate proteins, at distinct tyrosine, serine, and/or threonine residues. Indeed, it is estimated that one-third of all proteins encoded by the human genome are phosphorylated, and many are phosphorylated at multiple sites by different kinases.

Many of these phosphorylation sites regulate critical biological processes and may prove to be important diagnostic or therapeutic targets for molecular medicine. For example, of the more than 100 dominant oncogenes identified to date, 46 are protein kinases. See Hunter, supra. Understanding which proteins are modified by these kinases will greatly expand our understanding of the molecular mechanisms underlying oncogenic transformation. Therefore, the identification of, and ability to detect, phosphorylation sites on a wide variety of cellular proteins is crucially important to understanding the key signaling proteins and pathways implicated in the progression of diseases like cancer.

Carcinoma is one of the two main categories of cancer, and is generally characterized by the formation of malignant tumors or cells of epithelial tissue original, such as skin, digestive tract, glands, etc. Carcinomas are malignant by definition, and tend to metastasize to other areas of the body. The most common forms of carcinoma are skin cancer, lung cancer, breast cancer, and colon cancer, as well as other numerous but less prevalent carcinomas. Current estimates show that, collectively, various carcinomas will account for approximately 1.65 million cancer diagnoses in the United States alone, and more than 300,000 people will die from some type of carcinoma during 2005. (Source: American Cancer Society (2005)). The worldwide incidence of carcinoma is much higher.

As with many cancers, deregulation of receptor tyrosine kinases (RTKs) appears to be a central theme in the etiology of carcinomas. Constitutively active RTKs can contribute not only to unrestricted cell proliferation, but also to other important features of malignant tumors, such as evading apoptosis, the ability to promote blood vessel growth, the ability to invade other tissues and build metastases at distant sites (see Blume-Jensen et al., Nature 411: 355-365 (2001)). These effects are mediated not only through aberrant activity of RTKs themselves, but, in turn, by aberrant activity of their downstream signaling molecules and substrates.

The importance of RTKs in carcinoma progression has led to a very active search for pharmacological compounds that can inhibit RTK activity in tumor cells, and more recently to significant efforts aimed at identifying genetic mutations in RTKs that may occur in, and affect progression of, different types of carcinomas (see, e.g., Bardell et al., Science 300: 949 (2003); Lynch et al., N. Eng. J. Med. 350: 2129-2139 (2004)). For example, non-small cell lung carcinoma patients carrying activating mutations in the epidermal growth factor receptor (EGFR), an RTK, appear to respond better to specific EGFR inhibitors than do patients without such mutations (Lynch et al., supra.; Paez et al., Science 304:1497-1500 (2004)).

Clearly, identifying activated RTKs and downstream signaling molecules driving the oncogenic phenotype of carcinomas would be highly beneficial for understanding the underlying mechanisms of this prevalent form of cancer, identifying novel drug targets for the treatment of such disease, and for assessing appropriate patient treatment with selective kinase inhibitors of relevant targets when and if they become available.

However, although a few key RTKs involved in carcinoma progression are known, there is relatively scarce information about kinase-driven signaling pathways and phosphorylation sites that underly the different types of carcinoma. Therefore there is presently an incomplete and inaccurate understanding of how protein activation within signaling pathways is driving these complex cancers. Accordingly, there is a continuing and pressing need to unravel the molecular mechanisms of kinase-driven oncogenesis in carcinoma by identifying the downstream signaling proteins mediating cellular transformation in these cancers. Identifying particular phosphorylation sites on such signaling proteins and providing new reagents, such as phospho-specific antibodies and AQUA peptides, to detect and quantify them remains especially important to advancing our understanding of the biology of this disease.

Presently, diagnosis of carcinoma is made by tissue biopsy and detection of different cell surface markers. However, misdiagnosis can occur since some carcinoma cases can be negative for certain markers and because these markers may not indicate which genes or protein kinases may be deregulated. Although the genetic translocations and/or mutations characteristic of a particular form of carcinoma can be sometimes detected, it is clear that other downstream effectors of constitutively active kinases having potential diagnostic, predictive, or therapeutic value, remain to be elucidated. Accordingly, identification of downstream signaling molecules and phosphorylation sites involved in different types of carcinoma and development of new reagents to detect and quantify these sites and proteins may lead to improved diagnostic/prognostic markers, as well as novel drug targets, for the detection and treatment of this disease.

### SUMMARY OF THE INVENTION

The invention discloses nearly 443 novel phosphorylation sites identified in signal transduction proteins and pathways underlying human carcinomas and provides new reagents, including phosphorylation-site specific antibodies and AQUA peptides, for the selective detection and quantification of these phosphorylated sites/proteins. Also provided are methods of using the reagents of the invention for the detection, quantification, and profiling of the disclosed phosphorylation sites.

### BRIEF DESCRIPTION OF THE DRAWINGS

FIG. 1—Is a diagram broadly depicting the immunoaffinity isolation and mass-spectrometric characterization methodology (IAP) employed to identify the novel phosphorylation sites disclosed herein.

FIG. 2—Is a table (corresponding to Table 1) enumerating the 443 carcinoma signaling protein phosphorylation sites disclosed herein: Column A=the name of the parent protein; Column B=the SwissProt accession number for the protein (human sequence); Column C=the protein type/classification; Column D=the tyrosine residue (in the parent protein amino acid sequence) at which phosphorylation occurs within the phosphorylation site; Column E=the phosphorylation site sequence encompassing the phosphorylatable residue (residue at which phosphorylation occurs (and corresponding to the respective entry in Column D) appears in lowercase; Column F=the type of carcinoma in which the phosphorylation site was discovered; Column G=the cell type(s) in which the phosphorylation site was discovered; and Column H=the SEQ ID NO.

FIG. 3—is an exemplary mass spectrograph depicting the detection of the tyrosine 1048 phosphorylation site in flt 1 (see Row 164 in FIG. 2/Table 1), as further described in Example 1 (red and blue indicate ions detected in MS/MS spectrum); Y* (and pY) indicates the phosphorylated tyrosine (shown as lowercase “y” in FIG. 2).

FIG. 4—is an exemplary mass spectrograph depicting the detection of the tyrosine 2556 phosphorylation site in NF1 (see Row 128 in FIG. 2/Table 1), as further described in Example 1 (red and blue indicate ions detected in MS/MS spectrum); Y* (and pY) indicates the phosphorylated tyrosine (shown as lowercase “y” in FIG. 2).

FIG. 5—is an exemplary mass spectrograph depicting the detection of the tyrosine 315 phosphorylation site in OCLN (see Row 44 in FIG. 2/Table 1), as further described in Example 1 (red and blue indicate ions detected in MS/MS spectrum); Y* (and pY) indicates the phosphorylated tyrosine (shown as lowercase “y” in FIG. 2) and M# (and lowercase “m”) indicates an oxidized methionine also detected.

FIG. 6—is an exemplary mass spectrograph depicting the detection of the tyrosine 1200 phosphorylation site in PHLPP (see Row 193 in FIG. 2/Table 1), as further described in Example 1 (red and blue indicate ions detected in MS/MS spectrum); Y* (and pY) indicates the phosphorylated tyrosine (shown as lowercase “y” in FIG. 2).

FIG. 7—is an exemplary mass spectrograph depicting the detection of the tyrosine 366 phosphorylation site in TNS1 (see Row 20 in FIG. 2/Table 1), as further described in Example 1 (red and blue indicate ions detected in MS/MS spectrum); Y* (and pY) indicates the phosphorylated tyrosine (shown as lowercase “y” in FIG. 2).

FIG. 8—is an exemplary mass spectrograph depicting the detection of the tyrosine 188 phosphorylation site in Yap1 (see Row 328 in FIG. 2/Table 1), as further described in Example 1 (red and blue indicate ions detected in MS/MS spectrum); Y* (and pY) indicates the phosphorylated tyrosine (shown as lowercase “y” in FIG. 2).

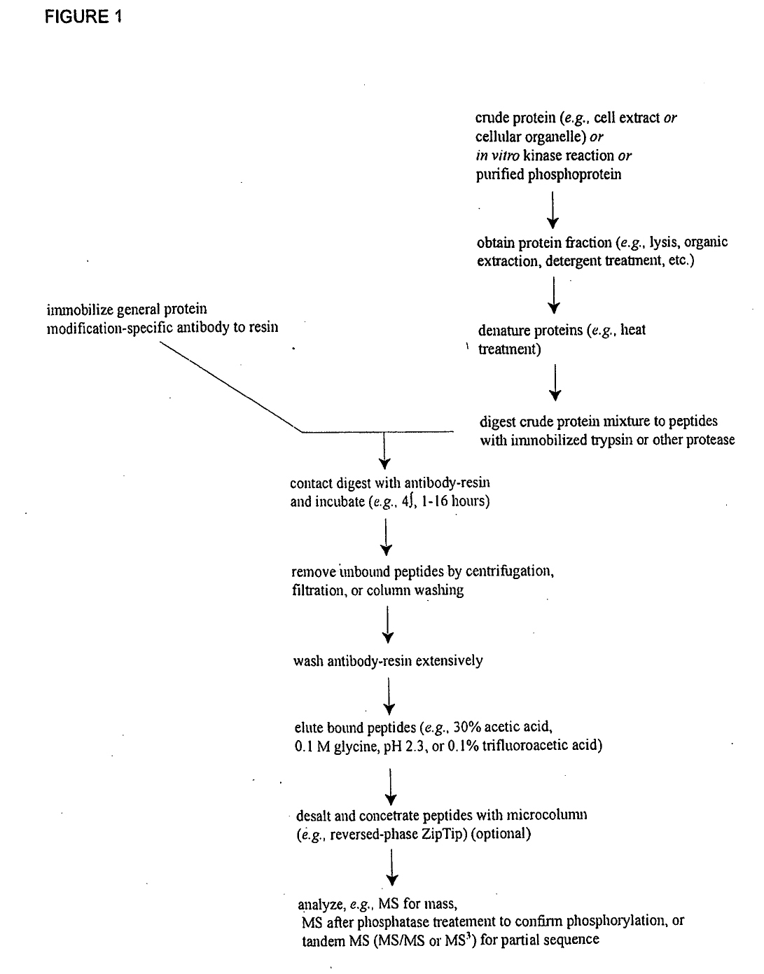

### DETAILED DESCRIPTION OF THE INVENTION

In accordance with the present invention, nearly 443 novel protein phosphorylation sites in signaling proteins and pathways underlying carcinoma have now been discovered. These newly described phosphorylation sites were identified by employing the techniques described in “Immunoaffinity Isolation of Modified Peptides From Complex Mixtures,” U.S. Patent Publication No. 20030044848, Rush et al., using cellular extracts from a variety of human carcinoma-derived cell lines, such as 3T3-abl, U118 MG, 293T, NCI-N87, A549, etc., as further described below. The novel phosphorylation sites (tyrosine), and their corresponding parent proteins, disclosed herein are listed in Table 1.

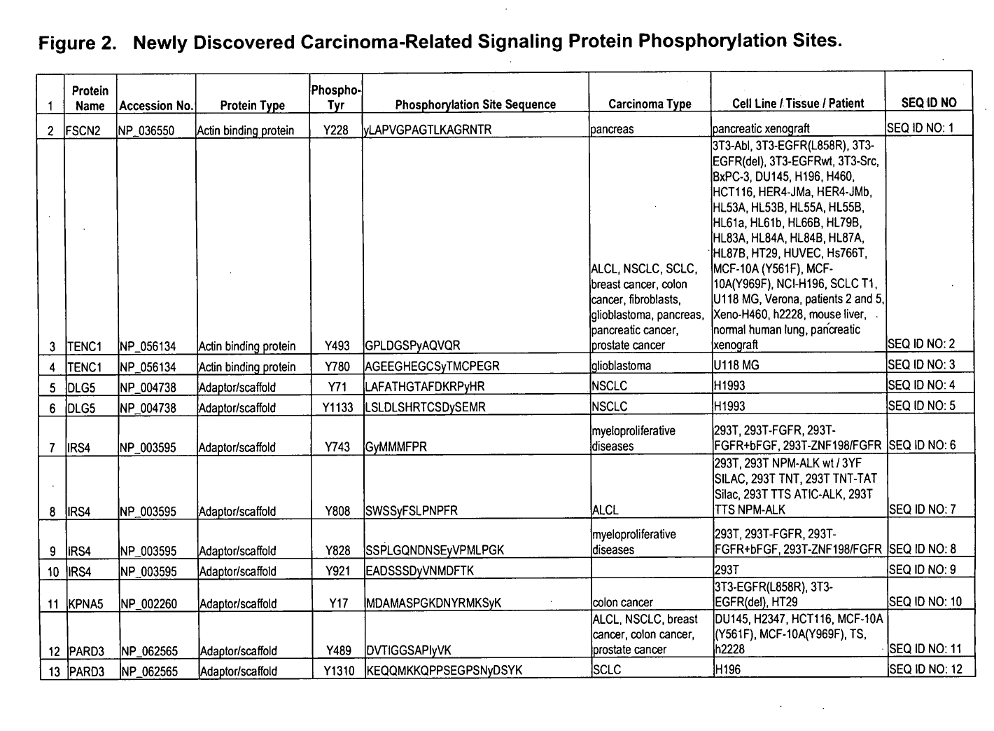

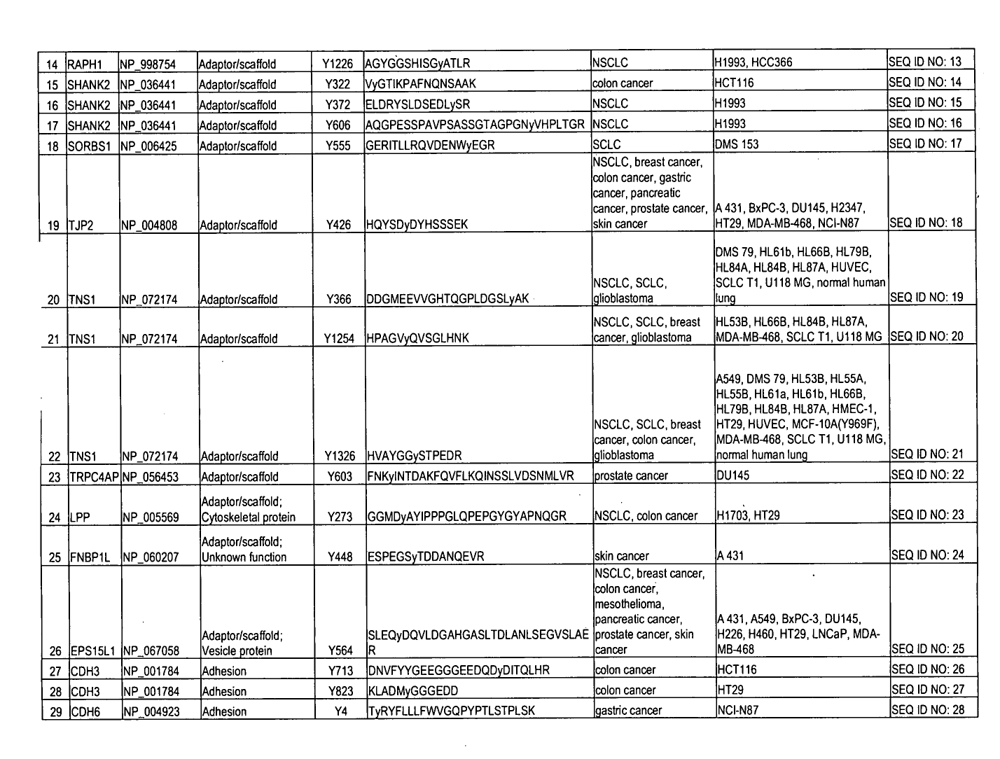

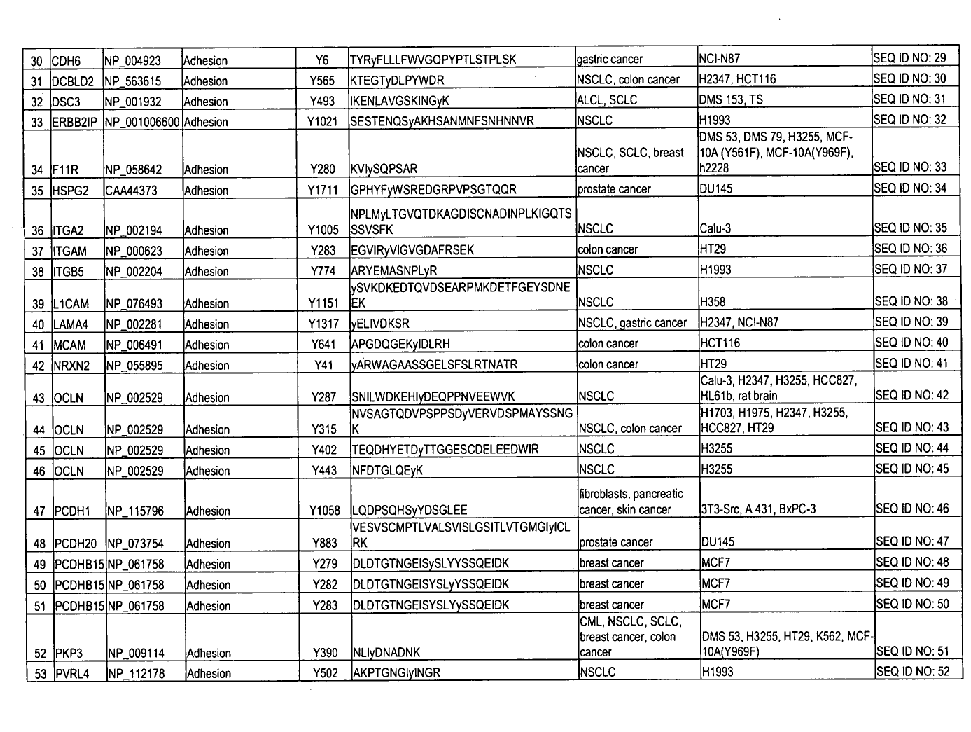

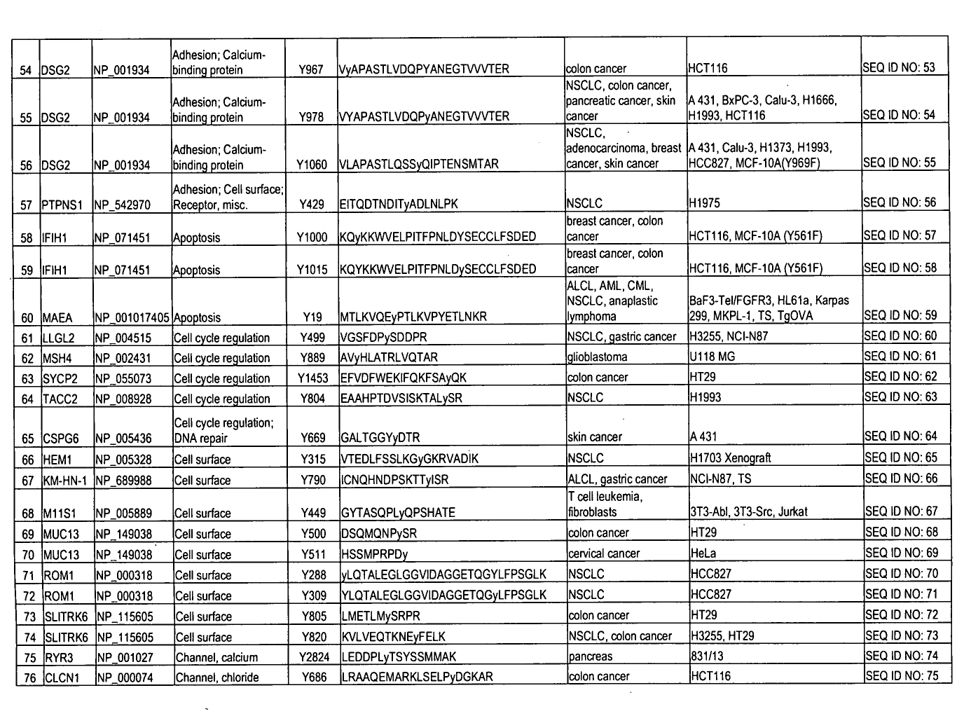

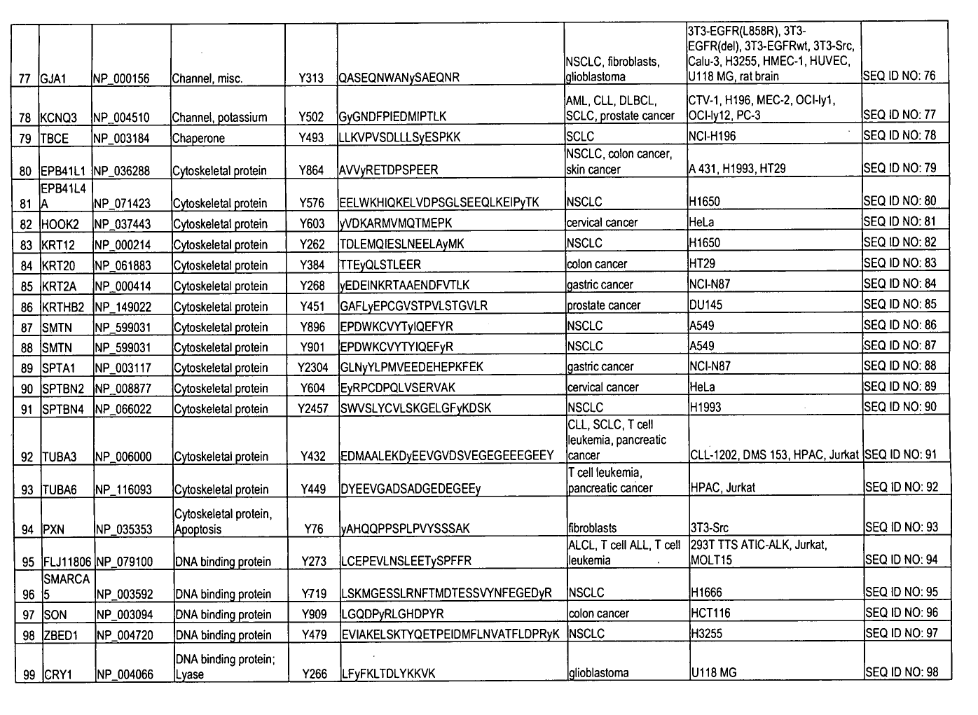

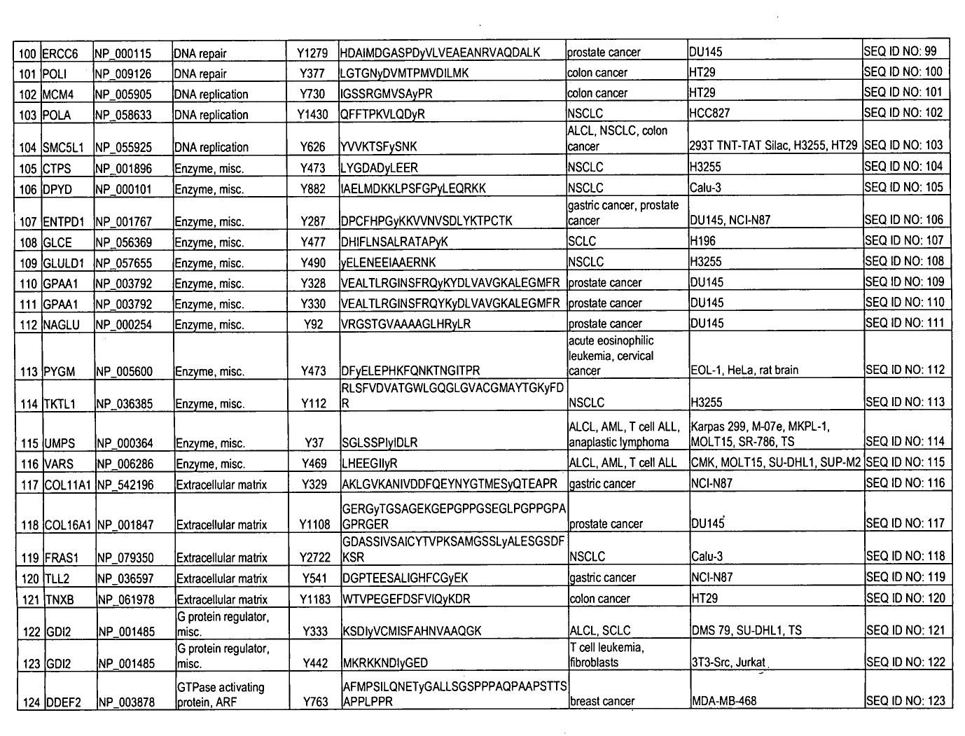

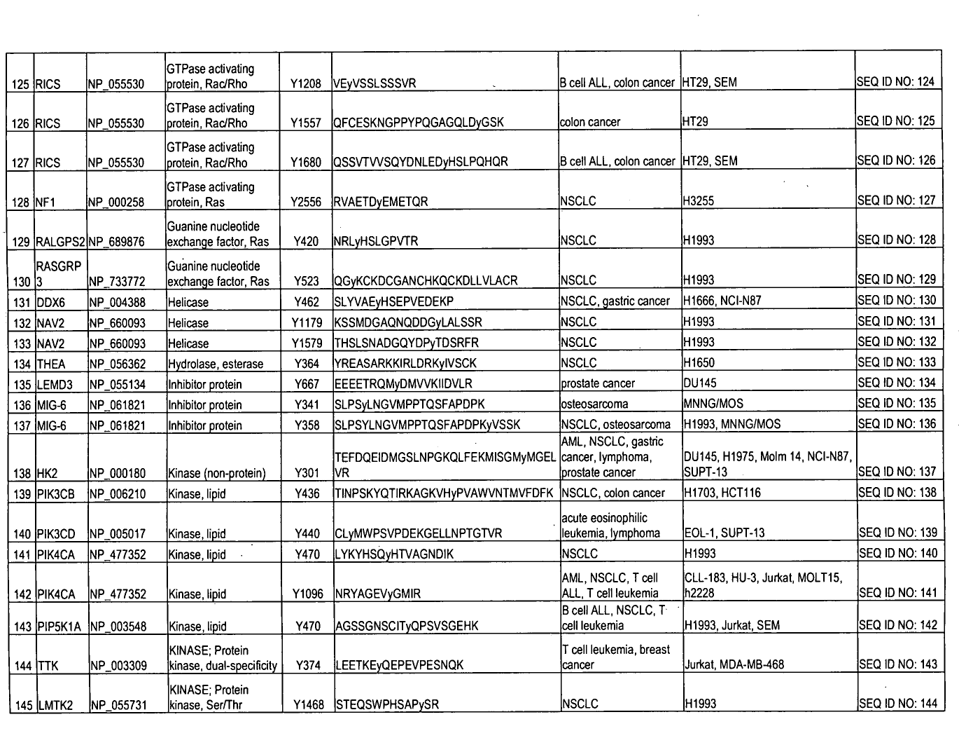

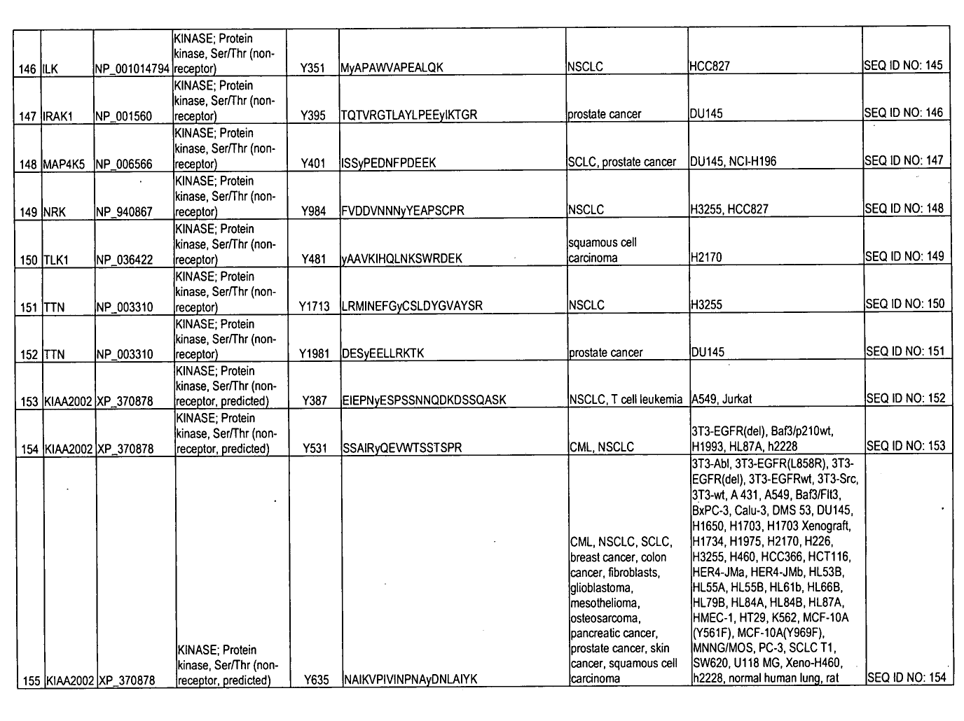

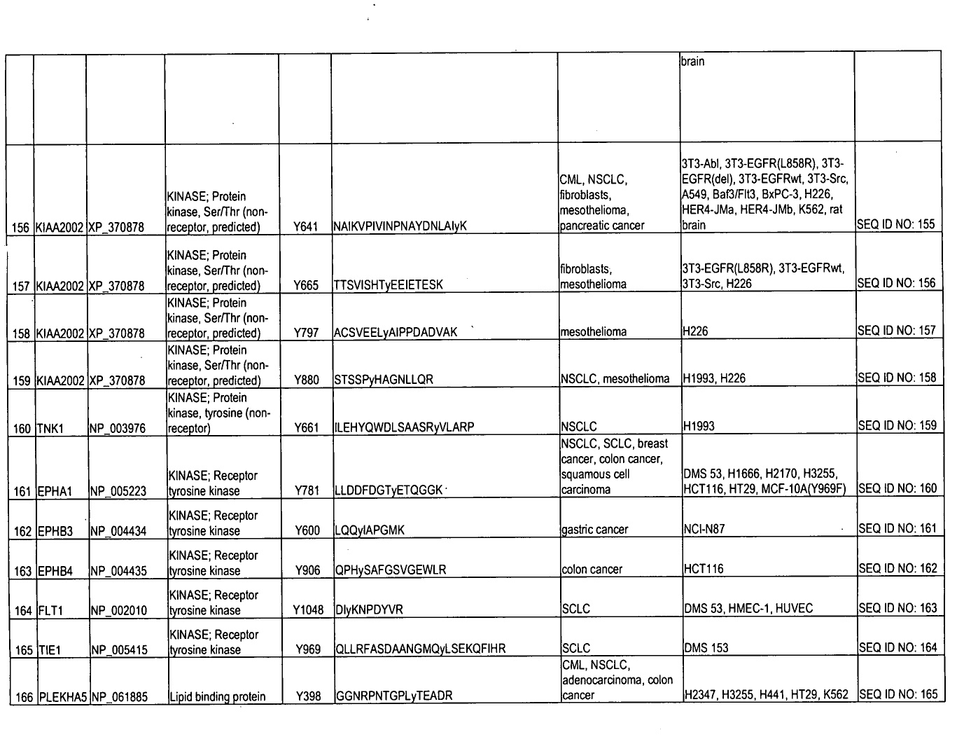

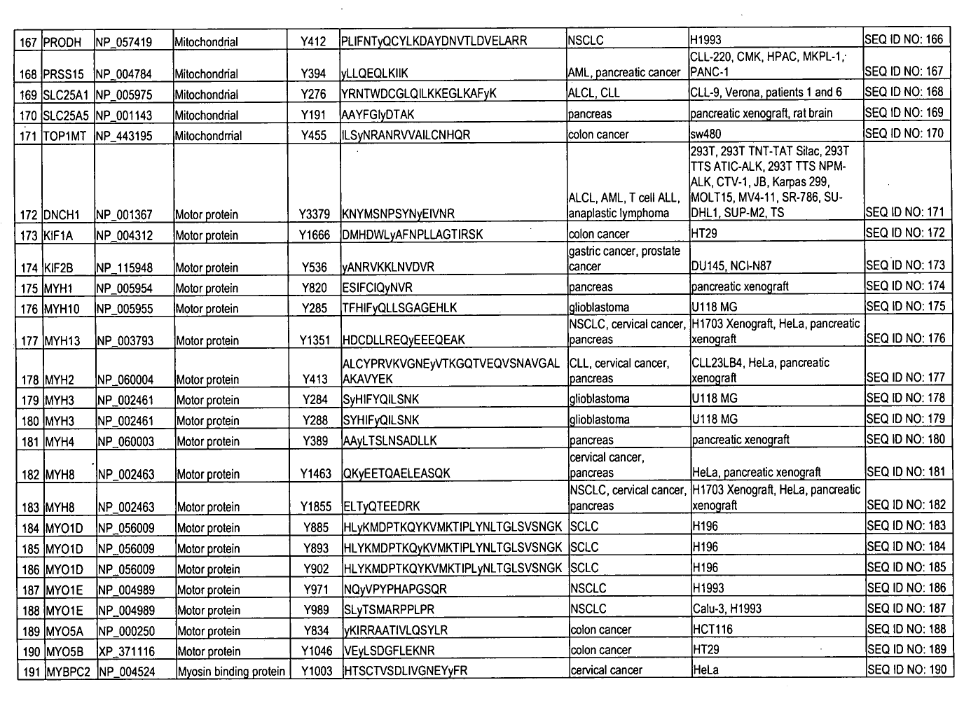

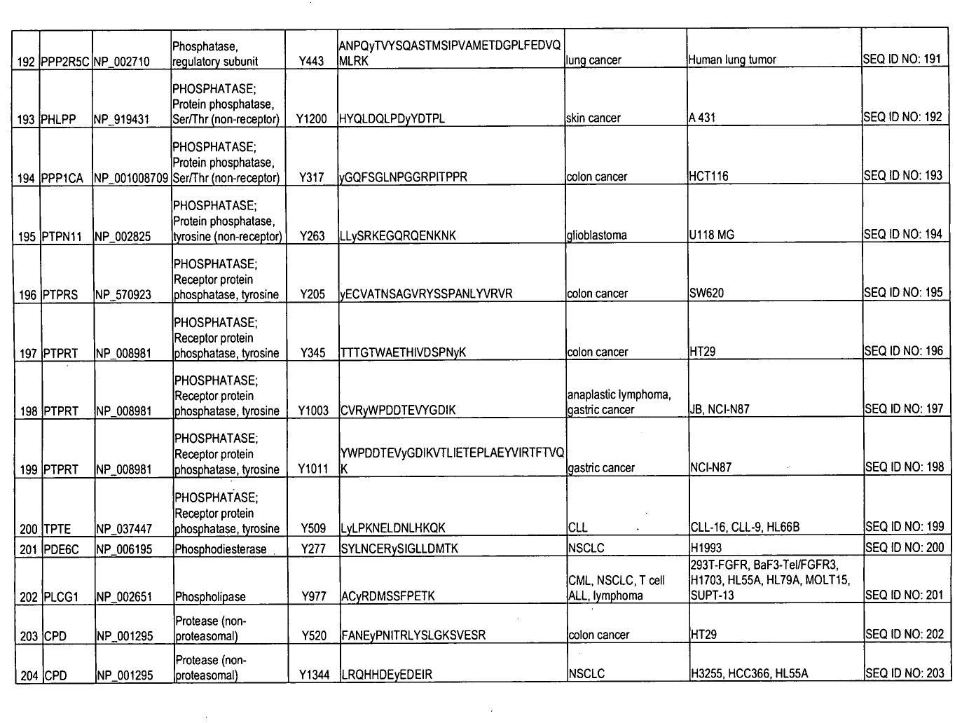

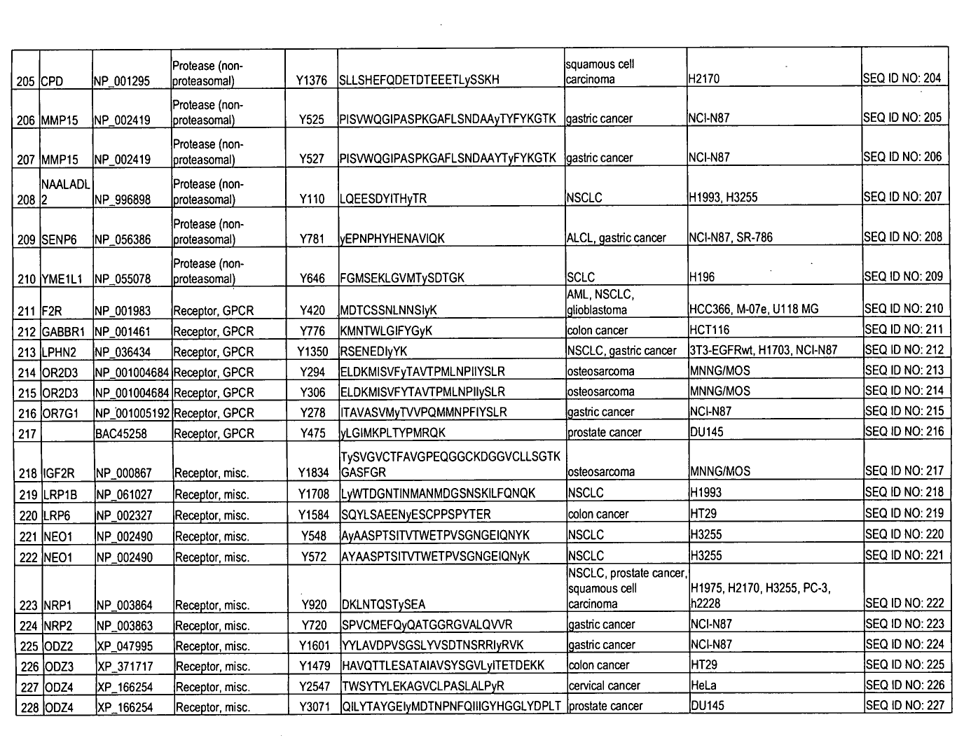

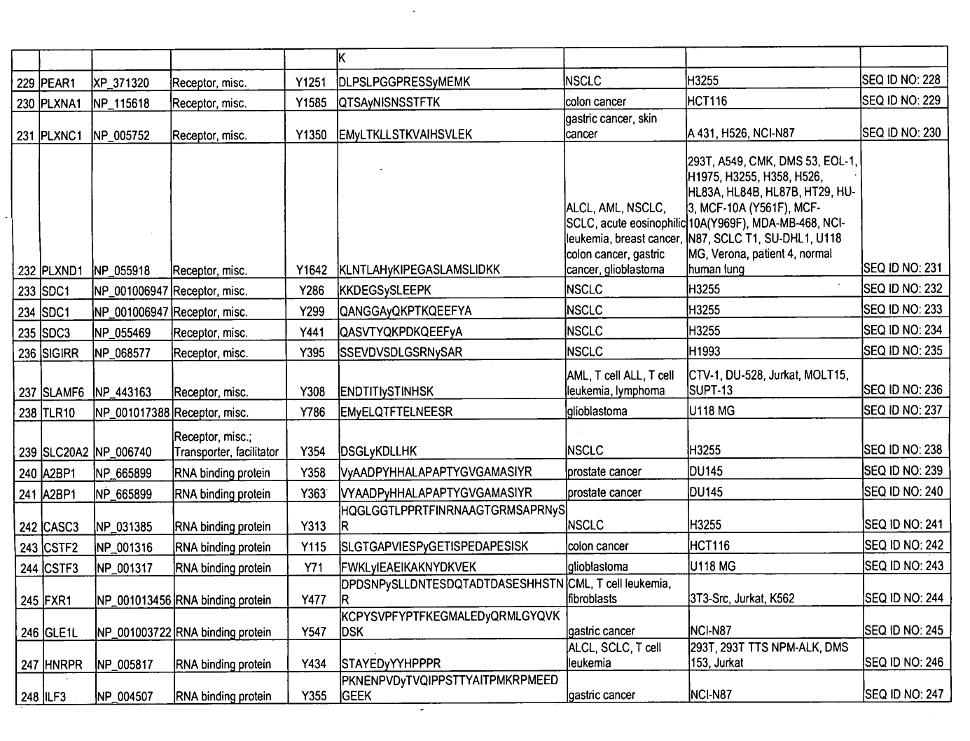

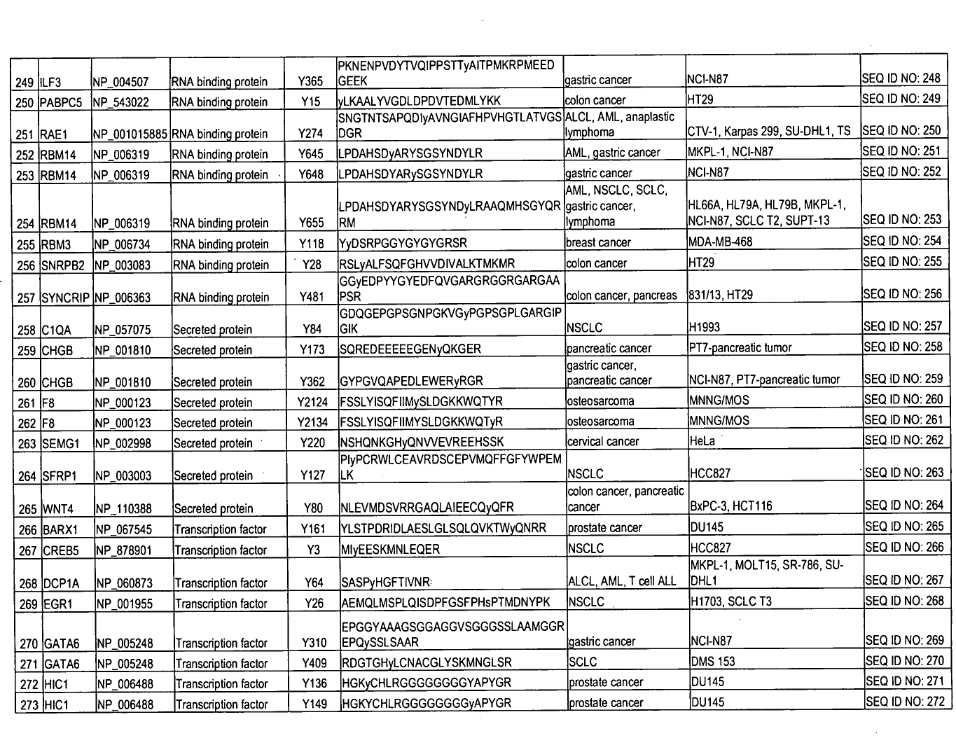

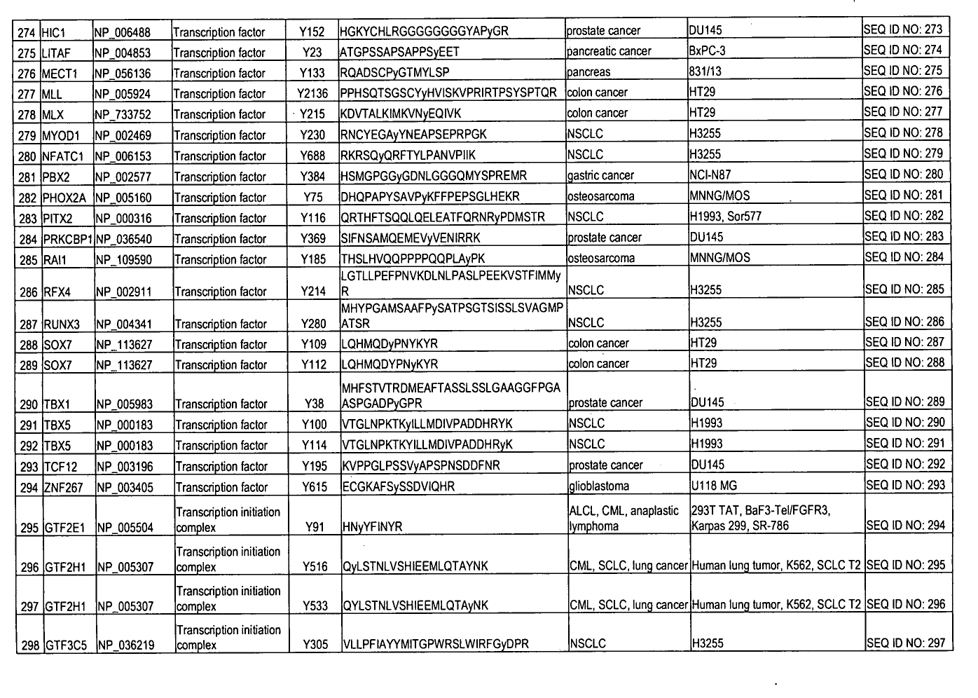

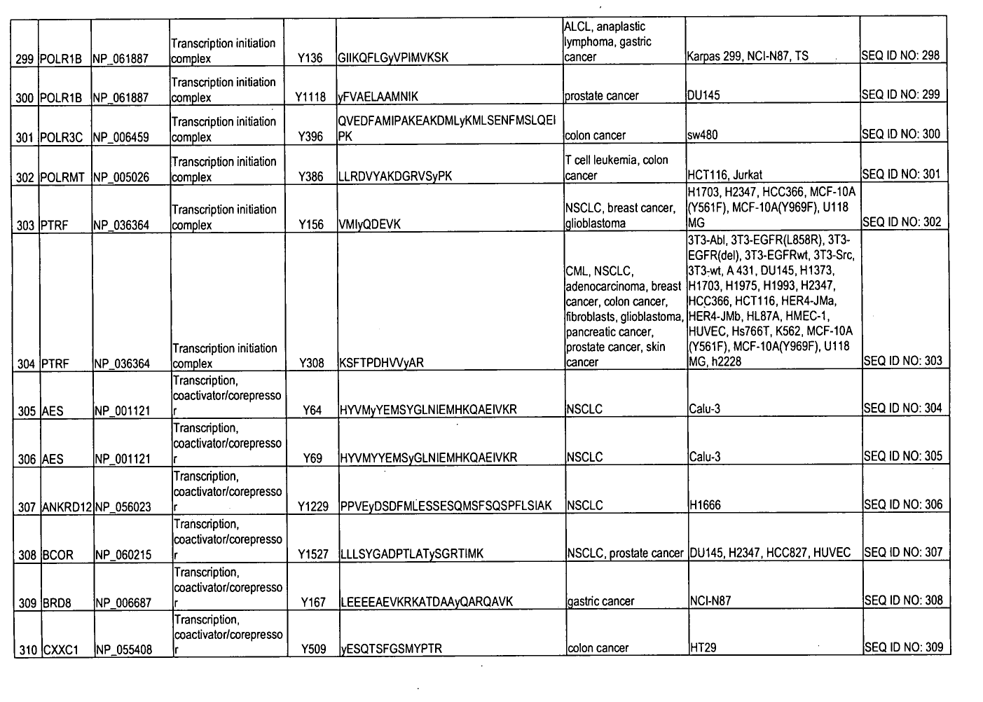

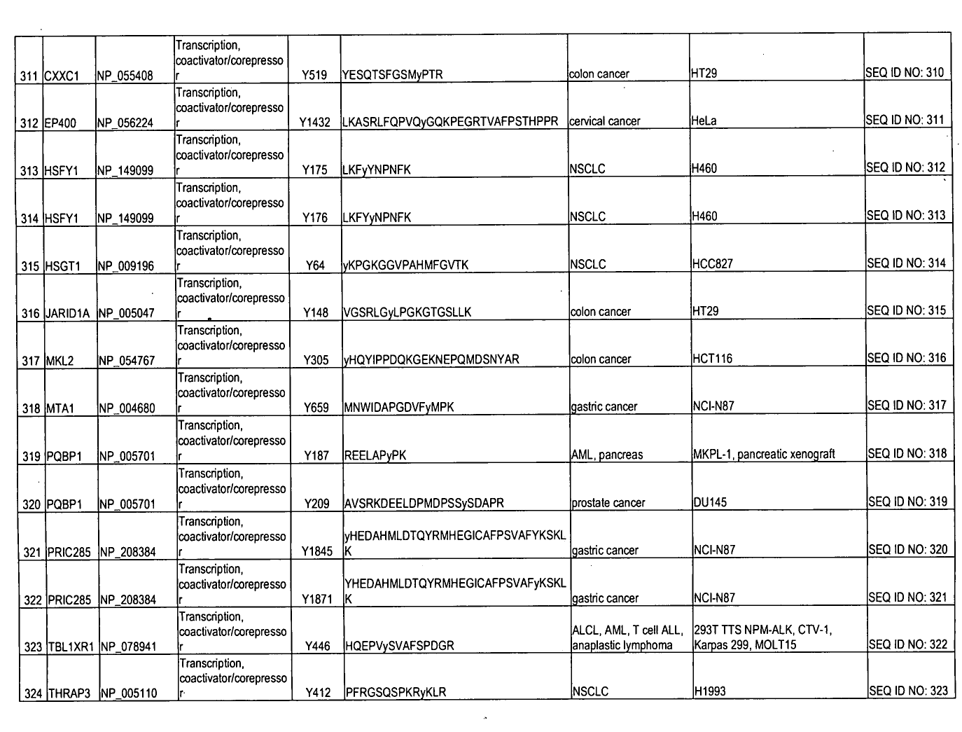

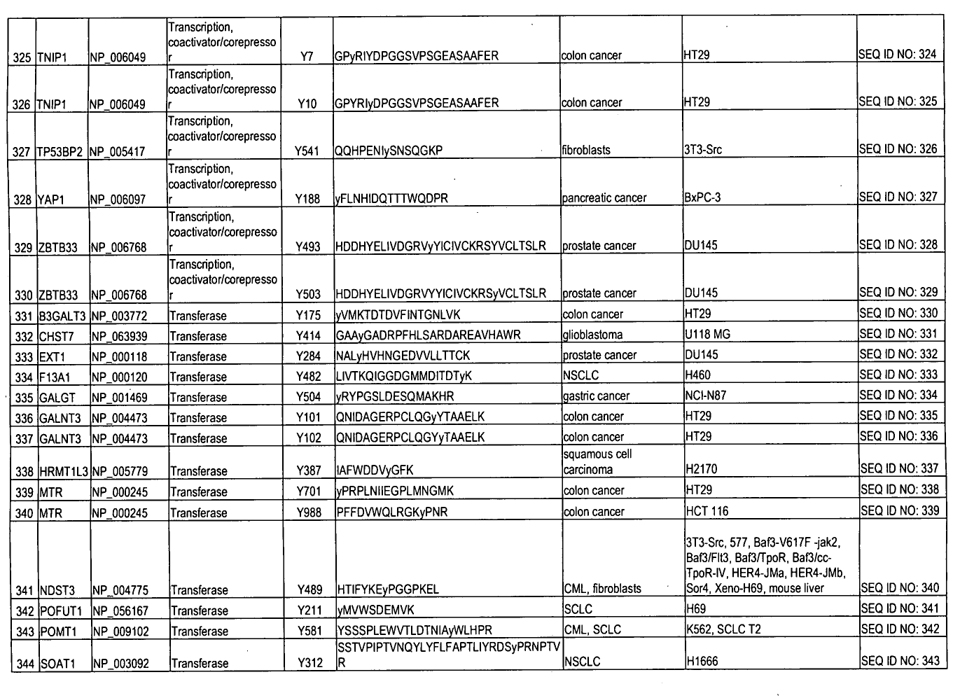

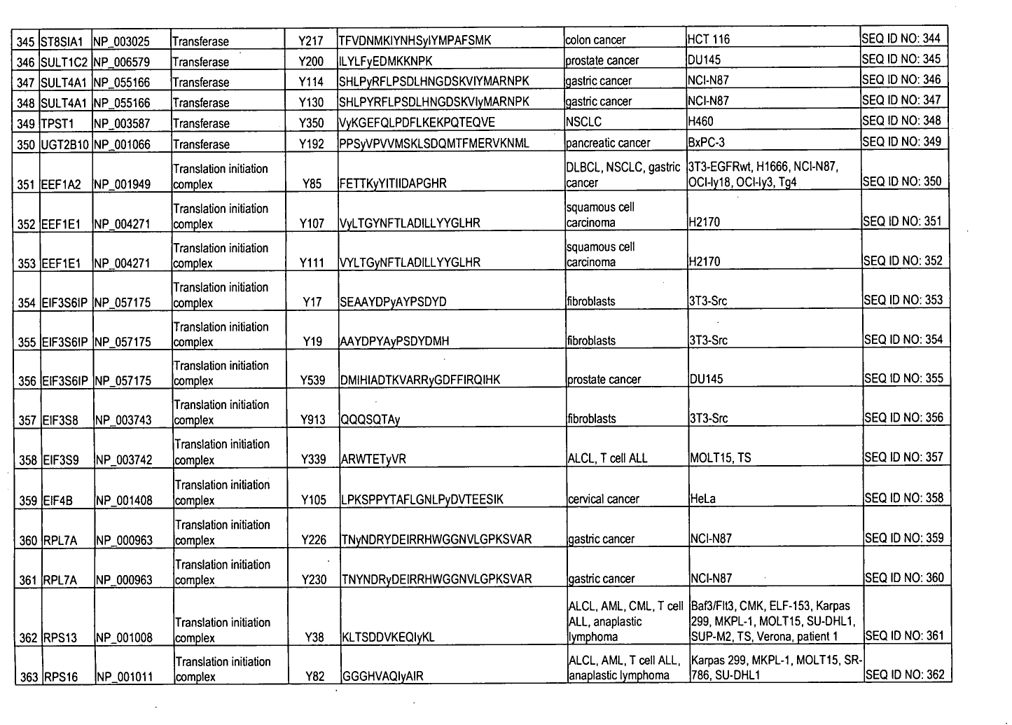

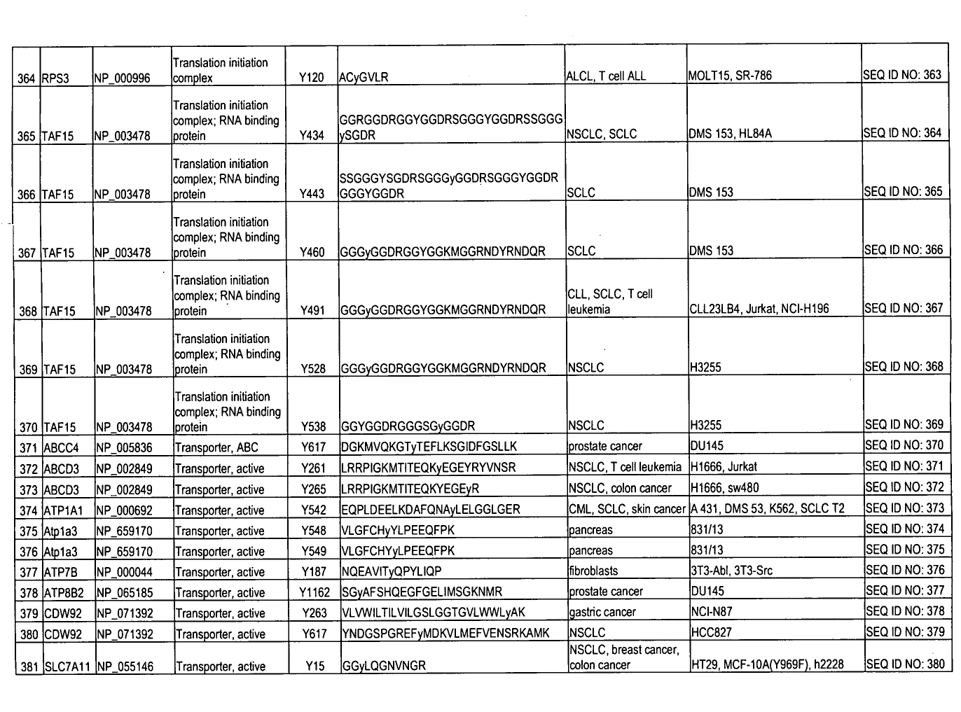

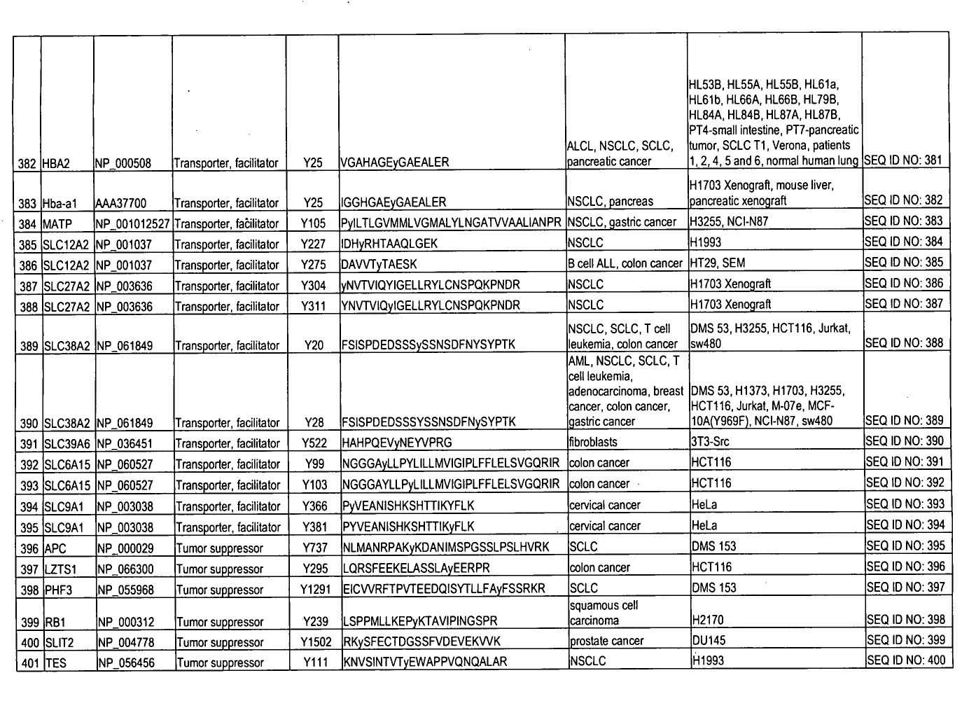

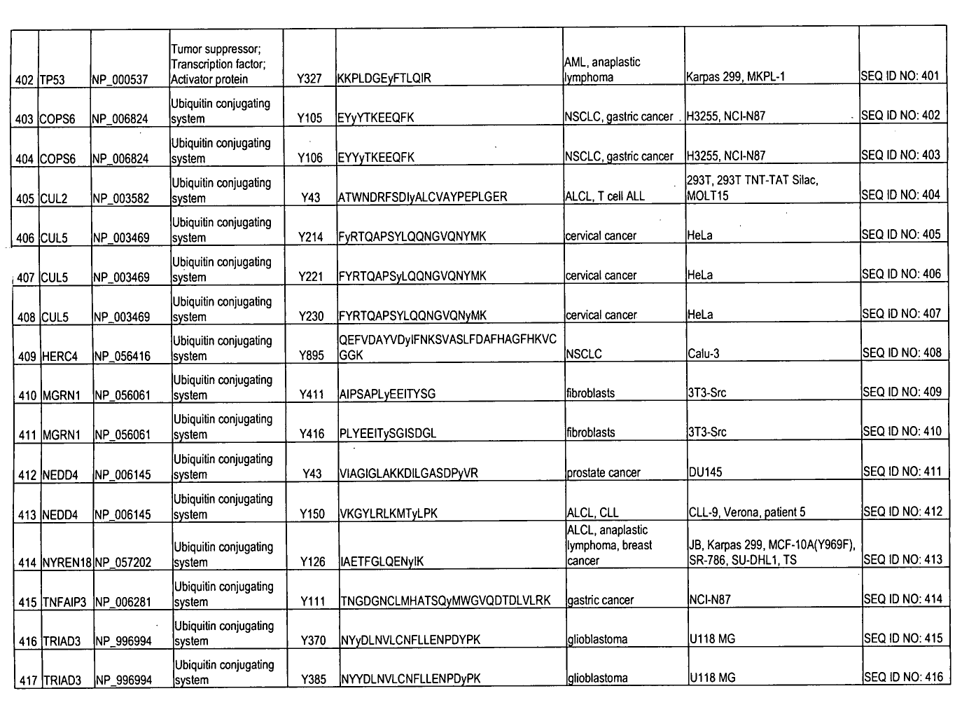

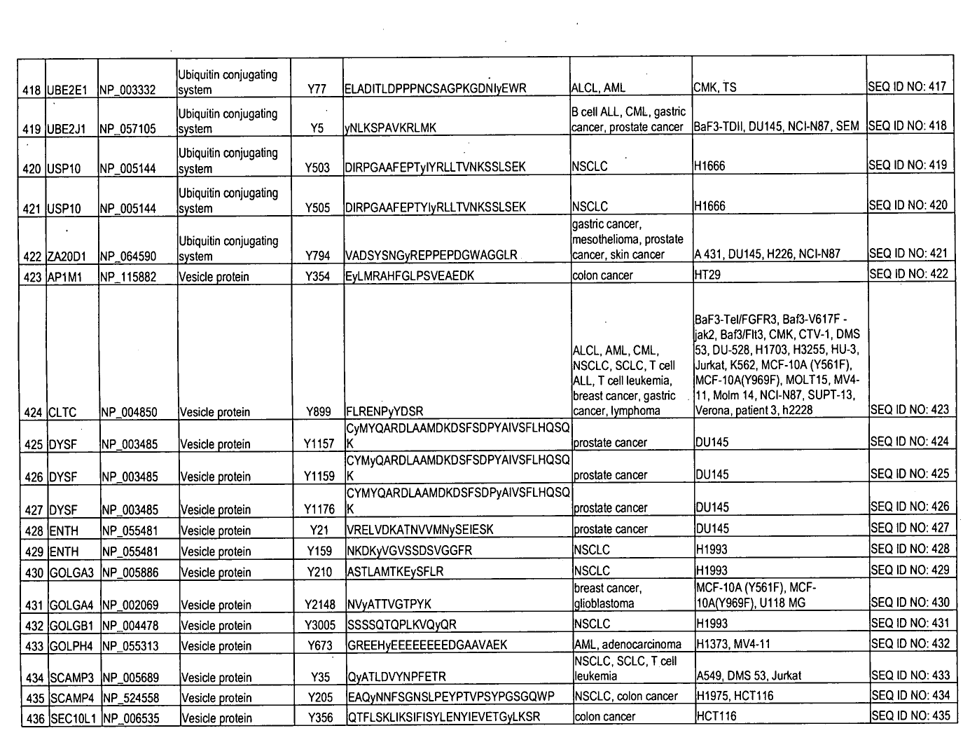

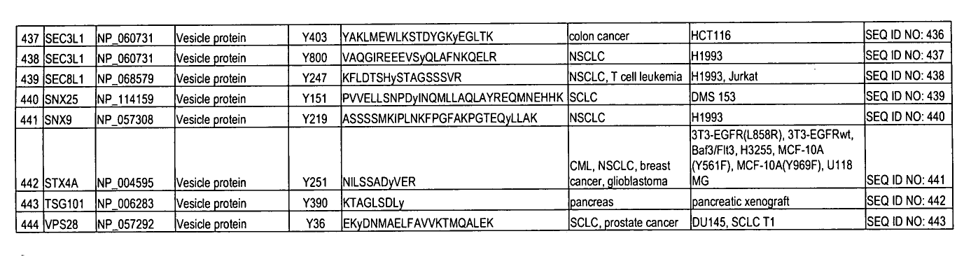

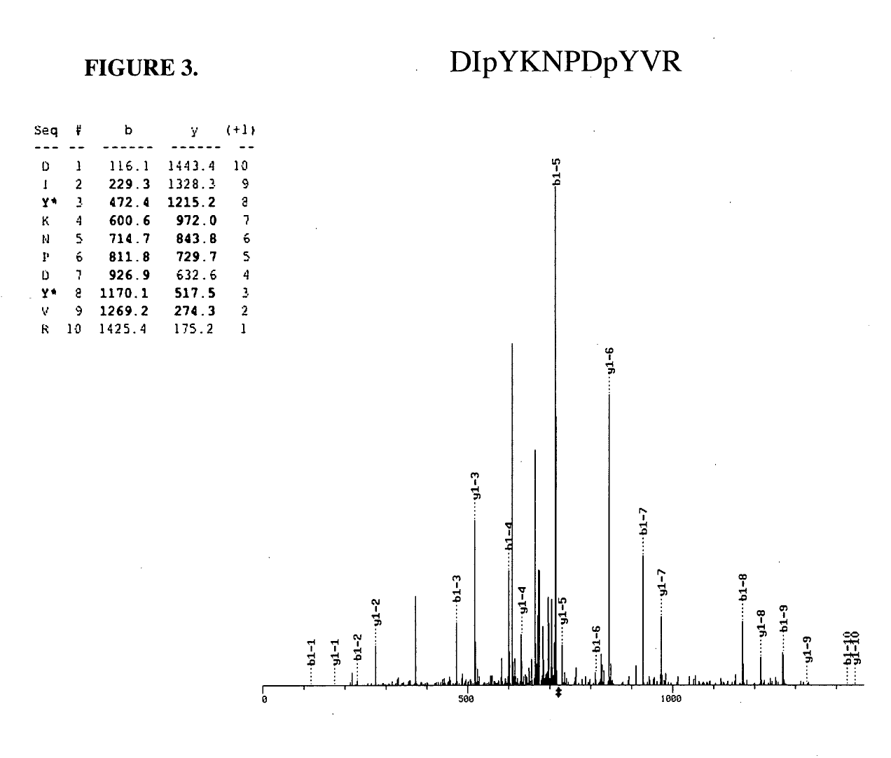

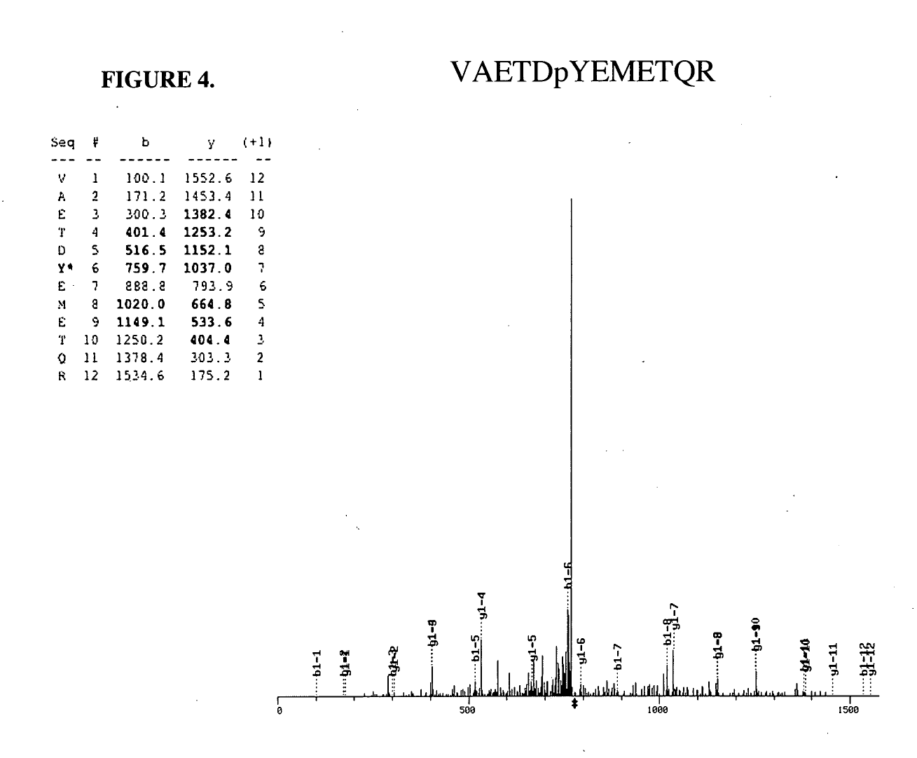

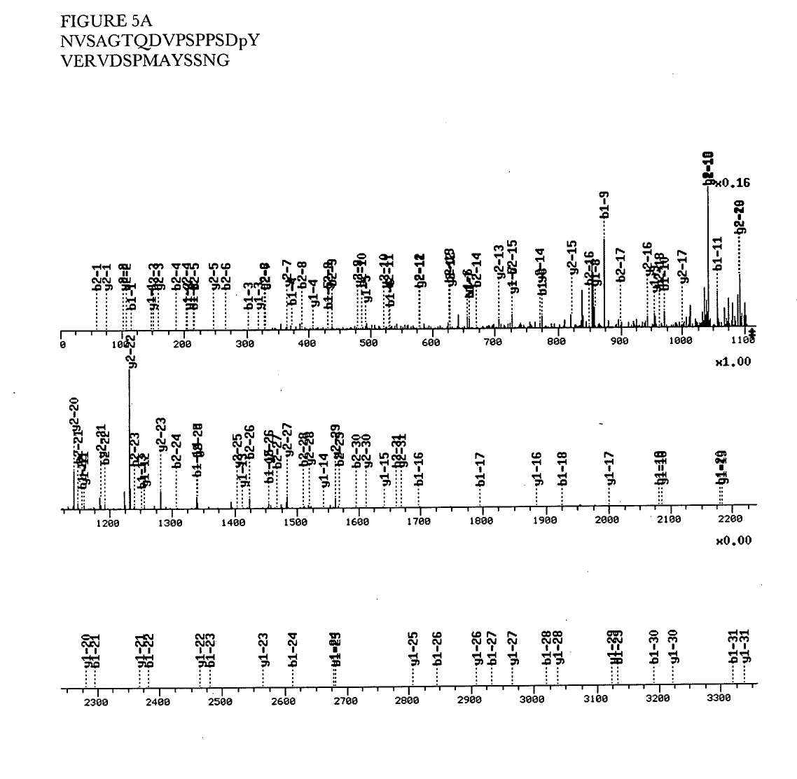

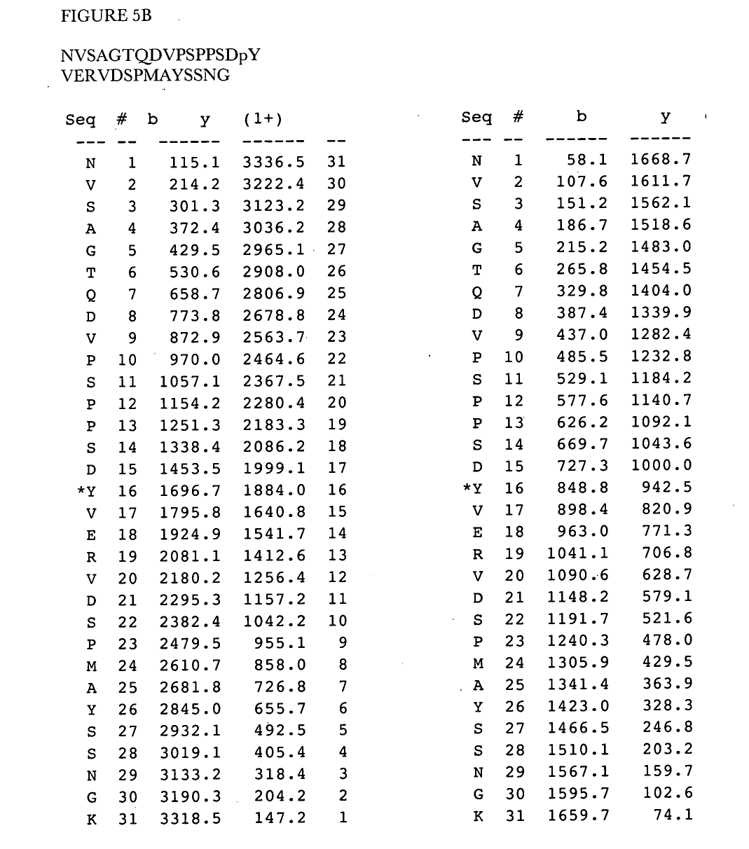

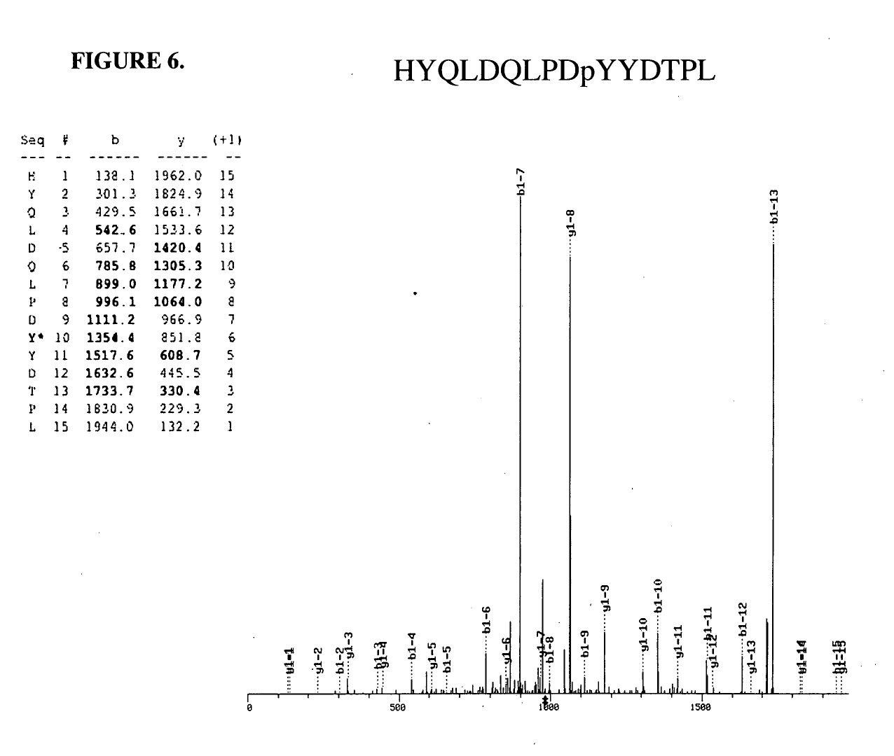

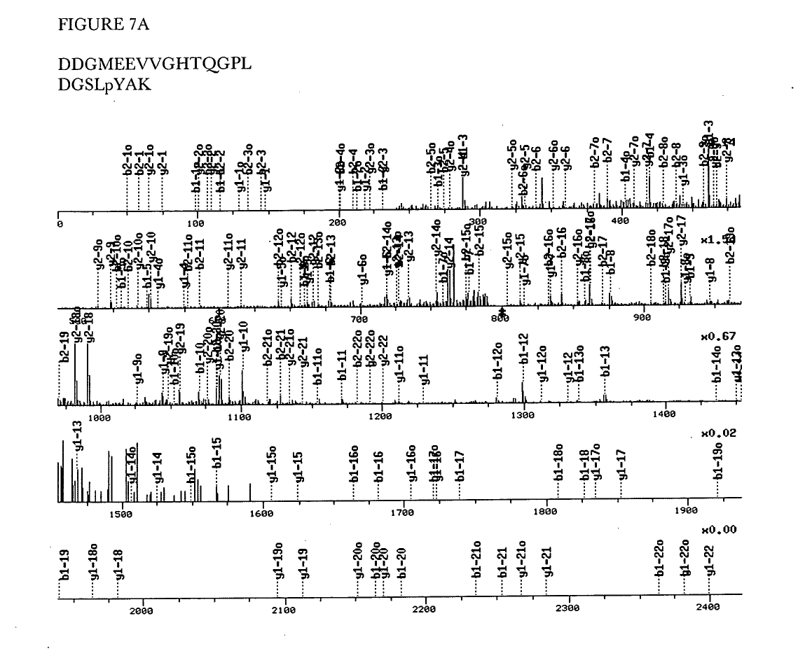

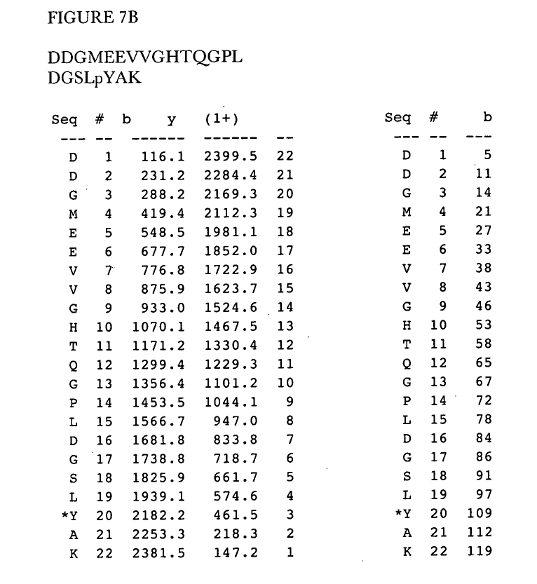

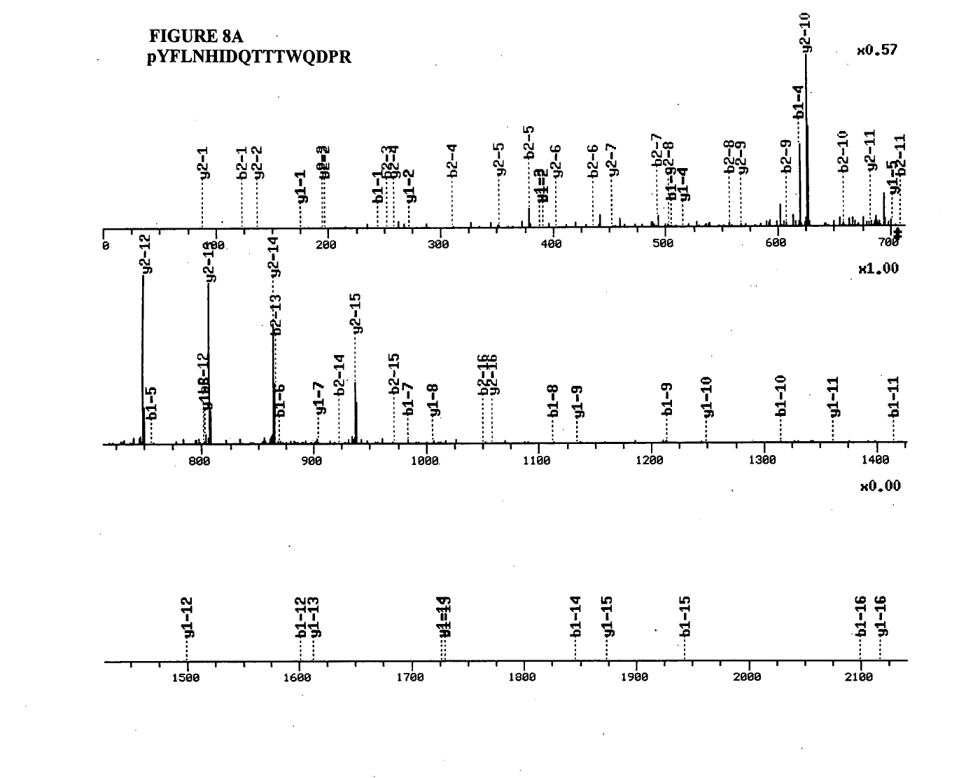

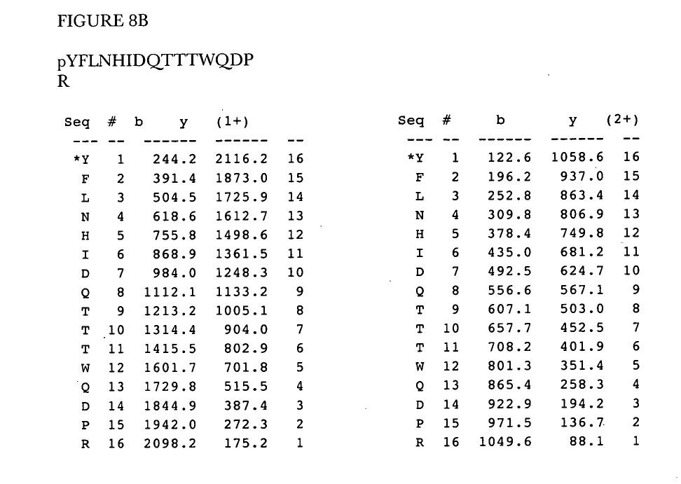

These phosphorylation sites correspond to numerous different parent proteins (the full sequences of which (human) are all publicly available in SwissProt database and their Accession numbers listed in Column B of Table 1/FIG. 2), each of which fall into discrete protein type groups, for example Protein Kinases (Serine/Threonine nonreceptor, Tyrosine receptor, Tyrosine nonreceptor, dual specificity and other), Adaptor/Scaffold proteins, transcription factors, phosphates, tumor suppressors, etc. (see Column C of Table 1), the phosphorylation of which is relevant to signal transduction activity underlying carcinomas (e.g., skin, lung, breast and colon cancer), as disclosed herein.

The discovery of the nearly 443 novel protein phosphorylation sites described herein enables the production, by standard methods, of new reagents, such as phosphorylation site-specific antibodies and AQUA peptides (heavy-isotope labeled peptides), capable of specifically detecting and/or quantifying these phosphorylated sites/proteins. Such reagents are highly useful, inter alia, for studying signal transduction events underlying the progression of carcinoma. Accordingly, the invention provides novel reagents—phospho-specific antibodies and AQUA peptides—for the specific detection and/or quantification of a Carcinoma-related signaling protein/polypeptide only when phosphorylated (or only when not phosphorylated) at a particular phosphorylation site disclosed herein. The invention also provides methods of detecting and/or quantifying one or more phosphorylated Carcinoma-related signaling proteins using the phosphorylation-site specific antibodies and AQUA peptides of the invention, and methods of obtaining a phosphorylation profile of such proteins (e.g. Kinases).

In part, the invention provides an isolated phosphorylation site-specific antibody that specifically binds a given Carcinoma-related signaling protein only when phosphorylated (or not phosphorylated, respectively) at a particular tyrosine enumerated in Column D of Table 1/FIG. 2 comprised within the phosphorylatable peptide site sequence enumerated in corresponding Column E. In further part, the invention provides a heavy-isotope labeled peptide (AQUA peptide) for the detection and quantification of a given Carcinoma-related signaling protein, the labeled peptide comprising a particular phosphorylatable peptide site/sequence enumerated in Column E of Table 1/FIG. 2 herein. For example, among the reagents provided by the invention is an isolated phosphorylation site-specific antibody that specifically binds the KIAA2002 kinase (serine/threonine) only when phosphorylated (or only when not phosphorylated) at tyrosine 635 (see Row 155 (and Columns D and E) of Table 1/FIG. 2). By way of further example, among the group of reagents provided by the invention is an AQUA peptide for the quantification of phosphorylated KIAA2002 kinase, the AQUA peptide comprising the phosphorylatable peptide sequence listed in Column E, Row 155 of Table 1/FIG. 2 (which encompasses the phosphorylatable tyrosine at position 635).

In one embodiment, the invention provides an isolated phosphorylation site-specific antibody that specifically binds a human Carcinoma-related signaling protein selected from Column A of Table 1 (Rows 2-444) only when phosphorylated at the tyrosine residue listed in corresponding Column D of Table 1, comprised within the phosphorylatable peptide sequence listed in corresponding Column E of Table 1 (SEQ ID NOs: 1, 3-8, 10-20, 22-24, 26-63, 65-67, 69-92, 94-154, 156-225, 227-243, 245-302, 304-325, 327-332, 334-340, 342-360, 362-365, 368-408, 411-432, and 434-443), wherein said antibody does not bind said signaling protein when not phosphorylated at said tyrosine. In another embodiment, the invention provides an isolated phosphorylation site-specific antibody that specifically binds a Carcinoma-related signaling protein selected from Column A of Table 1 only when not phosphorylated at the tyrosine residue listed in corresponding Column D of Table 1, comprised within the peptide sequence listed in corresponding Column E of Table 1 (SEQ ID NOs: 1, 3-8, 10-20, 22-24, 26-63, 65-67, 69-92, 94-154, 156-225, 227-243, 245-302, 304-325, 327-332, 334-340, 342-360, 362-365, 368-408, 411-432, and 434-443), wherein said antibody does not bind said signaling protein when phosphorylated at said tyrosine. Such reagents enable the specific detection of phosphorylation (or non-phosphorylation) of a novel phosphorylatable site disclosed herein. The invention further provides immortalized cell lines producing such antibodies. In one preferred embodiment, the immortalized cell line is a rabbit or mouse hybridoma.

In another embodiment, the invention provides a heavy-isotope labeled peptide (AQUA peptide) for the quantification of a Carcinoma-related signaling protein selected from Column A of Table 1, said labeled peptide comprising the phosphorylatable peptide sequence listed in corresponding Column E of Table 1 (SEQ ID NOs: 1, 3-8, 10-20, 22-24, 26-63, 65-67, 69-92, 94-154, 156-225, 227-243, 245-302, 304-325, 327-332, 334-340, 342-360, 362-365, 368-408, 411-432, and 434-443), which sequence comprises the phosphorylatable tyrosine listed in corresponding Column D of Table 1. In certain preferred embodiments, the phosphorylatable tyrosine within the labeled peptide is phosphorylated, while in other preferred embodiments, the phosphorylatable residue within the labeled peptide is not phosphorylated.

Reagents (antibodies and AQUA peptides) provided by the invention may conveniently be grouped by the type of Carcinoma-related signaling protein in which a given phosphorylation site (for which reagents are provided) occurs. The protein types for each respective protein (in which a phosphorylation site has been discovered) are provided in Column C of Table 1/FIG. 2, and include: Actin binding proteins, Adaptor/Scaffold proteins, Adhesion proteins, Apoptosis proteins, Cell Cycle Regulation proteins, Cell surface proteins, Channel proteins, Chaperone proteins, Cytoskeleton proteins, DNA binding proteins, DNA repair proteins, DNA replication proteins, Enzymes, Extracellular Matrix proteins, G protein regulatory proteins, GTPase activating proteins, Guanine nucleotide exchange factor proteins, Helicase proteins, Hydrolase proteins, Inhibitor proteins, Kinases (Serine/Threonine, dual specificity, Tyrosine etc.), Lipid binding proteins, Mitochondrial proteins, Motor proteins, Myosin biding proteins, Phosphatase proteins, Oxidoreductase proteins, Phospholipases, Proteases, Receptor proteins, RNA binding proteins, Secreted proteins, Transcription factor proteins, Transcription initiator complex proteins, Transcription coactivator/corepressor proteins, Transferase proteins, Translation initiation complex proteins, Transporter proteins, Tumor suppressor proteins, Ubiquitin conjugating proteins, and Vesicle proteins. Each of these distinct protein groups is considered a preferred subset of Carcinoma-related signal transduction protein phosphorylation sites disclosed herein, and reagents for their detection/quantification may be considered a preferred subset of reagents provided by the invention.

Particularly preferred subsets of the phosphorylation sites (and their corresponding proteins) disclosed herein are those occurring on the following protein types/groups listed in Column C of Table 1/FIG. 2: 1) Protein kinases (including Serine/Threonine dual specificity, and Tyrosine kinases), 2) Adaptor/Scaffold proteins, 3) Transcription factors, 4) Phospoatases, 5) Tumor supressors, 6) Ubiquitin conjugating system proteins, 7) Translation initiation complex proteins, 8) RNA binding proteins, 9) Apoptosis proteins, 10) Adhesion proteins, 11) G protein regulators/GTPase activating protein/Guanine nucleotide exchange factor proteins, and 12) DNA binding/replication/repair proteins. Accordingly, among preferred subsets of reagents provided by the invention are isolated antibodies and AQUA peptides useful for the detection and/or quantification of the foregoing preferred protein/phosphorylation site subsets.

In one subset of preferred embodiments there is provided:

(i) An isolated phosphorylation site-specific antibody that specifically binds a Protein kinase selected from Column A, Rows 138-165, of Table 1 only when phosphorylated at the tyrosine listed in corresponding Column D, Rows 138-165, of Table 1, comprised within the phosphorylatable peptide sequence listed in corresponding Column E, Rows 138-165, of Table 1 (SEQ ID NOs: 137-154, and 156-164), wherein said antibody does not bind said protein when not phosphorylated at said tyrosine.
(ii) An equivalent antibody to (i) above that only binds the Protein kinase when not phosphorylated at the disclosed site (and does not bind the protein when it is phosphorylated at the site).
(iii) A heavy-isotope labeled peptide (AQUA peptide) for the quantification of a Carcinoma-related signaling protein that is a Protein kinase selected from Column A, Rows 138-165, said labeled peptide comprising the phosphorylatable peptide sequence listed in corresponding Column E, Rows 138-165, of Table 1 (SEQ ID NOs: 137-154, and 156-164), which sequence comprises the phosphorylatable tyrosine listed in corresponding Column D, Rows 138-165, of Table 1.

Among this preferred subset of reagents, antibodies and AQUA peptides for the detection/quantification of the following Protein kinase phosphorylation sites are particularly preferred: PIK3CB (Y436), ILK (Y351), IRAK1 (Y395), KIAA2002 (Y635), and FLT1 (Y1048), (see SEQ ID NOs: 138, 145, 146, 154, and 163).

In one subset of preferred embodiments, there is provided:

(i) An isolated phosphorylation site-specific antibody that specifically binds an Adaptor/Scaffold protein selected from Column A, Rows 5-26, of Table 1 only when phosphorylated at the tyrosine listed in corresponding Column D, Rows 5-26, of Table 1, comprised within the phosphorylatable peptide sequence listed in corresponding Column E, Rows 5-26, of Table 1 (SEQ ID NOs: 4-8, 10-20, and 22-24), wherein said antibody does not bind said protein when not phosphorylated at said tyrosine.
(ii) An equivalent antibody to (i) above that only binds the Adaptor/Scaffold protein when not phosphorylated at the disclosed site (and does not bind the protein when it is phosphorylated at the site).
(iii) A heavy-isotope labeled peptide (AQUA peptide) for the quantification of a Carcinoma-related signaling protein that is an Adaptor/Scaffold protein selected from Column A, Rows 5-26, said labeled peptide comprising the phosphorylatable peptide sequence listed in corresponding Column E, Rows 5-26, of Table 1 (SEQ ID NOs: 4-8, 10-20, and 22-24), which sequence comprises the phosphorylatable tyrosine listed in corresponding Column D, Rows 5-26, of Table 1.

Among this preferred subset of reagents, antibodies and AQUA peptides for the detection/quantification of the following Adaptor/Scaffold protein phosphorylation site is particularly preferred: TNS1 (Y366), (see SEQ ID NO: 19).

In another subset of preferred embodiments there is provided:

(i) An isolated phosphorylation site-specific antibody that specifically binds a Transcription factor protein selected from Column A, Rows 266-330, of Table 1 only when phosphorylated at the tyrosine listed in corresponding Column D, Rows 266-330, of Table 1, comprised within the phosphorylatable peptide sequence listed in corresponding Column E, Rows 266-330, of Table 1 (SEQ ID NOs: 265-302, 304-325, and 327-329), wherein said antibody does not bind said protein when not phosphorylated at said tyrosine.
(ii) An equivalent antibody to (i) above that only binds the Transcription factor protein when not phosphorylated at the disclosed site (and does not bind the protein when it is phosphorylated at the site).
(iii) A heavy-isotope labeled peptide (AQUA peptide) for the quantification of a Carcinoma-related signaling protein that is a Transcription factor protein selected from Column A, Rows 266-330, said labeled peptide comprising the phosphorylatable peptide sequence listed in corresponding Column E, Rows 266-330, of Table 1 (SEQ ID NOs: 265-302, 304-325, and 327-329), which sequence comprises the phosphorylatable tyrosine listed in corresponding Column D, Rows 266-330, of Table 1.

Among this preferred subset of reagents, antibodies and AQUA peptides for the detection/quantification of the following Transcription factor protein phosphorylation sites are particularly preferred: HIC1 (Y136), MLL (Y2136), TBX1 (Y38), TBX5 (Y114), and YAP1 (Y188) (see SEQ ID NOs: 271, 276, 289, 291, and 327).

In still another subset of preferred embodiments, there is provided:

(i) An isolated phosphorylation site-specific antibody that specifically binds a Phosphatases selected from Column A, Rows 192-200, of Table 1 only when phosphorylated at the tyrosine listed in corresponding Column D, Rows 192-200, of Table 1, comprised within the phosphorylatable peptide sequence listed in corresponding Column E, Rows 192-200, of Table 1 (SEQ ID NOs: 191-199), wherein said antibody does not bind said protein when not phosphorylated at said tyrosine.
(ii) An equivalent antibody to (i) above that only binds the Phosphatase proteins when not phosphorylated at the disclosed site (and does not bind the protein when it is phosphorylated at the site).
(iii) A heavy-isotope labeled peptide (AQUA peptide) for the quantification of a Carcinoma-related signaling protein that is a Phosphatase selected from Column A, Rows 192-200, said labeled peptide comprising the phosphorylatable peptide sequence listed in corresponding Column E, Rows 192-200, of Table 1 (SEQ ID NOs: 191-199), which sequence comprises the phosphorylatable tyrosine listed in corresponding Column D, Rows 192-200, of Table 1.

Among this preferred subset of reagents, antibodies and AQUA peptides for the detection/quantification of the following Phosphatase phosphorylation sites are particularly preferred: PHLPP (Y1200), PTPN11 (Y263) and PTPRT (Y1003) (see SEQ ID NOs: 192, 194 and 197).

In still another subset of preferred embodiments there is provided:

(i) An isolated phosphorylation site-specific antibody that specifically binds a Tumor suppressor protein selected from Column A, Rows 396-402, of Table 1 only when phosphorylated at the tyrosine listed in corresponding Column D, Rows 396-402, of Table 1, comprised within the phosphorylatable peptide sequence listed in corresponding Column E, Rows 396-402, of Table 1 (SEQ ID NOs: 395-401), wherein said antibody does not bind said protein when not phosphorylated at said tyrosine.
(ii) An equivalent antibody to (i) above that only binds the Tumor suppressor protein when not phosphorylated at the disclosed site (and does not bind the protein when it is phosphorylated at the site).
(iii) A heavy-isotope labeled peptide (AQUA peptide) for the quantification of a Carcinoma-related signaling protein that is a Tumor suppressor protein selected from Column A, Rows 396-402, said labeled peptide comprising the phosphorylatable peptide sequence listed in corresponding Column E, Rows 396-402, of Table 1 (SEQ ID NOs: 395-401), which sequence comprises the phosphorylatable tyrosine listed in corresponding Column D, Rows 396-402, of Table 1.

Among this preferred subset of reagents, antibodies and AQUA peptides for the detection/quantification of the following Tumor suppressor phosphorylation sites are particularly preferred: APC (Y737), RB1 (Y239), and TP53 (Y327) (see SEQ ID NOs: 395, 398 and 401).

In still another subset of preferred embodiments there is provided:

(i) An isolated phosphorylation site-specific antibody that specifically binds a Ubiquitin conjugating system protein selected from Column A, Rows 403-422, of Table 1 only when phosphorylated at the tyrosine listed in corresponding Column D, Rows 403-422, of Table 1, comprised within the phosphorylatable peptide sequence listed in corresponding Column E, Rows 403-422, of Table 1 (SEQ ID NOs: 402-408, and 411-421), wherein said antibody does not bind said protein when not phosphorylated at said tyrosine.
(ii) An equivalent antibody to (i) above that only binds the Ubiquitin conjugating system protein when not phosphorylated at the disclosed site (and does not bind the protein when it is phosphorylated at the site).
(iii) A heavy-isotope labeled peptide (AQUA peptide) for the quantification of a Carcinoma-related signaling protein that is a Ubiquitin conjugating system protein selected from Column A, Rows 403-422, said labeled peptide comprising the phosphorylatable peptide sequence listed in corresponding Column E, Rows 403-422, of Table 1 (SEQ ID NOs: 402-408, and 411-421), which sequence comprises the phosphorylatable tyrosine listed in corresponding Column D, Rows 403-422, of Table 1.

Among this preferred subset of reagents, antibodies and AQUA peptides for the detection/quantification of the following Ubiquitin conjugating system protein phosphorylation sites are particularly preferred: CUL2 (Y43), CUL5 (Y214), and NEDD4 (Y43) (see SEQ ID NOs: 404, 405, and 411).

In still another subset of preferred embodiments there is provided:

(i) An isolated phosphorylation site-specific antibody that specifically binds a Translation initiation complex protein selected from Column A, Rows 351-370, of Table 1 only when phosphorylated at the tyrosine listed in corresponding Column D, Rows 351-370, of Table 1, comprised within the phosphorylatable peptide sequence listed in corresponding Column E, Rows 351-370 of Table 1 (SEQ ID NOs: 350-360, 362-365, and 368-369), wherein said antibody does not bind said protein when not phosphorylated at said tyrosine.
(ii) An equivalent antibody to (i) above that only binds the Translation initiation complex protein when not phosphorylated at the disclosed site (and does not bind the protein when it is phosphorylated at the site).
(iii) A heavy-isotope labeled peptide (AQUA peptide) for the quantification of a Carcinoma-related signaling protein that is a Translation initiation complex protein selected from Column A, Rows 351-370, said labeled peptide comprising the phosphorylatable peptide sequence listed in corresponding Column E, Rows 351-370, of Table 1 (SEQ ID NOs: 350-360, 362-365, and 368-369), which sequence comprises the phosphorylatable tyrosine listed in corresponding Column D, Rows 351-370, of Table 1.

Among this preferred subset of reagents, antibodies and AQUA peptides for the detection/quantification of the following Translation initiation complex protein phosphorylation site is particularly preferred: EIF4B (Y105) (see SEQ ID NO: 358).

In still another subset of preferred embodiments, there is provided:

(i) An isolated phosphorylation site-specific antibody that specifically binds an RNA binding protein selected from Column A, Rows 240-257, of Table 1 only when phosphorylated at the tyrosine listed in corresponding Column D, Rows 240-257, of Table 1, comprised within the phosphorylatable peptide sequence listed in corresponding Column E, Rows 240-257, of Table 1 (SEQ ID NOs: 239-243, and 245-256), wherein said antibody does not bind said protein when not phosphorylated at said tyrosine.
(ii) An equivalent antibody to (i) above that only binds the RNA binding protein when not phosphorylated at the disclosed site (and does not bind the protein when it is phosphorylated at the site).
(iii) A heavy-isotope labeled peptide (AQUA peptide) for the quantification of a Carcinoma-related signaling protein that is an RNA binding protein selected from Column A, Rows 240-257, said labeled peptide comprising the phosphorylatable peptide sequence listed in corresponding Column E, Rows 240-257, of Table 1 (SEQ ID NOs: 239-243, and 245-256), which sequence comprises the phosphorylatable tyrosine listed in corresponding Column D, Rows 240-257, of Table 1.

Among this preferred subset of reagents, antibodies and AQUA peptides for the detection/quantification of the following RNA binding protein phosphorylation sites are particularly preferred: RAE1 (Y274) (see SEQ ID NO: 250).

In yet another subset of preferred embodiments, there is provided:

(i) An isolated phosphorylation site-specific antibody that specifically binds an Apoptosis protein selected from Column A, Rows 58-60, of Table 1 only when phosphorylated at the tyrosine listed in corresponding Column D, Rows 58-60, of Table 1, comprised within the phosphorylatable peptide sequence listed in corresponding Column E, Rows 58-60, of Table 1 (SEQ ID NOs: 57-59), wherein said antibody does not bind said protein when not phosphorylated at said tyrosine.
(ii) An equivalent antibody to (i) above that only binds the Apoptosis protein when not phosphorylated at the disclosed site (and does not bind the protein when it is phosphorylated at the site).
(iii) A heavy-isotope labeled peptide (AQUA peptide) for the quantification of a Carcinoma-related signaling protein that is an Apoptosis protein selected from Column A, Rows 58-60, said labeled peptide comprising the phosphorylatable peptide sequence listed in corresponding Column E, Rows 58-60, of Table 1 (SEQ ID NOs: 57-59), which sequence comprises the phosphorylatable tyrosine listed in corresponding Column D, Rows 58-60, of Table 1.

Among this preferred subset of reagents, antibodies and AQUA peptides for the detection/quantification of the following Apoptosis protein phosphorylation sites are particularly preferred: IFIH1 (Y1000) (see SEQ ID NO: 57).

In yet another subset of preferred embodiments, there is provided:

(i) An isolated phosphorylation site-specific antibody specifically binds an Adhesion protein selected from Column A, Rows 27-57, of Table 1 only when phosphorylated at the tyrosine listed in corresponding to Column D, Rows 27-57, of Table 1, comprised within the phosphorylatable peptide sequence listed in corresponding Column E, Rows 27-57, of Table 1 (SEQ ID NOs: 26-56), wherein said antibody does not bind said protein when not phosphorylated at said tyrosine.
(ii) An equivalent antibody to (i) above that only binds the Adhesion protein when not phosphorylated at the disclosed site (and does not bind the protein when it is phosphorylated at the site).
(iii) A heavy-isotope labeled peptide (AQUA peptide) for the quantification of a Carcinoma-related signaling protein that is an Adhesion protein selected from Column A, Rows 27-57, said labeled peptide comprising the phosphorylatable peptide sequence listed in corresponding Column E, Rows 27-57, of Table 1 (SEQ ID NOs: 26-56), which sequence comprises the phosphorylatable tyrosine listed in corresponding Column D, Rows 27-57, of Table 1.

Among this preferred subset of reagents, antibodies and AQUA peptides for the detection/quantification of the following Adhesion protein phosphorylation sites are particularly preferred: F11R (Y280), OCLN (Y315) (see SEQ ID NOs: 33 and 43).

In yet another subset of preferred embodiments, there is provided:

(i) An isolated phosphorylation site-specific antibody that specifically binds a G protein regulator proteins/GTPase activating proteins/Guanine nucleotide exchange factor proteins selected from Column A, Rows 122-130, of Table 1 only when phosphorylated at the tyrosine listed in corresponding Column D, Rows 122-130, of Table 1, comprised within the phosphorylatable peptide sequence listed in corresponding Column E, Rows 122-130, of Table 1 (SEQ ID NOs: 121-129), wherein said antibody does not bind said protein when not phosphorylated at said tyrosine.
(ii) An equivalent antibody to (i) above that only binds the G protein regulator proteins/GTPase activating proteins/Guanine nucleotide exchange factor proteins when not phosphorylated at the disclosed site (and does not bind the protein when it is phosphorylated at the site).
(iii) A heavy-isotope labeled peptide (AQUA peptide) for the quantification of a Carcinoma-related signaling protein that is a G protein regulator proteins/GTPase activating proteins/Guanine nucleotide exchange factor proteins selected from Column A, Rows 122-130, said labeled peptide comprising the phosphorylatable peptide sequence listed in corresponding Column E, Rows 122-130, of Table 1 (SEQ ID NOs: 121-129), which sequence comprises the phosphorylatable tyrosine listed in corresponding Column D, Rows 122-130, of Table 1.

Among this preferred subset of reagents, antibodies and AQUA peptides for the detection/quantification of the following G protein regulator proteins/GTPase activating proteins/Guanine nucleotide exchange factor proteins phosphorylation sites are particularly preferred: NF1 (Y2556), RASGRP3 (Y523) (see SEQ ID NOs: 127 and 129).

In still another subset of preferred embodiments, there is provided:

(i) An isolated phosphorylation site-specific antibody that specifically binds a DNA binding/replication/repair protein selected from Column A, Rows 95-104, of Table 1 only when phosphorylated at the tyrosine listed in corresponding Column D, Rows 95-104, of Table 1, comprised within the phosphorylatable peptide sequence listed in corresponding Column E, Rows 95-104, of Table 1 (SEQ ID NOs: 94-103), wherein said antibody does not bind said protein when not phosphorylated at said tyrosine.
(ii) An equivalent antibody to (i) above that only binds the DNA binding/replication/repair protein when not phosphorylated at the disclosed site (and does not bind the protein when it is phosphorylated at the site).
(iii) A heavy-isotope labeled peptide (AQUA peptide) for the quantification of a Carcinoma-related signaling protein that is a DNA binding/replication/repair protein selected from Column A, Rows 95-104, said labeled peptide comprising the phosphorylatable peptide sequence listed in corresponding Column E, Rows 95-104, of Table 1 (SEQ ID NOs: 94-103), which sequence comprises the phosphorylatable tyrosine listed in corresponding Column D, Rows 95-104, of Table 1.

Among this preferred subset of reagents, antibodies and AQUA peptides for the detection/quantification of the following DNA binding/replication/repair protein phosphorylation sites are particularly preferred: SMARCA5 (Y719) (see SEQ ID NO: 95).

In still another subset of preferred embodiments, there is provided:

(i) An isolated phosphorylation site-specific antibody that specifically binds the Receptor protein of Row 218, of Table 1 only when phosphorylated at the tyrosine listed in corresponding Column D, Row 218 of Table 1, comprised within the phosphorylatable peptide sequence listed in corresponding Column E, Row 218 of Table 1 (SEQ ID NO: 217), wherein said antibody does not bind said protein when not phosphorylated at said tyrosine.
(ii) An equivalent antibody to (i) above that only binds the Receptor protein when not phosphorylated at the disclosed site (and does not bind the protein when it is phosphorylated at the site).
(iii) A heavy-isotope labeled peptide (AQUA peptide) for the quantification of a Carcinoma-related signaling protein that is the Receptor protein of Column A, Row 218, said labeled peptide comprising the phosphorylatable peptide sequence listed in corresponding Column E, Row 218 of Table 1 (SEQ ID NO: 217), which sequence comprises the phosphorylatable tyrosine listed in corresponding Column D, Rows 217 of Table 1.

The invention also provides, in part, an immortalized cell line producing an antibody of the invention, for example, a cell line producing an antibody within any of the foregoing preferred subsets of antibodies. In one preferred embodiment, the immortalized cell line is a rabbit hybridoma or a mouse hybridoma.

In certain other preferred embodiments, a heavy-isotope labeled peptide (AQUA peptide) of the invention (for example, an AQUA peptide within any of the foregoing preferred subsets of AQUA peptides) comprises a disclosed site sequence wherein the phosphorylatable tyrosine is phosphorylated. In certain other preferred embodiments, a heavy-isotope labeled peptide of the invention comprises a disclosed site sequence wherein the phosphorylatable tyrosine is not phosphorylated.

The foregoing subsets of preferred reagents of the invention should not be construed as limiting the scope of the invention, which, as noted above, includes reagents for the detection and/or quantification of disclosed phosphorylation sites on any of the other protein type/group subsets (each a preferred subset) listed in Column C of Table 1/FIG. 2.

Also provided by the invention are methods for detecting or quantifying a Carcinoma-related signaling protein that is tyrosine phosphorylated, said method comprising the step of utilizing one or more of the above-described reagents of the invention to detect or quantify one or more Carcinoma-related signaling protein(s) selected from Column A of Table 1 only when phosphorylated at the tyrosine listed in corresponding Column D of Table 1. In certain preferred embodiments of the methods of the invention, the reagents comprise a subset of preferred reagents as described above.

Also provided by the invention is a method for obtaining a phosphorylation profile of protein kinases that are phosphorylated in Carcinoma signaling pathways, said method comprising the step of utilizing one or more isolated antibody that specifically binds a protein kinase selected from Column A, Rows 138-165, of Table 1 only when phosphorylated at the tyrosine listed in corresponding Column D, Rows 138-165, of Table 1, comprised within the phosphorylation site sequence listed in corresponding Column E, Rows 138-165, of Table 1 (SEQ ID NOs: 137-154, and 156-164), to detect the phosphorylation of one or more of said protein kinases, thereby obtaining a phosphorylation profile for said kinases.

The identification of the disclosed nearly 443 novel Carcinoma-related signaling protein phosphorylation sites, and the standard production and use of the reagents provided by the invention are described in further detail below and in the Examples that follow.

All cited references are hereby incorporated herein, in their entirety, by reference. The Examples are provided to further illustrate the invention, and do not in any way limit its scope, except as provided in the claims appended hereto.

TABLE 1

Newly Discovered Carcinoma-Related
Signaling Protein Phosphorylation Sites.

| | | | | | | |
|---|---|---|---|---|---|---|
| | A | B | C | D | E | H |
| | Protein | Accession | Protein | Phospho | Phosphorylation | |
| 1 | Name | No. | Type | Residue | Site Sequence | SEQ ID NO |

| | | | | | | |
|---|---|---|---|---|---|---|
| 2 | FSCN2 | NP_036550.1 | Actin binding protein | Y228 | yLAPVGPAGTLKAGRNTR | SEQ ID NO: 1 |
| 3 | TENC1 | | Actin binding protein | Y493 | GPLDGSPyAQVQR | SEQ ID NO: 2 |
| 4 | TENC1 | NP_056134.2 | Actin binding protein | Y780 | AGEEGHEGCSyTMCPEGR | SEQ ID NO: 3 |
| 5 | DLG5 | NP_004738.3 | Adaptor/scaffold | Y71 | LAFATHGTAFDKRPyHR | SEQ ID NO: 4 |
| 6 | DLG5 | NP_004738.3 | Adaptor/scaffold | Y1133 | LSLDLSHRTCSDySEMR | SEQ ID NO: 5 |
| 7 | IRS4 | NP_003595.1 | Adaptor/scaffold | Y743 | GyMMMFPR | SEQ ID NO: 6 |
| 8 | IRS4 | NP_003595.1 | Adaptor/scaffold | Y808 | SWSSyFSLPNPFR | SEQ ID NO: 7 |
| 9 | IRS4 | NP_003595.1 | Adaptor/scaffold | Y828 | SSPLGQNDNSEyVPMLPGK | SEQ ID NO: 8 |
| 10 | IRS4 | | Adaptor/scaffold | Y921 | EADSSSDyVNMDFTK | SEQ ID NO: 9 |
| 11 | KPNA5 | NP_002260.2 | Adaptor/scaffold | Y17 | MDAMASPGKDNYRMKSyK | SEQ ID NO: 10 |
| 12 | PARD3 | NP_062565.2 | Adaptor/scaffold | Y489 | DVTIGGSAPIyVK | SEQ ID NO: 11 |
| 13 | PARD3 | NP_062565.2 | Adaptor/scaffold | Y1310 | KEQQMKKQPPSEGPSNyDSYK | SEQ ID NO: 12 |
| 14 | RAPH1 | NP_998754.1 | Adaptor/scaffold | Y1226 | AGYGGSHISGyATLR | SEQ ID NO: 13 |
| 15 | SHANK2 | NP_036441.1 | Adaptor/scaffold | Y322 | VyGTIKPAFNQNSAAK | SEQ ID NO: 14 |
| 16 | SHANK2 | NP_036441.1 | Adaptor/scaffold | Y372 | ELDRYSLDSEDLySR | SEQ ID NO: 15 |
| 17 | SHANK2 | NP_036441.1 | Adaptor/scaffold | Y606 | AQGPESSPAVPSASSGTAGPGNyVHPLT | SEQ ID NO: 16 |
| | | | | | GR | |
| 18 | SORBS1 | NP_006425.2 | Adaptor/scaffold | Y555 | GERITLLRQVDENWyEGR | SEQ ID NO: 17 |
| 19 | TJP2 | NP_004808.2 | Adaptor/scaffold | Y426 | HQYSDyDYHSSSEK | SEQ ID NO: 18 |
| 20 | TNS1 | NP_072174.3 | Adaptor/scaffold | Y366 | DDGMEEVVGHTQGPLDGSLyAK | SEQ ID NO: 19 |
| 21 | TNS1 | NP_072174.3 | Adaptor/scaffold | Y1254 | HPAGVyQVSGLHNK | SEQ ID NO: 20 |
| 22 | TNS1 | | Adaptor/scaffold | Y1326 | HVAYGGySTPEDR | SEQ ID NO 21 |
| 23 | TRPC4AP | NP_056453.1 | Adaptor/scaffold | Y603 | FNKyINTDAKFQVFLKQINSSLVDSNML | SEQ ID NO: 22 |
| | | | | | VR | |
| 24 | LPP | NP_005569.1 | Adaptor/scaffold; | Y273 | GGMDyAYIPPPGLQPEPGYGYAPNQGR | SEQ ID NO: 23 |
| | | | Cytoskeletal protein | | | |
| 25 | FNBP1L | NP_060207.2 | Adaptor/scaffold; | Y448 | ESPEGSyTDDANQEVR | SEQ ID NO: 24 |
| | | | Unknown function | | | |
| 26 | EPS15L1 | | Adaptor/scaffold; | Y564 | SLEQyDQVLDGAHGASLTDLANLSEGVS | SEQ ID NO. 25 |
| | | | Vesicle protein | | LAER | |
| 27 | CDH3 | NP_001784.2 | Adhesion | Y713 | DNVFYYGEEGGGEEOQDyDITQLHR | SEQ ID NO: 26 |
| 28 | CDH3 | NP_001784.2 | Adhesion | Y823 | KLADMyGGGEDD | SEQ ID NO: 27 |
| 29 | CDH6 | NP_004923.1 | Adhesion | Y4 | TyRYFLLLFWVGQPYPTLSTPLSK | SEQ ID NO: 28 |
| 30 | CDH6 | NP_004923.1 | Adhesion | Y6 | TYRyFLLLFWVGQPYPTLSTPLSK | SEQ ID NO: 29 |
| 31 | DCBLD2 | NP_563615.3 | Adhesion | Y565 | KTEGTyDLPYWDR | SEQ ID NO: 30 |
| 32 | DSC3 | NP_001932.1 | Adhesion | Y493 | IKENLAVGSKINGyK | SEQ ID NO: 31 |
| 33 | ERBB2IP | NP_00100660 | Adhesion | Y1021 | SESTENQSyAKHSANMNFSNHNNVR | SEQ ID NO: 32 |
| | | 0.1 | | | | |
| 34 | F11R | NP_058642.1 | Adhesion | Y280 | KVIySQPSAR | SEQ ID NO: 33 |
| 35 | HSPG2 | CAA44373.1 | Adhesion | Y1711 | GPHYFyWSREDGRPVPSGTQQR | SEQ ID NO: 34 |
| 36 | ITGA2 | NP_002194.1 | Adhesion | Y1005 | NPLMyLTGVQTDKAGDISCNADINPLKIG | SEQ ID NO: 35 |
| | | | | | QTSSSVSFK | |
| 37 | ITGAM | NP_000623.2 | Adhesion | Y283 | EGVIRyVIGVGDAFRSEK | SEQ ID NO: 36 |
| 38 | ITGBS | NP_002204.2 | Adhesion | Y774 | ARYEMASNPLyR | SEQ ID NO: 37 |
| 39 | L1CAM | NP_076493.1 | Adhesion | Y1151 | ySVKDKEDTQVDSEARPMKDETFGEYS | SEQ ID NO: 38 |
| | | | | | DNEEK | |
| 40 | LAMA4 | NP_002281.1 | Adhesion | Y1317 | yELIVDKSR | SEQ ID NO: 39 |
| 41 | MCAM | NP_006491.2 | Adhesion | Y641 | APGDQGEKyIDLRH | SEQ ID NO: 40 |
| 42 | NRXN2 | NP_055895.1 | Adhesion | Y41 | yARWAGAASSGELSFSLRTNATR | SEQ ID NO: 41 |
| 43 | OCLN | NP_002529.1 | Adhesion | Y287 | SNILWDKEHIyDEQPPNVEEWVK | SEQ ID NO: 42 |
| 44 | OCLN | NP_002529.1 | Adhesion | Y315 | NVSAGTQDVPSPPSDyVERVDSPMAYS | SEQ ID NO: 43 |
| | | | | | SNGK | |
| 45 | OCLN | NP_002529.1 | Adhesion | Y402 | TEQDHYETDyTTGGESCDELEEDWIR | SEQ ID NO: 44 |
| 46 | OCLN | NP_002529.1 | Adhesion | Y443 | NFDTGLQEyK | SEQ ID NO: 45 |
| 47 | PCDH1 | NP115796.2 | Adhesion | Y1058 | LQDPSQHSyYDSGLEE | SEQ ID NO: 46 |
| 48 | PCDH20 | NP_073754.1 | Adhesion | Y883 | VESVSCMPTLVALSVISLGSITLVTGMGIy | SEQ ID NO: 47 |
| | | | | | ICLRK | |
| 49 | PCDHB15 | NP_061758.1 | Adhesion | Y279 | DLDTGTNGEISySLYYSSQEIDK | SEQ ID NO: 48 |
| 50 | PCDHB15 | NP_061758.1 | Adhesion | Y282 | DLDTGTNGEISYSLyYSSQEIDK | SEQ ID NO: 49 |
| 51 | PCDHB15 | NP_061758.1 | Adhesion | Y283 | DLDTGTNGEISYSLYySSQEIDK | SEQ ID NO: 50 |
| 52 | PKP3 | NP_009114.1 | Adhesion | Y390 | NLIyDNADNK | SEQ ID NO: 51 |
| 53 | PVRL4 | NP_112178.1 | Adhesion | Y502 | KPTGNGIyINGR | SEQ ID NO: 52 |
| 54 | DSG2 | NP_001934.1 | Adhesion; Calcium- | Y967 | VyAPASTLVDQPYANEGTVVVTER | SEQ ID NO: 53 |
| | | | binding protein | | | |
| 55 | DSG2 | NP_001934.1 | Adhesion; Calcium- | Y978 | VYAPASTLVDQPyANEGTVVVTER | SEQ ID NO: 54 |
| | | | binding protein | | | |
| 56 | DSG2 | NP_001934.1 | Adhesion; Calcium- | Y1060 | VLAPASTLQSSyQIPTENSMTAR | SEQ ID NO: 55 |
| | | | binding protein | | | |
| 57 | PTPNS1 | NP542970.1 | Adhesion; Cell surface; | Y429 | EITQDTNDITyADLNLPK | SEQ ID NO: 56 |
| | | | Receptor, misc. | | | |
| 58 | IFIH1 | NP_071451.2 | Apoptosis | Y1000 | KQyKKWVELPITFPNLDYSECCLFSDED | SEQ ID NO: 57 |
| 59 | IFIH1 | NP_071451.2 | Apoptosis | Y1015 | KQYKKWVELPITFPNLDySECCLFSDED | SEQ ID NO: 58 |
| 60 | MAEA | NP_00101740 | Apoptosis | Y19 | MTLKVQEyPTLKVPYETLNKR | SEQ ID NO: 59 |
| | | 5.1 | | | | |
| 61 | LLGL2 | NP_004515.2 | Cell cycle regulation | Y499 | VGSFDPySDDPR | SEQ ID NO: 60 |
| 62 | MSH4 | NP_002431.2 | Cell cycle regulation | Y889 | AVyHLATRLVQTAR | SEQ ID NO: 61 |
| 63 | SYCP2 | NP_055073.2 | Cell cycle regulation | Y1453 | EFVDFWEKIFQKFSAyQK | SEQ ID NO: 62 |
| 64 | TACC2 | NP_008928.1 | Cell cycle regulation | Y804 | EAAHPTDVSISKTALySR | SEQ ID NO: 63 |
| 65 | CSPG6 | | Cell cycle regulation; | Y669 | GALTGGYyDTR | SEQ ID NO: 64 |
| | | | DNA repair | | | |
| 66 | HEM1 | NP_056416.2 | Cell surface | Y315 | VTEDLFSSLKGyGKRVADIK | SEQ ID NO: 65 |
| 67 | KM-HN-1 | NP689988.1 | Cell surface | Y790 | ICNQHNDPSKTTyISR | SEQ ID NO: 66 |
| 68 | M11S1 | NP_005889.3 | Cell surface | Y449 | GYTASQPLyQPSHATE | SEQ ID NO: 67 |
| 69 | MUC13 | | Cell surface | Y500 | DSQMQNPySR | SEQ ID NO: 68 |
| 70 | MUC13 | NP_149038.2 | Cell surface | Y511 | HSSMPRPDy | SEQ ID NO:69 |
| 71 | ROM1 | NP_000318.1 | Cell surface | Y288 | yLQTALEGLGGVIDAGGETQGYLFPSG | SEQ ID NO: 70 |
| | | | | | LK | |
| 72 | ROM1 | NP_000318.1 | Cell surface | Y309 | LQTALEGLGGVIDAGGETQGyLFPSG | SEQ ID NO: 71 |
| | | | | | LK | |
| 73 | SLITRK6 | NP_115605.2 | Cell surface | Y805 | LMETLMySRPR | SEQ ID NO: 72 |
| 74 | SLITRK6 | NP_115605.2 | Cell surface | Y820 | KVLVEQTKNEyFELK | SEQ ID NO: 73 |
| 75 | RYR3 | NP_001027.2 | Channel, calcium | Y2824 | LEDDPLyTSYSSMMAK | SEQ ID NO: 74 |
| 76 | CLCN1 | NP_000074.1 | Channel, chloride | Y686 | LRAAQEMARKLSELPyDGKAR | SEQ ID NO: 75 |
| 77 | GJA1 | NP_000156.1 | Channel, misc. | Y313 | QASEQNWANySAEQNR | SEQ ID NO: 76 |
| 78 | KCNQ3 | NP_004510.1 | Channel, potassium | Y502 | GyGNDFPIEDMIPTLK | SEQ ID NO: 77 |
| 79 | TBCE | NP_003184.1 | Chaperone | Y493 | LLKVPVSDLLLSyESPKK | SEQ ID NO: 78 |
| 80 | EPB41L1 | NP_036288.2 | Cytoskeletal protein | Y864 | AVVyRETDPSPEER | SEQ ID NO 79 |
| 81 | EPB41L4A | NP_071423.3 | Cytoskeletal protein | Y576 | EELWKHIQKELVDPSGLSEEQLKEIPyTK | SEQ ID NO. 80 |
| 82 | HOOK2 | NP_037444.1 | Cytoskeletal protein | Y603 | yVDKARMVMQTMEPK | SEQ ID NO: 81 |
| 83 | KRT12 | NP_000214.1 | Cytoskeletal protein | Y262 | TDLEMQIESLNEELAyMK | SEQ ID NO: 82 |
| 84 | KRT20 | NP_061883.1 | Cytoskeletal protein | Y384 | TTEyQLSTLEER | SEQ ID NO: 83 |
| 85 | KRT2A | NP_000414.2 | Cytoskeletal protein | Y268 | yEDEINKRTAAENDFVTLK | SEQ ID NO: 84 |
| 86 | KRTHB2 | NP_149022.3 | Cytoskeletal protein | Y451 | GAFLyEPCGVSTPVLSTGVLR | SEQ ID NO: 85 |
| 87 | SMTN | NP_599031.1 | Cytoskeletal protein | Y896 | EPDWKCVYTyIQEFYR | SEQ ID NO: 86 |
| 88 | SMTN | NP_599031.1 | Cytoskeletal protein | Y901 | EPDWKCVYTYIQEFyR | SEQ ID NO: 87 |
| 89 | SPTA1 | NP_003117.1 | Cytoskeletal protein | Y2304 | GLNyYLPMVEEDEHEPKFEK | SEQ ID NO: 88 |
| 90 | SPTBN2 | NP_008877.1 | Cytoskeletal protein | Y604 | EyRPCDPQLVSERVAK | SEQ ID NO: 89 |
| 91 | SPTBN4 | NP_066022.1 | Cytoskeletal protein | Y2457 | SWVSLYCVLSKGELGFyKDSK | SEQ ID NO: 90 |
| 92 | TUBA3 | NP_006000.2 | Cytoskeletal protein | Y432 | EDMAALEKDyEEVGVDSVEGEGEEEGE | SEQ ID NO: 91 |
| | | | | | EY | |
| 93 | TUBA6 | NP_116093.1 | Cytoskeletal protein | Y449 | DYEEVGADSADGEDEGEEy | SEQ ID NO: 92 |
| 94 | PXN | | Cytoskeletal protein, | Y76 | yAHQQPPSPLPVYSSSAK | SEQ ID NO: 93 |
| | | | Apoptosis | | | |
| 95 | FLJ11806 | NP_079100.2 | DNA binding protein | Y273 | LCEPEVLNSLEETySPFFR | SEQ ID NO: 94 |
| 96 | SMARCA5 | NP_003592.2 | DNA binding protein | Y719 | LSKMGESSLRNFTMDTESSVYNFEGEDyR | SEQ ID NO: 95 |
| 97 | SON | NP_115571.1 | DNA binding protein | Y909 | LGQDPyRLGHDPYR | SEQ ID NO: 96 |
| 98 | ZBED1 | NP_004720.1 | DNA binding protein | Y479 | EVIAKELSKTYQETPEIDMFLNVATFLDP | SEQ ID NO: 97 |
| | | | | | RyK | |
| 99 | CRY1 | NP_004066.1 | DNA binding protein; | Y266 | LFyFKLTDLYKKVK | SEQ ID NO: 98 |
| | | | Lyase | | | |
| 100 | ERCC6 | NP_000115.1 | DNA repair | Y1279 | HDAIMDGASPDyVLVEAEANRVAQDALK | SEQ ID NO: 99 |
| 101 | POLI | NP_009126.1 | DNA repair | Y377 | LGTGNyDVMTPMVDILMK | SEQ ID NO: 100 |
| 102 | MCM4 | NP_005905.2 | DNA replication | Y730 | IGSSRGMVSAyPR | SEQ ID NO: 101 |
| 103 | POLA | NP_058633.2 | DNA replication | Y1430 | QFFTPKVLQDyR | SEQ ID NO: 102 |
| 104 | SMC5L1 | NP_055925.1 | DNA replication | Y626 | YWKTSFySNK | SEQ ID NO: 103 |
| 105 | CTPS | NP_001896.1 | Enzyme, misc. | Y473 | LYGDADyLEER | SEQ ID NO: 104 |
| 106 | DPYD | NP_000101.1 | Enzyme, misc. | Y882 | IAELMDKKLPSFGPyLEQRKK | SEQ ID NO: 105 |
| 107 | ENTPD1 | NP_001767.3 | Enzyme, misc. | Y287 | DPCFHPGyKKVVNVSDLYKTPCTK | SEQ ID NO: 106 |
| 108 | GLCE | NP_056369.1 | Enzyme, misc. | Y477 | DHIFLNSALRATAPyK | SEQ ID NO: 107 |
| 109 | GLULD1 | NP_057655.1 | Enzyme, misc. | Y490 | yELENEEIAAERNK | SEQ ID NO: 108 |
| 110 | GPAA1 | NP_003792.1 | Enzyme, misc. | Y328 | VEALTLRGINSFRQyKYDLVAVGKALEG | SEQ ID NO: 109 |
| | | | | | MFR | |
| 111 | GPAA1 | NP_003792.1 | Enzyme, misc. | Y330 | VEALTLRGINSFRQYKyDLVAVGKALEG | SEQ ID NO: 110 |
| | | | | | MFR | |
| 112 | NAGLU | NP_000254.2 | Enzyme, misc. | Y92 | VRGSTGVAMAGLHRyLR | SEQ ID NO: 111 |
| 113 | PYGM | NP_005600.1 | Enzyme, misc. | Y473 | DFyELEPHKFQNKTNGITPR | SEQ ID NO: 112 |
| 114 | TKTL1 | NP_036385.2 | Enzyme, misc. | Y112 | RLSFVDVATGWLGQGLGVACGMAYTGK | |
| | | | | | yFDR | SEQ ID NO: 113 |
| 115 | UMPS | NP_000364.1 | Enzyme, misc. | Y37 | SGLSSPIyIDLR | SEQ ID NO: 114 |
| 116 | VARS | NP_006286.1 | Enzyme, misc. | Y469 | LHEEGIIyR | SEQ ID NO: 115 |
| 117 | COL11A1 | NP 542196.2 | Extracellular matrix | Y329 | AKLGVKANIVDDFQEYNYGTMESyQTEA | SEQ ID NO: 116 |
| | | | | | PR | |
| 118 | COL16A1 | NP_001847.3 | Extracellular matrix | Y1108 | GERGyTGSAGEKGEPGPPGSEGLPGPP | SEQ ID NO: 117 |
| | | | | | GPAGPRGER | |
| 119 | FRAS1 | NP_079350.4 | Extracellular matrix | Y2722 | GDASSIVSAICYTVPKSAMGSSLyALESG | SEQ ID NO: 118 |
| | | | | | SDFKSR | |
| 120 | TLL2 | NP_036597.1 | Extracellular matrix | Y541 | DGPTEESALIGHFCGyEK | SEQ ID NO: 119 |
| 121 | TNXB | NP_061978.5 | Extracellular matrix | Y1183 | WTVPEGEFDSFVIQyKDR | SEQ ID NO: 120 |
| 122 | GDI2 | NP_001485.2 | G protein regulator, | Y333 | KSDIyVCMISFAHNVAAQGK | SEQ ID NO: 121 |
| | | | misc. | | | |
| 123 | GDI2 | NP_001485.2 | G protein regulator, | Y442 | MKRKKNDIyGED | SEQ ID NO: 122 |
| | | | misc. | | | |
| 124 | DDEF2 | NP_003878.1 | GTPase activaing | Y763 | AFMPSILQNETyGALLSGSPPPAQPAAP | SEQ ID NO: 123 |
| | | | protein, ARF | | STTSAPPLPPR | |
| 125 | RICS | NP_055530.2 | GTPase activating | Y1208 | VEyVSSLSSSVR | SEQ ID NO: 124 |
| | | | protein, Rac/Rho | | | |
| 126 | RICS | NP_055530.2 | GTPase activating | Y1557 | QFCESKNGPPYPQGAGQLDyGSK | SEQ ID NO: 125 |
| | | | protein, Rac/Rho | | | |
| 127 | RICS | NP_055530.2 | GTPase activating | Y1680 | QSSVTWSQYDNLEDyHSLPQHQR | SEQ ID NO: 126 |
| | | | protein, Rac/Rho | | | |
| 128 | NF1 | NP_000258.1 | GTPase activaing | Y2556 | RVAETDyEMETQR | SEQ ID NO: 127 |
| | | | protein, Ras | | | |
| 129 | RALGPS2 | NP_689876.2 | Guanine nucleotide | Y420 | NRLyHSLGPVTR | SEQ ID NO: 128 |
| | | | exchange factor, Ras | | | |
| 130 | RASGRP3 | NP_733772.1 | Guanine nucleotide | Y523 | QGyKCKDCGANCHKQCKDLLVLACR | SEQ ID NO: 129 |
| | | | exchange factor, Ras | | | |
| 131 | DDX6 | NP_004388.1 | Helicase | Y462 | SLYVAEyHSEPVEDEKP | SEQ ID NO: 130 |
| 132 | NAV2 | NP_660093.2 | Helicase | Y1179 | KSSMDGAQNQDDGyLALSSR | SEQ ID NO: 131 |
| 133 | NAV2 | NP_660093.2 | Helicase | Y1579 | THSLSNADGQYDPyTDSRFR | SEQ ID NO: 132 |
| 134 | THEA | NP_056362.1 | Hydrolase, esterase | Y364 | YREASARKKIRLDRKyIVSCK | SEQ ID NO: 133 |
| 135 | LEMD3 | NP_055134.2 | Inhibitor protein | Y667 | EEEETRQMyDMWKLIDVLR | SEQ ID NO: 134 |
| 136 | MIG-6 | NP_061821.1 | Inhibitor protein | Y341 | SLPSyLNGVMPPTQSFAPDPK | SEQ ID NO: 135 |
| 137 | MIG-6 | NP_061821.1 | Inhibitor protein | Y358 | SLPSYLNGVMPPTQSFAPDPKyVSSK | SEQ ID NO: 136 |
| 138 | HK2 | NP_000180.2 | Kinase (non-protein) | Y301 | TEFDQEIDMGSLNPGKQLFEKMISGMyM | SEQ ID NO: 137 |
| | | | | | GELVR | |
| 139 | PIK3CB | NP_006210.1 | Kinase, lipid | Y436 | TINPSKYQTIRKAGKVHyPVAWVNTMVF | SEQ ID NO: 138 |
| | | | DFK | | | |
| 140 | PIK3CD | NP_005017.2 | Kinase, lipid | Y440 | CLyMWPSVPDEKGELLNPTGTVR | SEQ ID NO: 139 |
| 141 | PIK4CA | NP_477352.1 | Kinase, lipid | Y470 | LYKYHSQyHTVAGNDIK | SEQ ID NO: 140 |
| 142 | PIK4CA | NP_477352.1 | Kinase, lipid | Y1096 | NRYAGEVyGMIR | SEQ ID NO: 141 |
| 143 | PIP5K1A | NP_003548.1 | Kinase, lipid | Y470 | GSSGNSCITyQPSVSGEHK | SEQ ID NO: 142 |
| 144 | TTK | NP_003309.2 | KINASE; Protein | Y374 | LEETKEyQEPEVPESNQK | SEQ ID NO: 143 |
| | | | kinase, dual-specificity | | | |
| 145 | LMTK2 | NP_055731.2 | KINASE; Protein | Y1468 | STEQSWPHSAPySR | SEQ ID NO: 144 |
| | | | kinase, Ser/Thr | | | |
| 146 | ILK | NP_00101479 | KINASE; Protein | Y351 | MyAPAWVAPEALQK | SEQ ID NO: 145 |
| | | 4.1 | kinase, Ser/Thr (non- | | | |
| | | | receptor) | | | |
| 147 | IRAK1 | NP_001560.2 | KINASE; Protein | Y395 | TQTVRGTLAYLPEEyIKTGR | SEQ ID NO: 146 |
| | | | kinase, Ser/Thr (non- | | | |
| | | | receptor) | | | |
| 148 | MAP4K5 | NP_006566.2 | KINASE; Protein | Y401 | ISSyPEDNFPDEEK | SEQ ID NO: 147 |
| | | | kinase, Ser/Thr (non- | | | |
| | | | receptor) | | | |
| 149 | NRK | NP_940867.1 | KINASE; Protein | Y984 | FVDDVNNNyYEAPSCPR | SEQ ID NO: 148 |
| | | | kinase, Ser/Thr (non- | | | |
| | | | receptor) | | | |
| 150 | TLK1 | NP_036422.3 | KINASE; Protein | Y481 | yAAVKIHQLNKSWRDEK | SEQ ID NO: 149 |
| | | | kinase, Ser/Thr (non- | | | |
| | | | receptor) | | | |
| 151 | TTN | NP_003310.3 | KINASE; Protein | Y1713 | LRMINEFGyCSLDYGVAYSR | SEQ ID NO: 150 |
| | | | kinase, Ser/Thr (non- | | | |
| | | | receptor) | | | |
| 152 | TTN | NP_003310.3 | KINASE; Protein | Y1981 | DESyEELLRKTK | SEQ ID NO: 151 |
| | | | kinase, Ser/Thr (non- | | | |
| | | | receptor) | | | |
| 153 | KIAA2002 | XP_940171.1 | KINASE; Protein | Y387 | EIEPNyESPSSNNQDKDSSQASK | SEQ ID NO: 152 |
| | | | kinase, Ser/Thr (non- | | | |
| | | | receptor, predicted) | | | |
| 154 | KIAA2002 | XP_940171.1 | KINASE; Protein | Y531 | SSAIRyQEVWTSSTSPR | SEQ ID NO: 153 |
| | | | kinase, Ser/Thr (non- | | | |
| | | | receptor, predicted) | | | |
| 155 | KIAA2002 | XP_940171.1 | KINASE; Protein | Y635 | NAIKVPIVINPNAyDNLAIYK | SEQ ID NO: 154 |
| | | | kinase, Ser/Thr (non- | | | |
| | | | receptor, predicted) | | | |
| 156 | KIAA2002 | | KINASE; Protein | Y641 | NAIKVPIVINPNAYDNLAIyK | SEQ ID NO: 155 |
| | | | kinase, Ser/Thr (non- | | | |
| | | | receptor, predicted) | | | |
| 157 | KIAA2002 | XP_940171.1 | KINASE; Protein | Y665 | TTSVISHTyEEIETESK | SEQ ID NO: 156 |
| | | | kinase, Ser/Thr (non- | | | |
| | | | receptor, predicted) | | | |
| 158 | KIAA2002 | XP_940171.1 | KINASE; Protein | Y797 | CSVEELyAIPPDADVAK | SEQ ID NO: 157 |
| | | | kinase, Ser/Thr (non- | | | |
| | | | receptor, predicted) | | | |
| 159 | KIAA2002 | XP_940171.1 | KINASE; Protein | Y880 | STSSPyHAGNLLQR | SEQ ID NO: 158 |
| | | | kinase, Ser/Thr (non | | | |
| | | | receptor, predicted) | | | |
| 160 | TNK1 | NP_003976.1 | KINASE; Protein | Y661 | ILEHYQWOLSAASRyVLARP | SEQ ID NO: 159 |
| | | | kinase, tyrosine (non- | | | |
| | | | receptor) | | | |
| 161 | EPHA1 | NP_005223.3 | KINASE; Receptor | Y781 | LLDDFDGTyETQGGK | SEQ ID NO: 160 |
| | | | tyrosine kinase | | | |
| 162 | EPHB3 | NP_004434.2 | KINASE; Receptor | Y600 | LQQyIAPGMK | SEQ ID NO: 161 |
| | | | tyrosine kinase | | | |
| 163 | EPHB4 | NP_004435.3 | KINASE; Receptor | Y906 | QPHySAFGSVGEWLR | SEQ ID NO: 162 |
| | | | tyrosine kinase | | | |
| 164 | FLT1 | NP_002010.1 | KINASE; Receptor | Y1048 | DIyKNPDYVR | SEQ ID NO: 163 |
| | | | tyrosine kinase | | | |
| 165 | TIE1 | NP_005415.1 | KINASE; Receptor | Y969 | QLLRFASDAANGMQyLSEKQFIHR | SEQ ID NO: 164 |
| | | | tyrosine kinase | | | |
| 166 | PLEKHA5 | NP_061885.2 | Lipid binding protein | Y398 | GGNRPNTGPLyTEADR | SEQ ID NO: 165 |
| 167 | PRODH | NP_057419.2 | Mitochondrial | Y412 | PLIFNTyQCYLKDAYDNVTLDVELARR | SEQ ID NO: 166 |
| 168 | PRSS15 | NP_004784.2 | Mitochondrial | Y394 | yLLQEQLKIIK | SEQ ID NO: 167 |
| 169 | SLC25A1 | NP_005975.1 | Mitochondrial | Y276 | YRNTWDCGLQILKKEGLKAFyK | SEQ ID NO: 168 |
| 170 | SLC25A5 | NP_001143.1 | Mitochondrial | Y191 | AAYFGIyDTAK | SEQ ID NO: 169 |
| 171 | TOP1MT | NP_443195.1 | Mitochondrrial | Y455 | ILSyNRANRWAILCNHQR | SEQ ID NO: 170 |
| 172 | DNCH1 | NP_001367.2 | Motor protein | Y3379 | KNYMSNPSYNyEIVNR | SEQ ID NO: 171 |
| 173 | KIFlA | NP_004312.2 | Motor protein | Y1666 | DMHDWLyAFNPLLAGTIRSK | SEQ ID NO: 172 |
| 174 | KIF2B | NP_115948.3 | Motor protein | Y536 | yANRVKKLNVDVR | SEQ ID NO: 173 |
| 175 | MYH1 | NP_005954.2 | Motor protein | Y820 | ESIFCIQyNVR | SEQ ID NO: 174 |
| 176 | MYH10 | NP_005955.1 | Motor protein | Y285 | TFHIFyQLLSGAGEHLK | SEQ ID NO: 175 |
| 177 | MYH13 | NP_003793.2 | Motor protein | Y1351 | HDCDLLREQyEEEQEAK | SEQ ID NO: 176 |
| 178 | MYH2 | NP_060004.2 | Motor protein | Y413 | ALCYPRVKVGNEyVTKGQTVEQVSNAV | SEQ ID NO: 177 |
| | | | | | GALAKAVYEK | |
| 179 | MYH3 | NP_002461.2 | Motor protein | Y284 | SyHIFYQILSNK | SEQ ID NO: 178 |
| 180 | MYH3 | NP_002461.2 | Motor protein | Y288 | SYHIFyQILSNK | SEQ ID NO: 179 |
| 181 | MYH4 | NP_060003.2 | Motor protein | Y389 | AAyLTSLNSADLLK | SEQ ID NO: 180 |
| 182 | MYH8 | NP_002463.1 | Motor protein | Y1463 | QKyEETQAELEASQK | SEQ ID NO: 181 |
| 183 | MYH8 | NP_002463.1 | Motor protein | Y1855 | ELTyQTEEDRK | SEQ ID NO: 182 |
| 184 | MYO1D | NP_056009.1 | Motor protein | Y885 | HLyKMDPTKQYKVMKTIPLYNLTGLSVSN | SEQ ID NO: 183 |
| | | | | | GK | |
| 185 | MYO1D | NP_056009.1 | Motor protein | Y893 | HLYKMDPTKQyKVMKTIPLYNLTGLSVSN | SEQ ID NO: 184 |
| | | | | | GK | |
| 186 | MYO1D | NP_056009.1 | Motor protein | Y902 | HLYKMDPTKQYKVMKTIPLyNLTGLSVSN | SEQ ID NO: 185 |
| | | | | | GK | |
| 187 | MYO1E | NP_004989.2 | Motor protein | Y971 | NQyVPYPHAPGSQR | SEQ ID NO: 186 |
| 188 | MYO1E | NP_004989.2 | Motor protein | Y989 | SLyTSMARPPLPR | SEQ ID NO: 187 |
| 189 | MYO5A | NP_000250.1 | Motor protein | Y834 | yKIRRAATIVLQSYLR | SEQ ID NO: 188 |
| 190 | MYO5B | XP_371116.4 | Motor protein | Y1046 | VEyLSDGFLEKNR | SEQ ID NO: 189 |
| 191 | MYBPC2 | NP_004524.2 | Myosin binding protein | Y1003 | HTSCTVSDLIVGNEYyFR | SEQ ID NO: 190 |
| 192 | PPP2R5C | NP_002710.2 | Phosphatase, | Y443 | NPQyTVYSQASTMSIPVAMETDGPLFE | SEQ ID NO: 191 |
| | | | regulatory subunit | | DVQMLRK | |
| 193 | PHLPP | NP_919431.1 | PHOSPHATASE; | Y1200 | HYQLDQLPDyYDTPL | SEQ ID NO: 192 |
| | | | Protein phosphatase, | | | |
| | | | Ser/Thr (non-receptor) | | | |
| 194 | PPP1CA | NP_00100870 | PHOSPHATASE; | Y317 | yGQFSGLNPGGRPITPPR | SEQ ID NO: 193 |
| | | 9.1 | Protein phosphatase, | | | |
| | | | Ser/Thr (non-receptor) | | | |
| 195 | PTPN11 | NP_002825.3 | PHOSPHATASE; | Y263 | LLySRKEGQRQENKNK | SEQ ID NO: 194 |
| | | | Protein phosphatase, | | | |
| | | | tyrosine (non-receptor) | | | |
| 196 | PTPRS | NP_570923.2 | PHOSPHATASE; | Y205 | yECVATNSAGVRYSSPANLYVRVR | SEQ ID NO: 195 |
| | | | Receptor protein | | | |
| | | | phosphatase, tyrosine | | | |
| 197 | PTPRT | NP_008981.3 | PHOSPHATASE; | Y345 | TTTGTWAETHIVDSPNyK | SEQ ID NO: 196 |
| | | | Receptor protein | | | |
| | | | phosphatase, tyrosine | | | |
| 198 | PTPRT | NP_008981.3 | PHOSPHATASE; | Y1003 | CVRyWPDDTEVYGDIK | SEQ ID NO: 197 |
| | | | Receptor protein | | | |
| | | | phosphatase, tyrosine | | | |
| 199 | PTPRT | NP_008981.3 | PHOSPHATASE; | Y1011 | YWPDDTEVyGDIKVTLIETEPLAEYVIRTF | SEQ ID NO: 198 |
| | | | Receptor protein | | TVQK | |
| | | | phosphatase, tyrosine | | | |
| 200 | TPTE | NP_954870.2 | PHOSPHATASE; | Y509 | LyLPKNELDNLHKQK | SEQ ID NO: 199 |
| | | | Receptor protein | | | |
| | | | phosphatase, tyrosine | | | |
| 201 | PDE6C | NP_006195.2 | Phosphodiesterase | Y277 | SYLNCERySIGLLDMTK | SEQ ID NO: 200 |
| 202 | PLCG1 | NP_002651.2 | Phospholipase | Y977 | CyRDMSSFPETK | SEQ ID NO: 201 |
| 203 | CPD | NP_001295.2 | Protease (non- | Y520 | FANEyPNITRLYSLGKSVESR | SEQ ID NO: 202 |
| | | | proteasomal) | | | |
| 204 | CPD | NP_001295.2 | Protease (non- | Y1344 | LRQHHDEyEDEIR | SEQ ID NO: 203 |
| | | | poroteasomal) | | | |
| 205 | CPD | NP_001295.2 | Protease (non- | Y1376 | SLLSHEFQDETDTEEETLySSKH | SEQ ID NO: 204 |
| | | | proteasomal) | | | |
| 206 | MMP15 | NP_002419.1 | Protease (non- | Y525 | PISVWQGIPASPKGAFLSNDAAyTYFYKG | SEQ ID NO: 205 |
| | | | proteasomal) | | TK | |
| 207 | MMP15 | NP_002419.1 | Protease (non- | Y527 | PISVWQG IPASPKGAFLSNDAAYTyFYKG | SEQ ID NO: 206 |
| | | | proteasomal) | | TK | |
| 208 | NAALADL2 | NP_996898.1 | Protease (non- | Y110 | LQEESDYITHyTR | SEQ ID NO: 207 |
| | | | proteasomal) | | | |
| 209 | SENP6 | NP_056386.1 | Protease (non- | Y781 | yEPNPHYHENAVIQK | SEQ ID NO: 208 |
| | | | proteasomal) | | | |
| 210 | YME1L1 | NP_055078.1 | Protease (non- | Y646 | FGMSEKLGVMTySDTGK | SEQ ID NO: 209 |
| | | | proteasomal) | | | |
| 211 | F2R | NP_001983.1 | Receptor, GPCR | Y420 | MDTCSSNLNNSIyK | SEQ ID NO: 210 |
| 212 | GABBR1 | NP_001461.1 | Receptor, GPCR | Y776 | KMNTWLGIFYGyK | SEQ ID NO: 211 |
| 213 | LPHN2 | NP_036434.1 | Receptor, GPCR | Y1350 | RSENEDIyYK | SEQ ID NO: 212 |
| 214 | OR2D3 | NP_00100468 | Receptor, GPCR | Y294 | ELDKMISVFyTAVTPMLNPIIYSLR | SEQ ID NO: 213 |
| | | 4.1 | | | | |
| 215 | OR2D3 | NP_00100468 | Receptor, GPCR | Y306 | ELDKMISVFYTAVTPMLNPIIySLR | SEQ ID NO: 214 |
| | | 4.1 | | | | |
| 216 | OR7G1 | NP_00100519 | Receptor, GPCR | Y278 | ITAVASVMyTVVPQMMNPFIYSLR | SEQ ID NO: 215 |
| | | 2.1 | | | | |
| 217 | | BAC45258.1 | Receptor, GPCR | Y475 | yLGIMKPLTYPMRQK | SEQ ID NO: 216 |
| 218 | IGF2R | NP_000867.1 | Receptor, misc. | Y1834 | TySVGVCTFAVGPEQGGCKDGGVCLLS | SEQ ID NO: 217 |
| | | | | | GTKGASFGR | |
| 219 | LRP1B | NP_061027.2 | Receptor, misc. | Y1708 | LyWTDGNTINMANMDGSNSKILFQNQK | SEQ ID NO: 218 |
| 220 | LRP6 | NP_002327.1 | Receptor, misc. | Y1584 | SQYLSAEENyESCPPSPYTER | SEQ ID NO: 219 |
| 221 | NEO1 | NP_002490.1 | Receptor, misc. | Y548 | AyAASPTSITVTWETPVSGNGEIQNYK | SEQ ID NO: 220 |
| 222 | NEO1 | NP_002490.1 | Receptor, misc. | Y572 | YAASPTSITVTWETPVSGNGEIQNyK | SEQ ID NO: 221 |
| 223 | NRP1 | NP_003864.3 | Receptor, misc. | Y920 | DKLNTQSTySEA | SEQ ID NO: 222 |
| 224 | NRP2 | NP_003863.2 | Receptor, misc. | Y720 | SPVCMEFQyQATGGRGVALQVVR | SEQ ID NO: 223 |
| 225 | ODZ2 | XP_047995.9 | Receptor, misc. | Y1601 | YYLAVDPVSGSLYVSDTNSRRIyRVK | SEQ ID NO: 224 |
| 226 | ODZ3 | XP_371717.3 | Receptor, misc. | Y1479 | HAVQTTLESATAIAVSYSGVLyITETDEKK | SEQ ID NO: 225 |
| 227 | ODZ4 | | Receptor, misc. | Y2547 | TWSYTYLEKAGVCLPASLALPyR | SEQ ID NO: 226 |
| 228 | ODZ4 | XP_166254.6 | Receptor, misc. | Y3071 | QILYTAYGEIyMDTNPNFQIIIGYHGGLYD | SEQ ID NO: 227 |
| | | | | | PLTK | |
| 229 | PEAR1 | XP_371320.3 | Receptor, misc. | Y1251 | DLPSLPGGPRESSyMEMK | SEQ ID NO: 228 |
| 230 | PLXNA1 | NP_115618.2 | Receptor, misc. | Y1585 | QTSAyNISNSSTFTK | SEQ ID NO: 229 |
| 231 | PLXNC1 | NP_005752.1 | Receptor, misc. | Y1350 | EMyLTKLLSTKVAIHSVLEK | SEQ ID NO: 230 |
| 232 | PLXND1 | NP_055918.1 | Receptor, misc. | Y1642 | KLNTLAHyKIPEGASLAMSLIDKK | SEQ ID NO: 231 |
| 233 | SDC1 | NP_00100694 | Receptor, misc. | Y286 | KKDEGSySLEEPK | SEQ ID NO: 232 |
| | | 7.1 | | | | |
| 234 | SDC1 | NP_00100694 | Receptor, misc. | Y299 | QANGGAyQKPTKQEEFYA | SEQ ID NO: 233 |
| | | 7.1 | | | | |
| 235 | SDC3 | NP_055469.2 | Receptor, misc. | Y441 | QASVTYQKPDKQEEFyA | SEQ ID NO: 234 |
| 236 | SIGIRR | NP_068577.1 | Receptor, misc. | Y395 | SSEVDVSDLGSRNySAR | SEQ ID NO: 235 |
| 237 | SLAMF6 | NP_443163.1 | Receptor, misc. | Y308 | ENDTITIySTINHSK | SEQ ID NO: 236 |
| 238 | TLR10 | NP_00101738 | Receptor, misc. | Y786 | EMyELQTFTELNEESR | SEQ ID NO: 237 |
| | | 8.1 | | | | |
| 239 | SLC20A2 | NP_006740.1 | Receptor, misc.; | Y354 | DSGLyKDLLHK | SEQ ID NO: 238 |
| | | | Transporter, facilitator | | | |
| 240 | 2BP1 | NP_665899.1 | RNA binding protein | Y358 | VyAADPYHHALAPAPTYGVGAMASIYR | SEQ ID NO: 239 |
| 241 | 28P1 | NP_665899.1 | RNA binding protein | Y363 | VYAADPyHHALAPAPTYGVGAMASIYR | SEQ ID NO: 240 |
| 242 | CASC3 | NP_031385.2 | RNA binding protein | Y313 | HQGLGGTLPPRTFINRNAAGTGRMSAP | SEQ ID NO: 241 |
| | | | | | RNySR | |
| 243 | CSTF2 | NP_001316.1 | RNA binding protein | Y115 | SLGTGAPVIESPyGETISPEDAPESISK | SEQ ID NO: 242 |
| 244 | CSTF3 | NP_001317.1 | RNA binding protein | Y71 | FWKLyIEAEIKAKNYDKVEK | SEQ ID NO: 243 |
| 245 | FXR1 | | RNA binding protein | Y477 | DPDSNPySLLDNTESDQTADTDASESHH | SEQ ID NO: 244 |
| | | | | | STNR | |
| 246 | GLE1L | NP_00100372 | RNA binding protein | Y547 | KCPYSVPFYPTFKEGMALEDyQRMLGY | SEQ ID NO: 245 |
| | | 2.1 | | | QVKDSK | |
| 247 | HNRPR | NP_005817.1 | RNA binding protein | Y434 | STAYEDyYYHPPPR | SEQ ID NO: 246 |
| 248 | ILF3 | NP_004507.2 | RNA binding protein | Y355 | PKNENPVDyTVQIPPSTTYAITPMKRPME | SEQ ID NO: 247 |
| | | | | | EDGEEK | |
| 249 | ILF3 | NP_004507.2 | RNA binding protein | Y365 | PKNENPVDYTVQIPPSTTyAITPMKRPME | SEQ ID NO: 248 |
| | | | | | EDGEEK | |
| 250 | PABPCS | NP_543022.1 | RNA binding protein | Y15 | yLKAALYVGDLDPDVTEDMLYKK | SEQ ID NO: 249 |
| 251 | RAE1 | NP_00101588 | RNA binding protein | Y274 | SNGTNTSAPQDIyAVNGIAFHPVHGTLAT | SEQ ID NO: 250 |
| | | 5.1 | | | VGSDGR | |
| 252 | RBM14 | NP_006319.1 | RNA binding protein | Y645 | LPDAHSDyARYSGSYNDYLR | SEQ ID NO: 251 |
| 253 | RBM14 | NP_006319.1 | RNA binding protein | Y648 | LPDAHSDYARySGSYNDYLR | SEQ ID NO: 252 |
| 254 | R8M14 | NP_006319.1 | RNA binding protein | Y655 | LPDAHSDYARYSGSYNDyLRAAQMHSG | SEQ ID NO: 253 |
| | | | | | QRRM | |
| 255 | RBM3 | NP_006734.1 | RNA binding protein | Y118 | YyDSRPGGYGYGYGRSR | SEQ ID NO: 254 |
| 256 | SNRPB2 | NP_003083.1 | RNA binding protein | Y28 | RSLyALFSQFGHVVDIVALKTMKMR | SEQ ID NO: 255 |
| 257 | SYNCRIP | NP_006363.3 | RNA binding protein | Y481 | GGyEDPYYGYEDFQVGARGRGGRGAR | SEQ ID NO: 256 |
| | | | | | GAAPSR | |
| 258 | C1QA | NP_057075.1 | Secreted protein | Y84 | GDQGEPGPSGNPGKVGyPGPSGPLGA | SEQ ID NO: 257 |
| | | | | | RGIPGIK | |
| 259 | CHGB | NP_001810.1 | Secreted protein | Y173 | SQREDEEEEEGENyQKGER | SEQ ID NO: 258 |
| 260 | CHGB | NP_001810.1 | Secreted protein | Y362 | GYPGVQAPEDLEWERyRGR | SEQ ID NO: 259 |
| 261 | F8 | NP_000123.1 | Secreted protein | Y2124 | FSSLYISQFIIMySLDGKKWQTYR | SEQ ID NO: 260 |
| 262 | F8 | NP_000123.1 | Secreted protein | Y2134 | FSSLYISQFIIMYSLDGKKWQTyR | SEQ ID NO: 261 |
| 263 | SEMG1 | NP_002998.1 | Secreted protein | Y220 | NSHQNKGHyQNVVEVREEHSSK | SEQ ID NO: 262 |
| 264 | SERP1 | NP_003003.3 | Secreted protein | Y127 | PIyPCRWLCEAVRDSCEPVMQFFGFYW | SEQ ID NO: 263 |
| | | | | | PEMLK | |
| 265 | WNT4 | NP110388.2 | Secreted protein | Y80 | NLEVMDSVRRGAQLAIEECQyQFR | SEQ ID NO: 264 |
| 266 | BARX1 | NP_067545.2 | Transcription factor | Y161 | LSTPDRIDLAESLGLSQLQVKTWyQN | SEQ ID NO: 265 |
| | | | | | RR | |
| 267 | CREB5 | NP878901.2 | Transcription factor | Y3 | MIyEESKMNLEQER | SEQ ID NO: 266 |
| 268 | DCP1A | NP_060873.3 | Transcription factor | Y64 | SASPyHGFTIVNR | SEQ ID NO: 267 |
| 269 | EGR1 | | Transcription factor | Y26 | EMQLMSPLQISDPFGSFPHsPTMDNY | SEQ ID NO: 268 |
| | | | | | PK | |
| 270 | GATA6 | NP_005248.2 | Transcription factor | Y310 | EPGGYAAAGSGGAGGVSGGGSSLAAM | SEQ ID NO: 269 |
| | | | | | GGREPQySSLSAAR | |
| 271 | GATA6 | NP_005248.2 | Transcription factor | Y409 | RDGTGHyLCNACGLYSKMNGLSR | SEQ ID NO: 270 |
| 272 | HIC1 | NP_006488.2 | Transcription factor | Y136 | HGKyCHLRGGGGGGGGYAPYGR | SEQ ID NO: 271 |
| 273 | HIC1 | NP_006488.2 | Transcription factor | Y149 | HGKYCHLRGGGGGGGGyAPYGR | SEQ ID NO: 272 |
| 274 | HIC1 | NP_006488.2 | Transcription factor | Y152 | HGKYCHLRGGGGGGGGYAPyGR | SEQ ID NO: 273 |
| 275 | LITAF | NP_004853.2 | Transcription factor | Y23 | TGPSSAPSAPPSyEET | SEQ ID NO: 274 |
| 276 | MECT1 | NP_056136.1 | Transcription factor | Y133 | RQADSCPyGTMYLSP | SEQ ID NO: 275 |
| 277 | MLL | NP_005924.2 | Transcription factor | Y2136 | PPHSQTSGSCYyHVISKVPRIRTPSYSPT | SEQ ID NO: 276 |
| | | | | | QR | |
| 278 | MLX | NP_733752.1 | Transcription factor | Y215 | KDVTALKIMKVNyEQIVK | SEQ ID NO: 277 |
| 279 | MYOD1 | NP_002469.2 | Transcription factor | Y230 | RNCYEGAyYNEAPSEPRPGK | SEQ ID NO: 278 |
| 280 | NFATC1 | NP_006153.2 | Transcription factor | Y688 | RKRSQyQRFTYLPANVPIIK | SEQ ID NO: 279 |
| 281 | PBX2 | NP_002577.2 | Transcription factor | Y384 | HSMGPGGyGDNLGGGQMYSPREMR | SEQ ID NO: 280 |
| 282 | PHOX2A | NP_005160.2 | Transcription factor | Y75 | DHQPAPYSAVPyKFFPEPSGLHEKR | SEQ ID NO: 281 |
| 283 | PITX2 | NP_000316.2 | Transcription factor | Y116 | QRTHFTSQQLQELEATFQRNRyPDMS | SEQ ID NO: 282 |
| | | | | | TR | |
| 284 | PRKCBP1 | NP_036540.3 | Transcription factor | Y369 | SIFNSAMQEMEVyVENIRRK | SEQ ID NO: 283 |
| 285 | R.AI1 | NP_109590.3 | Transcription factor | Y185 | THSLHVQQPPPPQQPLAyPK | SEQ ID NO: 284 |
| 286 | RFX4 | NP_002911.2 | Transcription factor | Y214 | LGTLLPEFPNVKDLNLPASLPEEKVSTFI | SEQ ID NO: 285 |
| | | | | | MMyR | |
| 287 | RUNX3 | NP_004341.1 | TranscripUon factor | Y280 | MHYPGAMSAAFPySATPSGTSISSLSVA | SEQ ID NO: 286 |
| | | | | | GMPATSR | |
| 288 | SOX7 | NP113627.1 | Transcription factor | Y109 | LQHMQDyPNYKYR | SEQ ID NO: 287 |
| 289 | SOX7 | NP113627.1 | Transcription factor | Y112 | LQHMQDYPNyKYR | SEQ ID NO: 288 |
| 290 | TBX1 | NP_005983.1 | Transcription factor | Y38 | MHFSTVTRDMEAFTASSLSSLGAAGGFP | SEQ ID NO: 289 |
| | | | | | GAASPGADPyGPR | |
| 291 | TBX5 | NP_000183.2 | Transcription factor | Y100 | VTGLNPKTKyILLMDIVPADDHRYK | SEQ ID NO: 290 |
| 292 | TBX5 | NP_000183.2 | Transcription factor | Y114 | VTGLNPKTKYILLMDIVPADDHRyK | SEQ ID NO: 291 |
| 293 | TCF12 | NP_003196.1 | Transcription factor | Y195 | KVPPGLPSSVyAPSPNSDDFNR | SEQ ID NO: 292 |
| 294 | ZNF267 | NP_003405.2 | Transcnption factor | Y615 | ECGKAFSySSDVIQHR | SEQ ID NO: 293 |
| 295 | GTF2E1 | NP_005504.1 | Transcription initiation | Y91 | HNyYFINYR | SEQ ID NO: 294 |
| | | | complex | | | |
| 296 | GTF2H1 | NP_005307.1 | Transcription initiation | Y516 | QyLSTNLVSHIEEMLQTAYNK | SEQ ID NO: 295 |
| | | | complex | | | |
| 297 | GTF2H1 | NP_005307.1 | Transcription initiation | Y533 | QYLSTNLVSHIEEMLQTAyNK | SEQ ID NO: 296 |
| | | | complex | | | |
| 298 | GTF3C5 | NP_036219.1 | Transcription initiation | Y305 | VLLPFIAYYMITGPWRSLWIRFGyDPR | SEQ ID NO: 297 |
| | | | complex | | | |
| 299 | POLR1B | NP_061887.2 | Transcription initiation | Y136 | GIIKQFLGyVPIMVKSK | SEQ ID NO: 298 |
| | | | complex | | | |
| 300 | POLR1B | NP_061887.2 | Transcription initiation | Y1118 | FVAELAAMNIK | SEQ ID NO: 299 |
| | | | complex | | | |
| 301 | POLR3C | NP_006459.3 | Transcription initiation | Y396 | QVEDFAMIPAKEAKDMLyKMLSENFMSL | SEQ ID NO: 300 |
| | | | complex | | QEIPK | |
| 302 | POLRMT | NP_005026.3 | Transcription initiation | Y386 | LLRDVYAKDGRVSyPK | SEQ ID NO: 301 |
| | | | complex | | | |
| 303 | PTRF | NP_036364.2 | Transcription initiation | Y156 | VMIyQDEVK | SEQ ID NO: 302 |
| | | | complex | | | |
| 304 | PTRF | | Transcription initiation | Y308 | KSFTPDHVVyAR | SEQ ID NO: 303 |
| | | | complex | | | |
| 305 | ES | NP_001121.2 | Transcription, | Y64 | HYVMyYEMSYGLNIEMHKQAEIVKR | SEQ ID NO: 304 |
| | | | coactivator/corepressor | | | |
| 306 | ES | NP_001121.2 | Transcription, | Y69 | HYVMYYEMSyGLNIEMHKQAEIVKR | SEQ ID NO: 305 |
| | | | coactivator/corepressor | | | |
| 307 | NKRD12 | NP_056023.2 | Transcription, | Y1229 | PPVEyDSDFMLESSESQMSFSQSPFLSI | SEQ ID NO: 306 |
| | | | coactivator/corepressor | | K | |
| 308 | BCOR | NP_060215.4 | Transcription | Y1527 | LLLSYGADPTLATySGRTIMK | SEQ ID NO: 307 |
| | | | coactivator/corepressor | | | |
| 309 | BRD8 | NP_006687.3 | Transcription | Y167 | LEEEEAEVKRKATDAAyQARQAVK | SEQ ID NO: 308 |
| | | | coactivator/corepressor | | | |
| 310 | CXXC1 | NP_055408.1 | Transcription, | Y509 | yESQTSFGSMYPTR | SEQ ID NO: 309 |
| | | | coactivator/corepressor | | | |
| 311 | CXXC1 | NP_055408.1 | Transcription, | Y519 | YESQTSFGSMyPTR | SEQ ID NO: 310 |
| | | | coactivator/corepressor | | | |
| 312 | EP400 | NP_056224.2 | Transcription, | Y1432 | LKASRLFQPVQyGQKPEGRTVAFPSTHP | SEQ ID NO: 311 |
| | | | coactivator/corepressor | | PR | |
| 313 | HSFY1 | NP149099.2 | Transcription, | Y175 | LKFyYNPNFK | SEQ ID NO: 312 |
| | | | coactivator/corepressor | | | |
| 314 | HSFY1 | NP149099.2 | Transcription, | Y176 | LKFYyNPNFK | SEQ ID NO: 313 |
| | | | coactivator/corepressor | | | |
| 315 | HSGT1 | NP_009196.1 | Transcription, | Y64 | KPGKGGVPAHMFGVTK | SEQ ID NO: 314 |
| | | | coactivator/corepressor | | | |
| 316 | JARID1A | NP_005047.2 | Transcrption, | Y148 | VGSRLGyLPGKGTGSLLK | SEQ ID NO: 315 |
| | | | coactivator/corepressor | | | |
| 317 | MKL2 | NP_054767.3 | Transcription | Y305 | yHQYIPPDQKGEKNEPQMDSNYAR | SEQ ID NO: 316 |
| | | | coactivator/corepressor | | | |
| 318 | MTA1 | NP_004680.1 | Transcription | Y659 | MNWIDAPGDVFyMPK | SEQ ID NO: 317 |
| | | | coactivator/corepressor | | | |
| 319 | PQBP1 | NP_005701.1 | Transcription, | Y187 | REELAPyPK | SEQ ID NO: 318 |
| | | | coactivator/corepressor | | | |
| 320 | PQBP1 | NP_005701.1 | Transcription, | Y209 | VSRKDEELDPMDPSSySDAPR | SEQ ID NO: 319 |
| | | | coactivator/corepressor | | | |
| 321 | PR1C285 | NP_208384.2 | Transcription | Y1845 | yHEDAHMLDTQYRMHEGICAFPSVAFYK | SEQ ID NO: 320 |
| | | | coactivator/corepressor | | SKLK | |
| 322 | PR10285 | NP_208384.2 | Transcription, | Y1871 | YHEDAHMLDTQYRMHEGICAFPSVAFyK | SEQ ID NO: 321 |
| | | | coactivator/corepressor | | SKLK | |
| 323 | TBL1XR1 | NP_078941.2 | Transcription | Y446 | HQEPVySVAFSPDGR | SEQ ID NO: 322 |
| | | | coactivator/corepressor | | | |
| 324 | THRAP3 | NP_005110.1 | Transcription | Y412 | PFRGSQSPKRyKLR | SEQ ID NO: 323 |
| | | | coactivator/corepressor | | | |
| 325 | TNIP1 | NP_006049.2 | Transcription | Y7 | GPyRIYDPGGSVPSGEASAAFER | SEQ ID NO: 324 |
| | | | coactivator/corepressor | | | |
| 326 | TNIP1 | NP_006049.2 | Transcription, | Y10 | GPYRIyDPGGSVPSGEASAAFER | SEQ ID NO: 325 |
| | | | coactivator/corepressor | | | |
| 327 | TP53BP2 | | Transcription, | Y541 | QQHPENIySNSQGKP | SEQ ID NO: 326 |
| | | | coactivator/corepressor | | | |
| 328 | YAP1 | NP_006097.1 | Transcription, | Y188 | yFLNHIDQTTTWQDPR | SEQ ID NO: 327 |
| | | | coactivator/corepressor | | | |
| 329 | ZBTB33 | NP_006768.1 | Transcription, | Y493 | HDDHYELIVDGRVyYICIVCKRSYVCLTS | SEQ ID NO: 328 |
| | | | coactivator/corepressor | | LR | |
| 330 | ZBTB33 | NP_006768.1 | Transcription, | Y503 | HDDHYELIVDGRVYYICIVCKRSyVCLTS | SEQ ID NO: 329 |
| | | | coactivator/corepressor | | LR | |
| 331 | B3GALT3 | NP_003772.1 | Transferase | Y175 | yVMKTDTDVFINTGNLVK | SEQ ID NO: 330 |
| 332 | CHST7 | NP_063939.2 | Transferase | Y414 | GAAyGADRPFHLSARDAREAVHAWR | SEQ ID NO: 331 |
| 333 | EXT1 | NP_000118.2 | Transferase | Y284 | NALyHVHNGEDVVLLTTCK | SEQ ID NO: 332 |
| 334 | F13A1 | | Transferase | Y482 | LIVTKQIGGDGMMDITDTyK | SEQ ID NO: 333 |
| 335 | GALGT | NP_001469.1 | Transferase | Y504 | yRYPGSLDESQMAKHR | SEQ ID NO: 334 |
| 336 | GALNT3 | NP_004473.1 | Transferase | Y1O1 | QNIDAGERPCLQGyYTAAELK | SEQ ID NO: 335 |
| 337 | GALNT3 | NP_004473.1 | Transferase | Y102 | QNIDAGERPCLQGYyTAAELK | SEQ ID NO: 336 |
| 338 | HRMT1L3 | NP_005779.1 | Transferase | Y387 | IAFWDDVyGFK | SEQ ID NO: 337 |
| 339 | MTR | NP_000245.1 | Transferase | Y701 | yPRPLNIIEGPLMNGMK | SEQ ID NO: 338 |
| 340 | MTR | NP_000245.1 | Transferase | Y988 | PFFDVWQLRGKyPNR | SEQ ID NO: 339 |
| 341 | NDST3 | NP_004775.1 | Transferase | Y489 | HTIFYKEyPGGPKEL | SEQ ID NO: 340 |
| 342 | POFUT1 | | Transferase | Y211 | yMVWSDEMVK | SEQ ID NO: 341 |
| 343 | POMT1 | NP_009102.2 | Transferase | Y581 | YSSSPLEWVTLDTNIAyWLHPR | SEQ ID NO: 342 |
| 344 | SOAT1 | NP_003092.4 | Transferase | Y312 | SSTVPIPTVNQYLYFLFAPTLIYRDSyPRN | SEQ ID NO: 343 |
| | | | | | PTVR | |
| 345 | ST8SIA1 | NP_003025.1 | Transferase | Y217 | TFVDNMKIYNHSyIYMPAFSMK | SEQ ID NO: 344 |
| 346 | SULT1C2 | NP_006579.2 | Transferase | Y200 | ILYLFyEDMKKNPK | SEQ ID NO: 345 |
| 347 | SULT4A1 | NP_055166.1 | Transferase | Y114 | SHLPyRFLPSDLHNGDSKVIYMARNPK | SEQ ID NO: 346 |
| 348 | SULT4A1 | NP_055166.1 | Transferase | Y130 | SHLPYRFLPSDLHNGDSKVIyMARNPK | SEQ ID NO: 347 |
| 349 | TPST1 | NP_003587.1 | Transferase | Y350 | VyKGEFQLPDFLKEKPQTEQVE | SEQ ID NO: 348 |
| 350 | UGT2B10 | NP_001066.1 | Transferase | Y192 | PPSyVPVVMSKLSDQMTFMERVKNML | SEQ ID NO: 349 |
| 351 | EEF1A2 | NP_001949.1 | Translation initiation | Y85 | FETTKyYITIIDAPGHR | SEQ ID NO: 350 |
| | | | complex | | | |
| 352 | EEF1E1 | NP_004271.1 | Translation initiation | Y107 | VyLTGYNFTLADILLYYGLHR | SEQ ID NO: 351 |
| | | | complex | | | |
| 353 | EEF1E1 | NP_004271.1 | Translation initiation | Y111 | VYLTGyNFTLADILLYYGLHR | SEQ ID NO: 352 |
| | | | complex | | | |
| 354 | EIF3S6IP | NP_057175.1 | Translation initiation | Y17 | SEAAYDPyAYPSDYD | SEQ ID NO: 353 |
| | | | complex | | | |
| 355 | EIF3S6IP | NP_057175.1 | Translation initiation | Y19 | AAYDPYAyPSDYDMH | SEQ ID NO: 354 |
| | | | complex | | | |
| 356 | EIF3S6IP | NP_057175.1 | Translation initiation | Y539 | DMIHIADTKVARRyGDFFIRQIHK | SEQ ID NO: 355 |
| | | | complex | | | |
| 357 | EIF3S8 | NP_003743.1 | Translation initiation | Y913 | QQQSQTAy | SEQ ID NO: 356 |
| | | | complex | | | |
| 358 | EIF3S9 | NP_003742.2 | Translation initiation | Y339 | ARWTETyVR | SEQ ID NO: 357 |
| | | | complex | | | |
| 359 | EIF4B | NP_001408.2 | Translation initiation | Y105 | LPKSPPYTAFLGNLPyDVTEESIK | SEQ ID NO: 358 |
| | | | complex | | | |
| 360 | RPL7A | NP_000963.1 | Translation initiation | Y226 | TNyNDRYDEIRRHWGGNVLGPKSVAR | SEQ ID NO: 359 |
| | | | complex | | | |
| 361 | RPL7A | NP_000963.1 | Translation initiation | Y230 | TNYNDRyDEIRRHWGGNVLGPKSVAR | SEQ ID NO: 360 |
| | | | complex | | | |
| 362 | RPS13 | | Translation initiation | Y38 | KLTSDDVKEQIyKL | SEQ ID NO: 361 |
| | | | complex | | | |
| 363 | RPS16 | NP_001011.1 | Translation initiation | Y82 | GGGHVAQIyAIR | SEQ ID NO: 362 |
| | | | complex | | | |
| 364 | RPS3 | NP_000996.2 | Translation initiation | Y120 | ACyGVLR | SEQ ID NO: 363 |
| | | | complex | | | |
| 365 | TAF15 | NP_003478.1 | Translation initiation | Y434 | GGRGGDRGGYGGDRSGGGYGGDRSS | SEQ ID NO: 364 |
| | | | complex; RNA binding | | GGGySGDR | |
| | | | protein | | | |
| 366 | TAF15 | NP_003478.1 | Translation initiation | Y443 | SSGGGYSGDRSGGGyGGDRSGGGYGG | SEQ ID NO: 365 |
| | | | complex; RNA binding | | DRGGGYGGDR | |
| | | | protein | | | |
| 367 | TAF15 | | Translation initiation | Y460 | GGGyGGDRGGYGGKMGGRNDYRND | SEQ ID NO: 366 |
| | | | complex; RNA binding | | QR | |
| | | | protein | | | |
| 368 | TAF15 | | Translation initiation | Y491 | GGGyGGDRGGYGGKMGGRNDYRND | SEQ ID NO: 367 |
| | | | complex; RNA binding | | QR | |
| | | | protein | | | |
| 369 | TAF15 | NP_003478.1 | Translation initiation | Y528 | GGGyGGDRGGYGGKMGGRNDYRND | SEQ ID NO: 368 |
| | | | complex; RNA binding | | QR | |
| | | | protein | | | |
| 370 | TAF15 | NP_003478.1 | Translation initiation | Y538 | GGYGGDRGGGSGyGGDR | SEQ ID NO: 369 |
| | | | complex; RNA binding | | | |
| | | | protein | | | |
| 371 | 6004 | NP_005836.1 | Transporter, ABC | Y617 | DGKMVQKGTyTEFLKSGIDFGSLLK | SEQ ID NO: 370 |
| 372 | BCD3 | NP_002849.1 | Transporter, active | Y261 | LRRPIGKMTITEQKyEGEYRYVNSR | SEQ ID NO: 371 |
| 373 | BCD3 | NP_002849.1 | Transporter, active | Y265 | LRRPIGKMTITEQKYEGEyR | SEQ ID NO: 372 |
| 374 | ATP1A1 | NP_000692.2 | Transporter, active | Y542 | EQPLDEELKDAFQNAyLELGGLGER | SEQ ID NO: 373 |
| 375 | Atp1a3 | NP_689509.1 | Transporter, active | Y548 | VLGFCHyYLPEEQFPK | SEQ ID NO: 374 |
| 376 | Atp1a3 | NP_689509.1 | Transporter, active | Y549 | VLGFCHYyLPEEQFPK | SEQ ID NO: 375 |
| 377 | ATP7B | NP_000044.2 | Transporter, active | Y187 | NQEAVITyQPYLIQP | SEQ ID NO: 376 |
| 378 | ATP8B2 | NP_065185.1 | Transporter, active | Y1162 | SGyAFSHQEGFGELIMSGKNMR | SEQ ID NO: 377 |
| 379 | CDW92 | NP_071392.2 | Transporter, active | Y263 | VLVWILTILVILGSLGGTGVLWWLyAK | SEQ ID NO: 378 |
| 380 | CDW92 | NP_071392.2 | Transporter, active | Y617 | YNDGSPGREFyMDKVLMEFVENSRKA | SEQ ID NO: 379 |
| | | | | | MK | |
| 381 | SLC7A11 | NP_055146.1 | Transporter, active | Y15 | GGyLQGNVNGR | SEQ ID NO: 380 |
| 382 | HBA2 | NP_000508.1 | Transporter, facilitator | Y25 | VGAHAGEyGAEALER | SEQ ID NO: 381 |
| 383 | Hba-a1 | NP_005328.2 | Transporter, facilitator | Y25 | IGGHGAEyGAEALER | SEQ ID NO: 382 |
| 384 | MATP | NP_00101252 | Transporter, facilitator | Y105 | PyILTLGVMMLVGMALYLNGATWAALIA | SEQ ID NO: 383 |
| | | 7.1 | | | NPR | |
| 385 | SLC12A2 | NP_001037.1 | Transporter, facilitator | Y227 | IDHyRHTAAQLGEK | SEQ ID NO: 384 |
| 386 | SLC12A2 | NP_001037.1 | Transporter, facilitator | Y275 | DAVVTyTAESK | SEQ ID NO: 385 |
| 387 | SLC27A2 | NP_003636.1 | Transporter, facilitator | Y304 | yNVTVIQYIGELLRYLCNSPQKPNDR | SEQ ID NO: 386 |
| 388 | SLC27A2 | NP_003636.1 | Transporter, facilitator | Y311 | YNVTVIQyIGELLRYLCNSPQKPNDR | SEQ ID NO: 387 |
| 389 | SLC38A2 | NP_061849.2 | Transporter, facilitator | Y20 | FSISPDEDSSSySSNSDFNYSYPTK | SEQ ID NO: 388 |
| 390 | SLC38A2 | NP_061849.2 | Transporter, facilitator | Y28 | FSISPDEDSSSYSSNSDFNySYPTK | SEQ ID NO: 389 |
| 391 | SLC39A6 | NP_036451.2 | Transporter, facilitator | Y522 | HAHPQEVyNEYVPRG | SEQ ID NO: 390 |
| 392 | SLC6A15 | NP_060527.2 | Transporter, facilitator | Y99 | NGGGAyLLPYLILLMVIGIPLFFLELSVGQ | SEQ ID NO: 391 |
| | | | | | RIR | |
| 393 | SLC6A15 | NP_060527.2 | Transporter, facilitator | Y103 | NGGGAYLLPyLILLMVIGIPLFFLELSVGQ | SEQ ID NO: 392 |
| | | | | | RIR | |
| 394 | SLC9A1 | NP_003038.2 | Transporter, facilitator | Y366 | PyVEANISHKSHTTIKYFLK | SEQ ID NO: 393 |
| 395 | SLC9A1 | NP_003038.2 | Transporter, facilitator | Y381 | PYVEANISHKSHTTIKyFLK | SEQ ID NO: 394 |
| 396 | PC | NP_000029.2 | Tumor suppressor | Y737 | NLMANRPAKyKDANIMSPGSSLPSLHV | SEQ ID NO: 395 |
| | | | | | RK | |
| 397 | LZTS1 | NP_066300.1 | Tumor suppressor | Y295 | LQRSFEEKELASSLAEERPR | SEQ ID NO: 396 |
| 398 | PHF3 | NP_055968.1 | Tumor suppressor | Y1291 | EICVVRFTPVTEEDQISYTLLFAyFSSRKR | SEQ ID NO: 397 |
| 399 | RB1 | NP_000312.2 | Tumor suppressor | Y239 | LSPPMLLKEPyKTAVIPINGSPR | SEQ ID NO: 398 |
| 400 | SLIT2 | NP_004778.1 | Tumor suppressor | Y1502 | RKySFECTDGSSFVDEVEKWK | SEQ ID NO: 399 |
| 401 | TES | NP_056456.1 | Tumor suppressor | Y111 | KNVSINTVTyEWAPPVQNQALAR | SEQ ID NO: 400 |
| 402 | TP53 | NP_000537.2 | Tumor suppressor; | Y327 | KKPLDGEyFTLQIR | SEQ ID NO: 401 |
| | | | Transcription factor; | | | |
| | | | Activator protein | | | |
| 403 | COPS6 | NP_006824.2 | Ubiquitin conjugating | Y105 | EYyYTKEEQFK | SEQ ID NO: 402 |
| | | | system | | | |
| 404 | COPS6 | NP_006824.2 | Ubiquitin conjugating | Y106 | EYYyTKEEQFK | SEQ ID NO: 403 |
| | | | system | | | |
| 405 | CUL2 | NP_003582.2 | Ubiquitin conjugating | Y43 | ATWNDRFSDIyALCVAYPEPLGER | SEQ ID NO: 404 |
| | | | system | | | |
| 406 | CUL5 | NP_003469.2 | Ubiquitin conjugating | Y214 | FyRTQAPSYLQQNGVQNYMK | SEQ ID NO: 405 |
| | | | system | | | |
| 407 | CUL5 | NP_003469.2 | Ubiquitin conjugating | Y221 | FYRTQAPSyLQQNGVQNYMK | SEQ ID NO: 406 |
| | | | system | | | |
| 408 | CUL5 | NP_003469.2 | Ubiquitin conjugating | Y230 | FYRTQAPSYLQQNGVQNyMK | SEQ ID NO: 407 |
| | | | system | | | |
| 409 | HERC4 | NP_071362.1 | Ubiquitin conjugating | Y895 | QEFVDAYVDyIFNKSVASLFDAFHAGFHK | SEQ ID NO: 408 |
| | | | system | | VCGGK | |
| 410 | MGRN1 | | Ubiquitin conjugating | Y411 | AIPSAPLyEEITYSG | SEQ ID NO: 409 |
| | | | system | | | |
| 411 | MGRN1 | | Ubiquitin conjugating | Y416 | PLYEEITySGISDGL | SEQ ID NO: 410 |
| | | | system | | | |
| 412 | NEDD4 | NP_006145.1 | Ubiquitin conjugating | Y43 | VIAGIGLAKKDILGASDPVR | SEQ ID NO: 411 |
| | | | system | | | |
| 413 | NEDD4 | NP_006145.1 | Ubiquitin conjugating | Y150 | VKGYLRLKMTyLPK | SEQ ID NO: 412 |
| | | | system | | | |
| 414 | NYREN18 | NP_057202.2 | Ubiquitin conjugating | Y126 | IAETFGLQENyIK | SEQ ID NO: 413 |
| | | | system | | | |
| 415 | TNFAIP3 | NP_006281.1 | Ubiquitin conjugating | Y111 | TNGDGNCLMHATSQyMWGVQDTDLVL | SEQ ID NO: 414 |
| | | | system | | RK | |
| 416 | TRIAD3 | NP_996994.1 | Ubiquitin conjugating | Y370 | NYyDLNVLCNFLLENPDYPK | SEQ ID NO: 415 |
| | | | system | | | |
| 417 | TRIAD3 | NP_996994.1 | Ubiquitin conjugating | Y385 | NYyDLNVLCNFLLENPDyPK | SEQ ID NO: 416 |
| | | | system | | | |
| 418 | UBE2E1 | NP_003332.1 | Ubiquitin conjugating | Y77 | ELADITLDPPPNCSAGPKGDNIyEWR | SEQ ID NO: 417 |
| | | | system | | | |
| 419 | UBE2J1 | NP_057105.2 | Ubiquitin conjugating | Y5 | yNLKSPAVKRLMK | SEQ ID NO: 418 |
| | | | system | | | |
| 420 | USP10 | NP_005144.1 | Ubiquitin conjugating | Y503 | DIRPGAAFEPTyIYRLLTVNKSSLSEK | SEQ ID NO: 419 |
| | | | system | | | |
| 421 | USP10 | NP_005144.1 | Ubiquitin conjugating | Y505 | DIRPGAAFEPTYIyRLLTVNKSSLSEK | SEQ ID NO: 420 |
| | | | system | | | |
| 422 | ZA20D1 | NP_064590.1 | Ubiquitin conjugating | Y794 | VADSYSNGyREPPEPDGWAGGLR | SEQ ID NO: 421 |
| | | | system | | | |
| 423 | AP1M1 | NP_115882.1 | Vesicle protein | Y354 | EyLMRAHFGLPSVEAEDK | SEQ ID NO: 422 |
| 424 | CLTC | NP_004850.1 | Vesicle protein | Y899 | FLRENPyYDSR | SEQ ID NO: 423 |
| 425 | DYSF | NP_003485.1 | Vesicle protein | Y1157 | CyMYQARDLAAMDKDSFSDPYAIVSFLH | SEQ ID NO: 424 |
| | | | | | QSQK | |
| 426 | DYSE | NP_003485.1 | Vesicle protein | Y1159 | CYMyQARDLAAMDKDSFSDPYAIVSFLH | SEQ ID NO: 425 |
| | | | | | QSQK | |
| 427 | DYSF | NP_003485.1 | Vesicle protein | Y1176 | CYMYQARDLAAMDKDSFSDPyAIVSFLH | SEQ ID NO: 426 |
| | | | | | QSQK | |
| 428 | ENTH | NP_055481.1 | Vesicle protein | Y21 | VRELVDKATNWMNySEIESK | SEQ ID NO: 427 |
| 429 | ENTH | NP_055481.1 | Vesicle protein | Y159 | NKDKyVGVSSDSVGGFR | SEQ ID NO: 428 |
| 430 | GOLGA3 | NP_005886.2 | Vesicle protein | Y210 | ASTLAMTKEySFLR | SEQ ID NO: 429 |
| 431 | GOLGA4 | NP_002069.2 | Vesicle protein | Y2148 | NVyATTVGTPYK | SEQ ID NO: 430 |
| 432 | GOLGB1 | NP_004478.1 | Vesicle protein | Y3005 | SSSSQTQPLKVQyQR | SEQ ID NO: 431 |
| 433 | GOLPH4 | NP_055313.1 | Vesicle protein | Y673 | GREEHyEEEEEEEEDGAAVAEK | SEQ ID NO: 432 |
| 434 | SCAMP3 | | Vesicle protein | Y35 | QyATLDVYNPFETR | SEQ ID NO: 433 |
| 435 | SCAMP4 | NP_524558.1 | Vesicle protein | Y205 | EAQyNNFSGNSLPEYPTVPSYPGSGQ | SEQ ID NO: 434 |
| | | | | | WP | |
| 436 | SEC10L1 | NP_006535.1 | Vesicle protein | Y356 | QTFLSKLIKSIFISYLENYIEVETGyLKSR | SEQ ID NO: 435 |
| 437 | SEC3L1 | NP_060731.2 | Vesicle protein | Y403 | YAKLMEWLKSTDYGKyEGLTK | SEQ ID NO: 436 |
| 438 | SEC3L1 | NP_060731.2 | Vesicle protein | Y800 | VAQGIREEEVSyQLAFNKQELR | SEQ ID NO: 437 |
| 439 | SEC8L1 | NP_068579.3 | Vesicle protein | Y247 | KFLDTSHySTAGSSSVR | SEQ ID NO: 438 |
| 440 | SNX25 | NP_114159.2 | Vesicle protein | Y151 | PVVELLSNPOyINQMLLAQLAYREQMNE | SEQ ID NO: 439 |
| | | | | | HHK | |
| 441 | SNX9 | NP_057308.1 | Vesicle protein | Y219 | ASSSSMKIPLNKFPGFAKPGTEQyLLAK | SEQ ID NO: 440 |
| 442 | STX4A | NP_004595.2 | Vesicle protein | Y251 | NILSSADyVER | SEQ ID NO: 441 |
| 443 | TSG101 | NP_006283.1 | Vesicle protein | Y390 | KTAGLSDLy | SEQ ID NO: 442 |
| 444 | VPS28 | NP_057292.1 | Vesicle protein | Y36 | EKyDNMAELFAVVKTMQALEK | SEQ ID NO: 443 |

The short name for each protein in which a phosphorylation site has presently been identified is provided in Column A, and its SwissProt accession number (human) is provided Column B. The protein type/group into which each protein falls is provided in Column C. The identified tyrosine residue at which phosphorylation occurs in a given protein is identified in Column D, and the amino acid sequence of the phosphorylation site encompassing the tyrosine residue is provided in Column E (lower case y=the tyrosine (identified in Column D)) at which phosphorylation occurs. Table 1 above is identical to FIG. 2, except that the latter includes the disease and cell type(s) in which the particular phosphorylation site was identified (Columns F and G).

The identification of these 443 phosphorylation sites is described in more detail in Part A below and in Example 1.

### DEFINITIONS

As used herein, the following terms have the meanings indicated:

“Antibody” or “antibodies” refers to all types of immunoglobulins, including IgG, IgM, IgA, IgD, and IgE, including F_{ab} or antigen-recognition fragments thereof, including chimeric, polyclonal, and monoclonal antibodies. The term “does not bind” with respect to an antibody's binding to one phospho-form of a sequence means does not substantially react with as compared to the antibody's binding to the other phospho-form of the sequence for which the antibody is specific.

“Carcinoma-related signaling protein” means any protein (or poly-peptide derived therefrom) enumerated in Column A of Table 1/FIG. 2, which is disclosed herein as being phosphorylated in one or more human carcinoma cell line(s). Carcinoma-related signaling proteins may be protein kinases, or direct substrates of such kinases, or may be indirect substrates downstream of such kinases in signaling pathways. A Carcinoma-related signaling protein may also be phosphorylated in other cell lines (non-carcinomic) harboring activated kinase activity.

“Heavy-isotope labeled peptide” (used interchangeably with AQUA peptide) means a peptide comprising at least one heavy-isotope label, which is suitable for absolute quantification or detection of a protein as described in WO/03016861, “Absolute Quantification of Proteins and Modified Forms Thereof by Multistage Mass Spectrometry” (Gygi et al.), further discussed below.

“Protein” is used interchangeably with polypeptide, and includes protein fragments and domains as well as whole protein.

“Phosphorylatable amino acid” means any amino acid that is capable of being modified by addition of a phosphate group, and includes both forms of such amino acid.

“Phosphorylatable peptide sequence” means a peptide sequence comprising a phosphorylatable amino acid.

“Phosphorylation site-specific antibody” means an antibody that specifically binds a phosphorylatable peptide sequence/epitope only when phosphorylated, or only when not phosphorylated, respectively. The term is used interchangeably with “phospho-specific” antibody.

### A. Identification of Novel Carcinoma-Related Signaling Protein Phosphorylation Sites.

The nearly 443 novel Carcinoma-related signaling protein phosphorylation sites disclosed herein and listed in Table 1/FIG. 2 were discovered by employing the modified peptide isolation and characterization techniques described in “Immunoaffinity Isolation of Modified Peptides From Complex Mixtures,” U.S. Patent Publication No. 20030044848, Rush et al. (the teaching of which is hereby incorporated herein by reference, in its entirety) using cellular extracts from the human carcinoma derived cell lines and patient samples indicated in Column G of Table 1/FIG. 2. Exemplary cell lines used include sw480, 293T, 293T TNT-TAT Silac, 293TTS ATIC-ALK, CTV-1, JB, Karpas 299, MOLT15, MV4-11, SU-DHL1, H196, H1993, Calu-3, HCT116, A431, U118 MG, DMS 153, SCLC T1, MDA-MB-468 and H1703. The isolation and identification of phosphopeptides from these cell lines, using an immobilized general phosphotyrosine-specific antibody, is described in detail in Example 1 below. In addition to the nearly 443 previously unknown protein phosphorylation sites (tyrosine) discovered, many known phosphorylation sites were also identified (not described herein).

The immunoaffinity/mass spectrometric technique described in the '848 patent Publication (the “IAP” method)—and employed as described in detail in the Examples—is briefly summarized below.

The IAP method employed generally comprises the following steps: (a) a proteinaceous preparation (e.g. a digested cell extract) comprising phosphopeptides from two or more different proteins is obtained from an organism; (b) the preparation is contacted with at least one immobilized general phosphotyrosine-specific antibody; (c) at least one phosphopeptide specifically bound by the immobilized antibody in step (b) is isolated; and (d) the modified peptide isolated in step (c) is characterized by mass spectrometry (MS) and/or tandem mass spectrometry (MS-MS). Subsequently, (e) a search program (e.g. Sequest) may be utilized to substantially match the spectra obtained for the isolated, modified peptide during the characterization of step (d) with the spectra for a known peptide sequence. A quantification step employing, e.g. SILAC or AQUA, may also be employed to quantify isolated peptides in order to compare peptide levels in a sample to a baseline.

In the IAP method as employed herein, a general phosphotyrosine-specific monoclonal antibody (commercially available from Cell Signaling Technology, Inc., Beverly, Mass., Cat #9411 (p-Tyr-100)) was used in the immunoaffinity step to isolate the widest possible number of phospho-tyrosine containing peptides from the cell extracts. Extracts from the human carcinoma cell lines described above were employed.

As described in more detail in the Examples, lysates were prepared from these cells line and digested with trypsin after treatment with DTT and iodoacetamide to alkylate cysteine residues. Before the immunoaffinity step, peptides were pre-fractionated by reversed-phase solid phase extraction using Sep-Pak C₁₈ columns to separate peptides from other cellular components. The solid phase extraction cartridges were eluted with varying steps of acetonitrile. Each lyophilized peptide fraction was redissolved in IAP buffer and treated with phosphotyrosine-specific antibody (P-Tyr-100, CST #9411) immobilized on protein Agarose. Immunoaffinity-purified peptides were eluted with 0.1% TFA and a portion of this fraction was concentrated with Stage or Zip tips and analyzed by LC-MS/MS, using a ThermoFinnigan LCQ Deca XP Plus ion trap mass spectrometer. Peptides were eluted from a 10 cm×75 μm reversed-phase column with a 45-min linear gradient of acetonitrile. MS/MS spectra were evaluated using the program Sequest with the NCBI human protein database.

This revealed a total of nearly 443 novel tyrosine phosphorylation sites in signaling pathways affected by kinase activation or active in carcinoma cells. The identified phosphorylation sites and their parent proteins are enumerated in Table 1/FIG. 2. The tyrosine (human sequence) at which phosphorylation occurs is provided in Column D, and the peptide sequence encompassing the phosphorylatable tyrosine residue at the site is provided in Column E. FIG. 2 also shows the particular type of carcinoma (see Column G) and cell line(s) (see Column F) in which a particular phosphorylation site was discovered.

As a result of the discovery of these phosphorylation sites, phospho-specific antibodies and AQUA peptides for the detection of and quantification of these sites and their parent proteins may now be produced by standard methods, described below. These new reagents will prove highly useful in, e.g., studying the signaling pathways and events underlying the progression of carcinomas and the identification of new biomarkers and targets for diagnosis and treatment of such diseases.

### B. Antibodies and Cell Lines

Isolated phosphorylation site-specific antibodies that specifically bind a Carcinoma-related signaling protein disclosed in Column A of Table 1 only when phosphorylated (or only when not phosphorylated) at the corresponding amino acid and phosphorylation site listed in Columns D and E of Table 1/FIG. 2 may now be produced by standard antibody production methods, such as anti-peptide antibody methods, using the phosphorylation site sequence information provided in Column E of Table 1. For example, previously unknown Ser/Thr kinase phosphorylation site (tyrosine 351) (see Row 146 of Table 1/FIG. 2) is presently disclosed. Thus, antibodies that specifically bind this novel Ser/Thr kinase site can now be produced, e.g. by immunizing an animal with a peptide antigen comprising all or part of the amino acid sequence encompassing the respective phosphorylated residue (e.g. a peptide antigen comprising the sequence set forth in Rows 146 of Column E, of Table 1 (SEQ ID NO: 145) (which encompasses the phosphorylated tyrosine at positions 351 of the Ser/Thr kinase), to produce an antibody that only binds Ser/Thr kinase when phosphorylated at that site.

Polyclonal antibodies of the invention may be produced according to standard techniques by immunizing a suitable animal (e.g., rabbit, goat, etc.) with a peptide antigen corresponding to the Carcinoma-related phosphorylation site of interest (i.e. a phosphorylation site enumerated in Column E of Table 1, which comprises the corresponding phosphorylatable amino acid listed in Column D of Table 1), collecting immune serum from the animal, and separating the polyclonal antibodies from the immune serum, in accordance with known procedures. For example, a peptide antigen corresponding to all or part of the novel Receptor tyrosine kinase phosphorylation site disclosed herein (SEQ ID NO: 19=DDGMEEVVGHTQGPLDGSLyAK, encompassing phosphorylated tyrosine 365 (lowercase y; see Row 20 of Table 1)) may be used to produce antibodies that only bind Receptor tyrosine kinase phosphorylation when phosphorylated at tyr365. Similarly, a peptide comprising all or part of any one of the phosphorylation site sequences provided in Column E of Table 1 may employed as an antigen to produce an antibody that only binds the corresponding protein listed in Column A of Table 1 when phosphorylated (or when not phosphorylated) at the corresponding residue listed in Column D. If an antibody that only binds the protein when phosphorylated at the disclosed site is desired, the peptide antigen includes the phosphorylated form of the amino acid. Conversely, if an antibody that only binds the protein when not phosphorylated at the disclosed site is desired, the peptide antigen includes the non-phosphorylated form of the amino acid.

Peptide antigens suitable for producing antibodies of the invention may be designed, constructed and employed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)).

It will be appreciated by those of skill in the art that longer or shorter phosphopeptide antigens may be employed. See Id. For example, a peptide antigen may comprise the full sequence disclosed in Column E of Table 1/FIG. 2, or it may comprise additional amino acids flanking such disclosed sequence, or may comprise of only a portion of the disclosed sequence immediately flanking the phosphorylatable amino acid (indicated in Column E by lowercase “y”). Typically, a desirable peptide antigen will comprise four or more amino acids flanking each side of the phosphorylatable amino acid and encompassing it. Polyclonal antibodies produced as described herein may be screened as further described below.

Monoclonal antibodies of the invention may be produced in a hybridoma cell line according to the well-known technique of Kohler and Milstein. See Nature 265:495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, C. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246:1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

The preferred epitope of a phosphorylation-site specific antibody of the invention is a peptide fragment consisting essentially of about 8 to 17 amino acids including the phosphorylatable tyrosine, wherein about 3 to 8 amino acids are positioned on each side of the phosphorylatable tyrosine (for example, the OCLN tyrosine 315 phosphorylation site sequence disclosed in Row 44, Column E of Table 1), and antibodies of the invention thus specifically bind a target Carcinoma-related signaling polypeptide comprising such epitopic sequence. Particularly preferred epitopes bound by the antibodies of the invention comprise all or part of a phosphorylatable site sequence listed in Column E of Table 1, including the phosphorylatable amino acid.

Included in the scope of the invention are equivalent non-antibody molecules, such as protein binding domains or nucleic acid aptamers, which bind, in a phospho-specific manner, to essentially the same phosphorylatable epitope to which the phospho-specific antibodies of the invention bind. See, e.g., Neuberger et al., Nature 312: 604 (1984). Such equivalent non-antibody reagents may be suitably employed in the methods of the invention further described below.

Antibodies provided by the invention may be any type of immunoglobulins, including IgG, IgM, IgA, IgD, and IgE, including F_{ab} or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,443,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.)

The invention also provides immortalized cell lines that produce an antibody of the invention. For example, hybridoma clones, constructed as described above, that produce monoclonal antibodies to the Carcinoma-related signaling protein phosphorylation sties disclosed herein are also provided. Similarly, the invention includes recombinant cells producing an antibody of the invention, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Phosphorylation site-specific antibodies of the invention, whether polyclonal or monoclonal, may be screened for epitope and phospho-specificity according to standard techniques. See, e.g. Czernik et al., Methods in Enzymology, 201: 264-283 (1991). For example, the antibodies may be screened against the phospho and non-phospho peptide library by ELISA to ensure specificity for both the desired antigen (i.e. that epitope including a phosphorylation site sequence enumerated in Column E of Table 1) and for reactivity only with the phosphorylated (or non-phosphorylated) form of the antigen. Peptide competition assays may be carried out to confirm lack of reactivity with other phospho-epitopes on the given Carcinoma-related signaling protein. The antibodies may also be tested by Western blotting against cell preparations containing the signaling protein, e.g. cell lines over-expressing the target protein, to confirm reactivity with the desired phosphorylated epitope/target.

Specificity against the desired phosphorylated epitope may also be examined by constructing mutants lacking phosphorylatable residues at positions outside the desired epitope that are known to be phosphorylated, or by mutating the desired phospho-epitope and confirming lack of reactivity. Phosphorylation-site specific antibodies of the invention may exhibit some limited cross-reactivity to related epitopes in non-target proteins. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-target proteins is readily characterized by Western blotting alongside markers of known molecular weight. Amino acid sequences of cross-reacting proteins may be examined to identify sites highly homologous to the Carcinoma-related signaling protein epitope for which the antibody of the invention is specific.

In certain cases, polyclonal antisera may exhibit some undesirable general cross-reactivity to phosphotyrosine itself, which may be removed by further purification of antisera, e.g. over a phosphotyramine column. Antibodies of the invention specifically bind their target protein (i.e. a protein listed in Column A of Table 1) only when phosphorylated (or only when not phosphorylated, as the case may be) at the site disclosed in corresponding Columns D/E, and do not (substantially) bind to the other form (as compared to the form for which the antibody is specific).

Antibodies may be further characterized via immunohistochemical (IHC) staining using normal and diseased tissues to examine Carcinoma-related phosphorylation and activation status in diseased tissue. IHC may be carried out according to well-known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies may be further characterized by flow cytometry carried out according to standard methods. See Chow et al., Cytometry (Communications in Clinical Cytometry) 46: 72-78 (2001). Briefly and by way of example, the following protocol for cytometric analysis may be employed: samples may be centrifuged on Ficoll gradients to remove erythrocytes, and cells may then be fixed with 2% paraformaldehyde for 10 minutes at 37° C. followed by permeabilization in 90% methanol for 30 minutes on ice. Cells may then be stained with the primary phosphorylation-site specific antibody of the invention (which detects a Carcinoma-related signal transduction protein enumerated in Table 1), washed and labeled with a fluorescent-labeled secondary antibody. Additional fluorochrome-conjugated marker antibodies (e.g. CD45, CD34) may also be added at this time to aid in the subsequent identification of specific hematopoietic cell types. The cells would then be analyzed on a flow cytometer (e.g. a Beckman Coulter FC500) according to the specific protocols of the instrument used.

Antibodies of the invention may also be advantageously conjugated to fluorescent dyes (e.g. Alexa488, PE) for use in multi-parametric analyses along with other signal transduction (phospho-CrkL, phospho-Erk 1/2) and/or cell marker (CD34) antibodies.

Phosphorylation-site specific antibodies of the invention specifically bind to a human Carcinoma-related signal transduction protein or polypeptide only when phosphorylated at a disclosed site, but are not limited only to binding the human species, per se. The invention includes antibodies that also bind conserved and highly homologous or identical phosphorylation sites in respective Carcinoma-related proteins from other species (e.g. mouse, rat, monkey, yeast), in addition to binding the human phosphorylation site. Highly homologous or identical sites conserved in other species can readily be identified by standard sequence comparisons, such as using BLAST, with the human Carcinoma-related signal transduction protein phosphorylation sites disclosed herein.

### C. Heavy-Isotope Labeled Peptides (AQUA Peptides).

The novel Carcinoma-related signaling protein phosphorylation sites disclosed herein now enable the production of corresponding heavy-isotope labeled peptides for the absolute quantification of such signaling proteins (both phosphorylated and not phosphorylated at a disclosed site) in biological samples. The production and use of AQUA peptides for the absolute quantification of proteins (AQUA) in complex mixtures has been described. See WO/03016861, “Absolute Quantification of Proteins and Modified Forms Thereof by Multistage Mass Spectrometry,” Gygi et al., and also Gerber et al. Proc. Natl. Acad. Sci. U.S.A. 100: 6940-5 (2003) (the teachings of which are hereby incorporated herein by reference, in their entirety).

The AQUA methodology employs the introduction of a known quantity of at least one heavy-isotope labeled peptide standard (which has a unique signature detectable by LC-SRM chromatography) into a digested biological sample in order to determine, by comparison to the peptide standard, the absolute quantity of a peptide with the same sequence and protein modification in the biological sample. Briefly, the AQUA methodology has two stages: peptide internal standard selection and validation and method development; and implementation using validated peptide internal standards to detect and quantify a target protein in sample. The method is a powerful technique for detecting and quantifying a given peptide/protein within a complex biological mixture, such as a cell lysate, and may be employed, e.g., to quantify change in protein phosphorylation as a result of drug treatment, or to quantify differences in the level of a protein in different biological states.

Generally, to develop a suitable internal standard, a particular peptide (or modified peptide) within a target protein sequence is chosen based on its amino acid sequence and the particular protease to be used to digest. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a mass shift. A newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or modified protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al. supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g. trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures. Because an absolute amount of the AQUA peptide is added (e.g. 250 fmol), the ratio of the areas under the curve can be used to determine the precise expression levels of a protein or phosphorylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard is developed for a known phosphorylation site sequence previously identified by the IAP-LC-MS/MS method within a target protein. One AQUA peptide incorporating the phosphorylated form of the particular residue within the site may be developed, and a second AQUA peptide incorporating the non-phosphorylated form of the residue developed. In this way, the two standards may be used to detect and quantify both the phosphorylated and non-phosphorylated forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g. trypsin, hepsin), metallo proteases (e.g. PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. Thus, peptides longer than about 20 amino acids are not preferred. The preferred ranged is about 7 to 15 amino acids. A peptide sequence is also selected that is not likely to be chemically reactive during mass spectrometry, thus sequences comprising cysteine, tryptophan, or methionine are avoided.

A peptide sequence that does not include a modified region of the target region may be selected so that the peptide internal standard can be used to determine the quantity of all forms of the protein. Alternatively, a peptide internal standard encompassing a modified amino acid may be desirable to detect and quantify only the modified form of the target protein. Peptide standards for both modified and unmodified regions can be used together, to determine the extent of a modification in a particular sample (i.e. to determine what fraction of the total amount of protein is represented by the modified form). For example, peptide standards for both the phosphorylated and unphosphorylated form of a protein known to be phosphorylated at a particular site can be used to quantify the amount of phosphorylated form in a sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: The mass should be unique to shift fragment masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the 20 natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ¹³C, ¹⁵N, ¹⁷O, ¹⁸O, or ³⁴S, are among preferred labels. Pairs of peptide internal standards that incorporate a different isotope label may also be prepared. Preferred amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts are preferably employed. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g. by HPLC, reverse-phase HPLC, capillary electrophoresis, ion exchange chromatography, etc.) to isolate the labeled internal standard and its corresponding target peptide from other peptides in the sample. Microcapillary LC is a preferred method.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

In accordance with the present invention, AQUA internal peptide standards (heavy-isotope labeled peptides) may now be produced, as described above, for any of the nearly 443 novel Carcinoma-related signaling protein phosphorylation sites disclosed herein (see Table 1/FIG. 2). Peptide standards for a given phosphorylation site (e.g. the tyrosine 136 site in HIC1—see Row 272 of Table 1) may be produced for both the phosphorylated and non-phosphorylated forms of the site (e.g. see HIC1 site sequence in Column E, Row 272 of Table 1 (SEQ ID NO: 271)) and such standards employed in the AQUA methodology to detect and quantify both forms of such phosphorylation site in a biological sample.

AQUA peptides of the invention may comprise all, or part of, a phosphorylation site peptide sequence disclosed herein (see Column E of Table 1/FIG. 2). In a preferred embodiment, an AQUA peptide of the invention consists of, or comprises, a phosphorylation site sequence disclosed herein in Table 1/FIG. 2. For example, an AQUA peptide of the invention for detection/quantification of PIK3CB kinase when phosphorylated at tyrosine 436 may consist of, or comprise, the sequence TINPSKYQTIRKAGKVHyPVAWVNTMVFDFK (y=phosphotyrosine), which comprises phosphorylatable tyrosine 436 (see Row 139, Column E; (SEQ ID NO: 138)). Heavy-isotope labeled equivalents of the peptides enumerated in Table 1/FIG. 2 (both in phosphorylated and unphosphorylated form) can be readily synthesized and their unique MS and LC-SRM signature determined, so that the peptides are validated as AQUA peptides and ready for use in quantification experiments.

The phosphorylation site peptide sequences disclosed herein (see Column E of Table 1/FIG. 2) are particularly well suited for development of corresponding AQUA peptides, since the IAP method by which they were identified (see Part A above and Example 1) inherently confirmed that such peptides are in fact produced by enzymatic digestion (trypsinization) and are in fact suitably fractionated/ionized in MS/MS. Thus, heavy-isotope labeled equivalents of these peptides (both in phosphorylated and unphosphorylated form) can be readily synthesized and their unique MS and LC-SRM signature determined, so that the peptides are validated as AQUA peptides and ready for use in quantification experiments.

Accordingly, the invention provides heavy-isotope labeled peptides (AQUA peptides) for the detection and/or quantification of any of the Carcinoma-related phosphorylation sites disclosed in Table 1/FIG. 2 (see Column E) and/or their corresponding parent proteins/polypeptides (see Column A). A phosphopeptide sequence consisting of, or comprising, any of the phosphorylation sequences listed in Table 1 may be considered a preferred AQUA peptide of the invention. For example, an AQUA peptide comprising the sequence TQTVRGTLAYLPEEyIKTGR (SEQ ID NO: 146) (where y may be either phosphotyrosine or tyrosine, and where V=labeled valine (e.g. ¹⁴C)) is provided for the quantification of phosphorylated (or non-phosphorylated) kinase (Tyr 395) in a biological sample (see Row 147 of Table 1, tyrosine 395 being the phosphorylatable residue within the site). However, it will be appreciated that a larger AQUA peptide comprising a disclosed phosphorylation site sequence (and additional residues downstream or upstream of it) may also be constructed. Similarly, a smaller AQUA peptide comprising less than all of the residues of a disclosed phosphorylation site sequence (but still comprising the phosphorylatable residue enumerated in Column D of Table 1/FIG. 2) may alternatively be constructed. Such larger or shorter AQUA peptides are within the scope of the present invention, and the selection and production of preferred AQUA peptides may be carried out as described above (see Gygi et al., Gerber et al. supra.).

Certain particularly preferred subsets of AQUA peptides provided by the invention are described above (corresponding to particular protein types/groups in Table 1, for example, Kinases or Adaptor/Scaffold proteins). Example 4 is provided to further illustrate the construction and use, by standard methods described above, of exemplary AQUA peptides provided by the invention. For example, the above-described AQUA peptides corresponding to the both the phosphorylated and non-phosphorylated forms of the disclosed PTPN11 phosphatase tyrosine 263 phosphorylation site (see Row 195 of Table 1/FIG. 2) may be used to quantify the amount of phosphorylated PTPN11 phosphatase (Tyr 263) in a biological sample, e.g. a tumor cell sample (or a sample before or after treatment with a test drug).

AQUA peptides of the invention may also be employed within a kit that comprises one or multiple AQUA peptide(s) provided herein (for the quantification of a Carcinoma-related signal transduction protein disclosed in Table 1/FIG. 2), and, optionally, a second detecting reagent conjugated to a detectable group. For example, a kit may include AQUA peptides for both the phosphorylated and non-phosphorylated form of a phosphorylation site disclosed herein. The reagents may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

AQUA peptides provided by the invention will be highly useful in the further study of signal transduction anomalies underlying cancer, including carcinomas, and in identifying diagnostic/bio-markers of these diseases, new potential drug targets, and/or in monitoring the effects of test compounds on Carcinoma-related signal transduction proteins and pathways.

### D. Immunoassay Formats

Antibodies provided by the invention may be advantageously employed in a variety of standard immunological assays (the use of AQUA peptides provided by the invention is described separately above). Assays may be homogeneous assays or heterogeneous assays. In a homogeneous assay the immunological reaction usually involves a phosphorylation-site specific antibody of the invention), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, a phosphorylation-site specific antibody of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof that may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of antigen-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of a target Carcinoma-related signal transduction protein is detectable compared to background.

Phosphorylation site-specific antibodies disclosed herein may be conjugated to a solid support suitable for a diagnostic assay (e.g., beads, plates, slides or wells formed from materials such as latex or polystyrene) in accordance with known techniques, such as precipitation. Antibodies, or other target protein or target site-binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Antibodies of the invention may also be optimized for use in a flow cytometry (FC) assay to determine the activation/phosphorylation status of a target Carcinoma-related signal transduction protein in patients before, during, and after treatment with a drug targeted at inhibiting phosphorylation at such a protein at the phosphorylation site disclosed herein. For example, bone marrow cells or peripheral blood cells from patients may be analyzed by flow cytometry for target Carcinoma-related signal transduction protein phosphorylation, as well as for markers identifying various hematopoietic cell types. In this manner, activation status of the malignant cells may be specifically characterized. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 1% para-formaldehyde for 10 minutes at 37° C. followed by permeabilization in 90% methanol for 30 minutes on ice. Cells may then be stained with the primary antibody (a phospho-specific antibody of the invention), washed and labeled with a fluorescent-labeled secondary antibody. Alternatively, the cells may be stained with a fluorescent-labeled primary antibody. The cells would then be analyzed on a flow cytometer (e.g. a Beckman Coulter EPICS-XL) according to the specific protocols of the instrument used. Such an analysis would identify the presence of activated Carcinoma-related signal transduction protein(s) in the malignant cells and reveal the drug response on the targeted protein.

Alternatively, antibodies of the invention may be employed in immunohistochemical (IHC) staining to detect differences in signal transduction or protein activity using normal and diseased tissues. IHC may be carried out according to well-known techniques. See, e.g., ANTIBODIES: A LABORATORY MANUAL, supra. Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies of the invention may be also be optimized for use in other clinically-suitable applications, for example bead-based multiplex-type assays, such as IGEN, Luminex™ and/or Bioplex™ assay formats, or otherwise optimized for antibody arrays formats, such as reversed-phase array applications (see, e.g. Paweletz et al., Oncogene 20(16): 1981-89 (2001)). Accordingly, in another embodiment, the invention provides a method for the multiplex detection of Carcinoma-related protein phosphorylation in a biological sample, the method comprising utilizing two or more antibodies or AQUA peptides of the invention to detect the presence of two or more phosphorylated Carcinoma-related signaling proteins enumerated in Column A of Table 1/FIG. 2. In one preferred embodiment, two to five antibodies or AQUA peptides of the invention are employed in the method. In another preferred embodiment, six to ten antibodies or AQUA peptides of the invention are employed, while in another preferred embodiment eleven to twenty such reagents are employed.

Antibodies and/or AQUA peptides of the invention may also be employed within a kit that comprises at least one phosphorylation site-specific antibody or AQUA peptide of the invention (which binds to or detects a Carcinoma-related signal transduction protein disclosed in Table 1/FIG. 2), and, optionally, a second antibody conjugated to a detectable group. In some embodies, the kit is suitable for multiplex assays and comprises two or more antibodies or AQUA peptides of the invention, and in some embodiments, comprises two to five, six to ten, or eleven to twenty reagents of the invention. The kit may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

The following Examples are provided only to further illustrate the invention, and are not intended to limit its scope, except as provided in the claims appended hereto. The present invention encompasses modifications and variations of the methods taught herein which would be obvious to one of ordinary skill in the art.

### EXAMPLE 1

### Isolation of Phosphotyrosine-Containing Peptides from Extracts of Carcinoma Cell Lines and Identification of Novel Phosphorylation Sites

In order to discover previously unknown Carcinoma-related signal transduction protein phosphorylation sites, IAP isolation techniques were employed to identify phosphotyrosine-containing peptides in cell extracts from human carcinoma cell lines and patient cell lines identified in Column G of Table 1 including sw480, 293T, 293T TNT-TAT Silac, 293TTS ATIC-ALK, CTV-1, JB, Karpas 299, MOLT15, MV4-11, SU-DHL1, H196, H1993, Calu-3, HCT116, A431, U118 MG, DMS 153, SCLC T1, MDA-MB-468 and H1703. Tryptic phosphotyrosine-containing peptides were purified and analyzed from extracts of each of the cell lines mentioned above, as follows. Cells were cultured in DMEM medium or RPMI 1640 medium supplemented with 10% fetal bovine serum and penicillin/streptomycin.

Suspension cells were harvested by low speed centrifugation. After complete aspiration of medium, cells were resuspended in 1 mL lysis buffer per 1.25×10⁸ cells (20 mM HEPES pH 8.0, 9 M urea, 1 mM sodium vanadate, supplemented or not with 2.5 mM sodium pyrophosphate, 1 mM β-glycerol-phosphate) and sonicated.

Adherent cells at about 80% confluency were starved in medium without serum overnight and stimulated, with ligand depending on the cell type or not stimulated. After complete aspiration of medium from the plates, cells were scraped off the plate in 10 ml lysis buffer per 2×10⁸ cells (20 mM HEPES pH 8.0, 9 M urea, 1 mM sodium vanadate, supplemented with 2.5 mM sodium pyrophosphate, 1 mM β-glycerol-phosphate) and sonicated.

Sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and soluble TLCK-trypsin (Worthington) was added at 10-20 μg/mL. Digestion was performed for 1-2 days at room temperature.

Trifluoroacetic acid (TFA) was added to protein digests to a final concentration of 1%, precipitate was removed by centrifugation, and digests were loaded onto Sep-Pak C₁₈ columns (Waters) equilibrated with 0.1% TFA. A column volume of 0.7-1.0 ml was used per 2×10⁸ cells. Columns were washed with 15 volumes of 0.1% TFA, followed by 4 volumes of 5% acetonitrile (MeCN) in 0.1% TFA. Peptide fraction I was obtained by eluting columns with 2 volumes each of 8, 12, and 15% MeCN in 0.1% TFA and combining the eluates. Fractions II and III were a combination of eluates after eluting columns with 18, 22, 25% MeCN in 0.1% TFA and with 30, 35, 40% MeCN in 0.1% TFA, respectively. All peptide fractions were lyophilized.

Peptides from each fraction corresponding to 2×10⁸ cells were dissolved in 1 ml of IAP buffer (20 mM Tris/HCl or 50 mM MOPS pH 7.2, 10 mM sodium phosphate, 50 mM NaCl) and insoluble matter (mainly in peptide fractions III) was removed by centrifugation. IAP was performed on each peptide fraction separately. The phosphotyrosine monoclonal antibody P-Tyr-100 (Cell Signaling Technology, Inc., catalog number 9411) was coupled at 4 mg/ml beads to protein G (Roche), respectively. Immobilized antibody (15 μl, 60 μg) was added as 1:1 slurry in IAP buffer to 1 ml of each peptide fraction, and the mixture was incubated overnight at 4° C. with gentle rotation. The immobilized antibody beads were washed three times with 1 ml IAP buffer and twice with 1 ml water, all at 4° C. Peptides were eluted from beads by incubation with 75 μl of 0.1% TFA at room temperature for 10 minutes.

Alternatively, one single peptide fraction was obtained from Sep-Pak C18 columns by elution with 2 volumes each of 10%, 15%, 20%, 25%, 30%, 35% and 40% acetonitrile in 0.1% TFA and combination of all eluates. IAP on this peptide fraction was performed as follows: After lyophilization, peptide was dissolved in 50 ml IAP buffer (MOPS pH 7.2, 10 mM sodium phosphate, 50 mM NaCl) and insoluble matter was removed by centrifugation. Immobilized antibody (40 μl, 160 μg) was added as 1:1 slurry in IAP buffer, and the mixture was incubated overnight at 4° C. with gentle shaking. The immobilized antibody beads were washed three times with 1 ml IAP buffer and twice with 1 ml water, all at 4° C. Peptides were eluted from beads by incubation with 55 μl of 0.15% TFA at room temperature for 10 min (eluate 1), followed by a wash of the beads (eluate 2) with 45 μl of 0.15% TFA. Both eluates were combined.

### Analysis by LC-MS/MS Mass Spectrometry.

40 μl or more of IAP eluate were purified by 0.2 μl StageTips or ZipTips. Peptides were eluted from the microcolumns with 1 μl of 40% MeCN, 0.1% TFA (fractions I and II) or 1 μl of 60% MeCN, 0.1% TFA (fraction III) into 7.6-9.0 μl of 0.4% acetic acid/0.005% heptafluorobutyric acid. For single fraction analysis, 1 μl of 60% MeCN, 0.1% TFA, was used for elution from the microcolumns. This sample was loaded onto a 10 cm×75 μm PicoFrit capillary column (New Objective) packed with Magic C18 AQ reversed-phase resin (Michrom Bioresources) using a Famos autosampler with an inert sample injection valve (Dionex). The column was then developed with a 45-min linear gradient of acetonitrile delivered at 200 nl/min (Ultimate, Dionex), and tandem mass spectra were collected in a data-dependent manner with an LTQ ion trap mass spectrometer essentially as described by Gygi et al., supra.

### Database Analysis & Assignments.

MS/MS spectra were evaluated using TurboSequest in the Sequest Browser package (v. 27, rev. 12) supplied as part of BioWorks 3.0 (ThermoFinnigan). Individual MS/MS spectra were extracted from the raw data file using the Sequest Browser program CreateDta, with the following settings: bottom MW, 700; top MW, 4,500; minimum number of ions, 20; minimum TIC, 4×10⁵; and precursor charge state, unspecified. Spectra were extracted from the beginning of the raw data file before sample injection to the end of the eluting gradient. The IonQuest and VuDta programs were not used to further select MS/MS spectra for Sequest analysis. MS/MS spectra were evaluated with the following TurboSequest parameters: peptide mass tolerance, 2.5; fragment ion tolerance, 0.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were performed against the NCBI human protein database (NCBI RefSeq protein release #11; 8 May 2005; 1,826,611 proteins, including 47,859 human proteins. Peptides that did not match RefSeq were compared to NCBI GenPept release #148; 15 Jun. 2005 release date; 2,479,172 proteins, including 196,054 human proteins.). Cysteine carboxamidomethylation was specified as a static modification, and phosphorylation was allowed as a variable modification on serine, threonine, and tyrosine residues or on tyrosine residues alone. It was determined that restricting phosphorylation to tyrosine residues had little effect on the number of phosphorylation sites assigned.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell Proteomics 3: 531-533 (2004)), which were followed in this Example. However, because the immunoaffinity strategy separates phosphorylated peptides from unphosphorylated peptides, observing just one phosphopeptide from a protein is a common result, since many phosphorylated proteins have only one tyrosine-phosphorylated site. For this reason, it is appropriate to use additional criteria to validate phosphopeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic phosphopeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. The following Sequest scoring thresholds were used to select phosphopeptide assignments that are likely to be correct: RSp<6, XCorr≧2.2, and DeltaCN>0.099. Further, the sequence assignments could be accepted or rejected with respect to accuracy by using the following conservative, two-step process.

In the first step, a subset of high-scoring sequence assignments should be selected by filtering for XCorr values of at least 1.5 for a charge state of +1, 2.2 for +2, and 3.3 for +3, allowing a maximum RSp value of 10. Assignments in this subset should be rejected if any of the following criteria are satisfied: (i) the spectrum contains at least one major peak (at least 10% as intense as the most intense ion in the spectrum) that can not be mapped to the assigned sequence as an a, b, or y ion, as an ion arising from neutral-loss of water or ammonia from a b or y ion, or as a multiply protonated ion; (ii) the spectrum does not contain a series of b or y ions equivalent to at least six uninterrupted residues; or (iii) the sequence is not observed at least five times in all the studies conducted (except for overlapping sequences due to incomplete proteolysis or use of proteases other than trypsin).

In the second step, assignments with below-threshold scores should be accepted if the low-scoring spectrum shows a high degree of similarity to a high-scoring spectrum collected in another study, which simulates a true reference library-searching strategy.

### EXAMPLE 2

### Production of Phospho-Specific Polyclonal Antibodies for the Detection of Carcinoma-Related Signaling Protein Phosphorylation

Polyclonal antibodies that specifically bind a Carcinoma-related signal transduction protein only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, as further described below. Production of exemplary polyclonal antibodies is provided below.

### A. IRAK1 (Tyrosine 395).

A 20 amino acid phospho-peptide antigen, TQTVRGTLAYLPEEy*IKTGR (where y*=phosphotyrosine) that corresponds to the sequence encompassing the tyrosine 395 phosphorylation site in human IRAK kinase (see Row 147 of Table 1; SEQ ID NO: 146), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific IRAK1 (tyr 395) polyclonal antibodies as described in Immunization/Screening below.

### B. TNS1 (Tyrosine 366).

A 20 amino acid phospho-peptide antigen, TQTVRGTLAYLPEEy*IKTGR (where y*=phosphotyrosine) that corresponds to the sequence encompassing the tyrosine 366 phosphorylation site in human SPRY1 (see Row 20 of Table 1 (SEQ ID NO: 19)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific TNS1 (tyr 366) polyclonal antibodies as described in Immunization/Screening below.

### C. TBX1 (Tyrosine 38).

A 41 amino acid phospho-peptide antigen, MHFSTVTRDMEAFTASSLSSLGAAGGFPGAASPGADPy*GPR (where y*=phosphotyrosine) that corresponds to the sequence encompassing the tyrosine 38 phosphorylation site in human INPP5D protein (see Row 290 of Table 1 (SEQ ID NO: 289), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific TBX1 (tyr 38) antibodies as described in Immunization/Screening below.

### Immunization/Screening.

A synthetic phospho-peptide antigen as described in A-C above is coupled to KLH, and rabbits are injected intradermally (ID) on the back with antigen in complete Freunds adjuvant (500 μg antigen per rabbit). The rabbits are boosted with same antigen in incomplete Freund adjuvant (250 μg antigen per rabbit) every three weeks. After the fifth boost, bleeds are collected. The sera are purified by Protein A-affinity chromatography by standard methods (see ANTIBODIES: A LABORATORY MANUAL, Cold Spring Harbor, supra.). The eluted immunoglobulins are further loaded onto a non-phosphorylated synthetic peptide antigen-resin Knotes column to pull out antibodies that bind the non-phosphorylated form of the phosphorylation site. The flow through fraction is collected and applied onto a phospho-synthetic peptide antigen-resin column to isolate antibodies that bind the phosphorylated form of the site. After washing the column extensively, the bound antibodies (i.e. antibodies that bind a phosphorylated peptide described in A-C above, but do not bind the non-phosphorylated form of the peptide) are eluted and kept in antibody storage buffer.

The isolated antibody is then tested for phospho-specificity using Western blot assay using an appropriate cell line that expresses (or overexpresses) target phospho-protein (i.e. phosphorylated IRAK1, TNS1 or TBX1), for example, DU145 or DMS79. Cells are cultured in DMEM or RPMI supplemented with 10% FCS. Cell are collected, washed with PBS and directly lysed in cell lysis buffer. The protein concentration of cell lysates is then measured. The loading buffer is added into cell lysate and the mixture is boiled at 100° C. for 5 minutes. 20 μl (10 μg protein) of sample is then added onto 7.5% SDS-PAGE gel.

A standard Western blot may be performed according to the Immunoblotting Protocol set out in the CELL SIGNALING TECHNOLOGY, INC. 2003-04 Catalogue, p. 390. The isolated phospho-specific antibody is used at dilution 1:1000. Phosphorylation-site specificity of the antibody will be shown by binding of only the phosphorylated form of the target protein. Isolated phospho-specific polyclonal antibody does not (substantially) recognize the target protein when not phosphorylated at the appropriate phosphorylation site in the non-stimulated cells (e.g. TBX1 is not bound when not phosphorylated at tyrosine 38).

In order to confirm the specificity of the isolated antibody, different cell lysates containing various phosphorylated signal transduction proteins other than the target protein are prepared. The Western blot assay is performed again using these cell lysates. The phospho-specific polyclonal antibody isolated as described above is used (1:1000 dilution) to test reactivity with the different phosphorylated non-target proteins on Western blot membrane. The phospho-specific antibody does not significantly cross-react with other phosphorylated signal transduction proteins, although occasionally slight binding with a highly homologous phosphorylation-site on another protein may be observed. In such case the antibody may be further purified using affinity chromatography, or the specific immunoreactivity cloned by rabbit hybridoma technology.

### EXAMPLE 3

### Production of Phospho-Specific Monoclonal Antibodies for the Detection of Carcinoma-Related Signaling Protein Phosphorylation

Monoclonal antibodies that specifically bind a Carcinoma-related signal transduction protein only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, and harvesting spleen cells from such animals to produce fusion hybridomas, as further described below. Production of exemplary monoclonal antibodies is provided below.

### A. ILK (Tyrosine 351).

An 14 amino acid phospho-peptide antigen, My*APAWVAPEALQK (where y*=phosphotyrosine) that corresponds to the sequence encompassing the tyrosine 351 phosphorylation site in human ILK phosphatase (see Row 146 of Table 1 (SEQ ID NO: 145)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal ILK (tyr 351) antibodies as described in Immunization/Fusion/Screening below.

### B. TP53BP2 (Tyrosine 541).

A 15 amino acid phospho-peptide antigen, QQHPENIy*SNSQGKP (where y*=phosphotyrosine) that corresponds to the sequence encompassing the tyrosine 4505 phosphorylation site in human TP53BP2 (see Row 327 of Table 1 (SEQ ID NO: 326)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal TP53BP2 (tyr 541) antibodies as described in Immunization/Fusion/Screening below.

### C. APC (Tyrosine 737).

A 29 amino acid phospho-peptide antigen, NLMANRPAKy*KDANIMSPGSSLPSLHVRK (where y*=phosphotyrosines) that corresponds to the sequence encompassing the tyrosine 737 phosphorylation site in human APC protein (see Row 396 of Table 1 (SEQ ID NO: 395)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal APC (tyr 737) antibodies as described in Immunization/Fusion/Screening below.

### Immunization/Fusion/Screening.

A synthetic phospho-peptide antigen as described in A-C above is coupled to KLH, and BALB/C mice are injected intradermally (ID) on the back with antigen in complete Freunds adjuvant (e.g. 50 μg antigen per mouse). The mice are boosted with same antigen in incomplete Freund adjuvant (e.g. 25 μg antigen per mouse) every three weeks. After the fifth boost, the animals are sacrificed and spleens are harvested.

Harvested spleen cells are fused to SP2/0 mouse myeloma fusion partner cells according to the standard protocol of Kohler and Milstein (1975). Colonies originating from the fusion are screened by ELISA for reactivity to the phospho-peptide and non-phospho-peptide forms of the antigen and by Western blot analysis (as described in Example 1 above). Colonies found to be positive by ELISA to the phospho-peptide while negative to the non-phospho-peptide are further characterized by Western blot analysis. Colonies found to be positive by Western blot analysis are subcloned by limited dilution. Mouse ascites are produced from a single clone obtained from subcloning, and tested for phospho-specificity (against the ILK, TP53BP2, or APC) phospho-peptide antigen, as the case may be) on ELISA. Clones identified as positive on Western blot analysis using cell culture supernatant as having phospho-specificity, as indicated by a strong band in the induced lane and a weak band in the uninduced lane of the blot, are isolated and subcloned as clones producing monoclonal antibodies with the desired specificity.

Ascites fluid from isolated clones may be further tested by Western blot analysis. The ascites fluid should produce similar results on Western blot analysis as observed previously with the cell culture supernatant, indicating phospho-specificity against the phosphorylated target (e.g. ILK phosphorylated at tyrosine 351).

### EXAMPLE 4

### Production and Use of AQUA Peptides for the Quantification of Carcinoma-Related Signaling Protein Phosphorylation

Heavy-isotope labeled peptides (AQUA peptides (internal standards)) for the detection and quantification of a Carcinoma-related signal transduction protein only when phosphorylated at the respective phosphorylation site disclosed herein (see Table 1/FIG. 2) are produced according to the standard AQUA methodology (see Gygi et al., Gerber et al., supra.) methods by first constructing a synthetic peptide standard corresponding to the phosphorylation site sequence and incorporating a heavy-isotope label. Subsequently, the MSⁿ and LC-SRM signature of the peptide standard is validated, and the AQUA peptide is used to quantify native peptide in a biological sample, such as a digested cell extract. Production and use of exemplary AQUA peptides is provided below.

### A. NF1 (Tyrosine 2556).

An AQUA peptide comprising the sequence, RVAETDy*EMETQR (y*=phosphotyrosine; sequence incorporating ¹⁴C/¹⁵N-labeled valine (indicated by bold V), which corresponds to the tyrosine 2556 phosphorylation site in human PIK3C2B kinase (see Row 128 in Table 1 (SEQ ID NO: 127)), is constructed according to standard synthesis techniques using, e.g., a Rainin/Protein Technologies, Inc., Symphony peptide synthesizer (see Merrifield, supra.) as further described below in Synthesis & MS/MS Signature. The Met (tyr 835) AQUA peptide is then spiked into a biological sample to quantify the amount of phosphorylated NF1 (tyr 2556) in the sample, as further described below in Analysis & Quantification.

### B. TBX5 (Tyrosine 114).

An AQUA peptide comprising the sequence VTGLNPKTKYILLMDIVPADDHRy*K (y*=phosphotyrosine; sequence incorporating ¹⁴C/¹⁵N-labeled proline (indicated by bold P), which corresponds to the tyrosine 114 phosphorylation site in human TBX5 protein (see Row 292 in Table 1 (SEQ ID NO: 291)), is constructed according to standard synthesis techniques using, e.g., a Rainin/Protein Technologies, Inc., Symphony peptide synthesizer (see Merrifield, supra.) as further described below in Synthesis & MS/MS Signature. The TBX5 (tyr 114) AQUA peptide is then spiked into a biological sample to quantify the amount of phosphorylated TBX5 (tyr 114) in the sample, as further described below in Analysis & Quantification.

### C. RB1 (Tyrosine 239).

An AQUA peptide comprising the sequence LSPPMLLKEPy*KTAVIPINGSPR (y*=phosphotyrosine; sequence incorporating ¹⁴C/¹⁵N-labeled Leucine (indicated by bold L), which corresponds to the tyrosine 38 phosphorylation site in human VIM protein (see Row 399 in Table 1 (SEQ ID NO: 398)), is constructed according to standard synthesis techniques using, e.g., a Rainin/Protein Technologies, Inc., Symphony peptide synthesizer (see Merrifield, supra.) as further described below in Synthesis & MS/MS Signature. The RB1 (tyr 239) AQUA peptide is then spiked into a biological sample to quantify the amount of phosphorylated RB1 (tyr 239) in the sample, as further described below in Analysis & Quantification.

### D. MGRN1 (Tyrosine 416).

An AQUA peptide comprising the sequence PLYEEITySGISDGL (y*=phosphotyrosine; sequence incorporating ¹⁴C/¹⁵N-labeled proline (indicated by bold P), which corresponds to the tyrosine 416 phosphorylation site in human MGRN1 protein (see Row 411 in Table 1 (SEQ ID NO: 410)), is constructed according to standard synthesis techniques using, e.g., a Rainin/Protein Technologies, Inc., Symphony peptide synthesizer (see Merrifield, supra.) as further described below in Synthesis & MS/MS Signature. The MGRN1 (tyr 416) AQUA peptide is then spiked into a biological sample to quantify the amount of phosphorylated MGRN1 (tyr 416) in the sample, as further described below in Analysis & Quantification.

### Synthesis & MS/MS Spectra.

Fluorenylmethoxycarbonyl (Fmoc)-derivatized amino acid monomers may be obtained from AnaSpec (San Jose, Calif.). Fmoc-derivatized stable-isotope monomers containing one ¹⁵N and five to nine ¹³C atoms may be obtained from Cambridge Isotope Laboratories (Andover, Mass.). Preloaded Wang resins may be obtained from Applied Biosystems. Synthesis scales may vary from 5 to 25 μmol. Amino acids are activated in situ with 1-H-benzotriazolium, 1-bis(dimethylamino)methylene]-hexafluorophosphate (1-),3-oxide:1-hydroxybenzotriazole hydrate and coupled at a 5-fold molar excess over peptide. Each coupling cycle is followed by capping with acetic anhydride to avoid accumulation of one-residue deletion peptide by-products. After synthesis peptide-resins are treated with a standard scavenger-containing trifluoroacetic acid (TFA)-water cleavage solution, and the peptides are precipitated by addition to cold ether. Peptides (i.e. a desired AQUA peptide described in A-D above) are purified by reversed-phase C18 HPLC using standard TFA/acetonitrile gradients and characterized by matrix-assisted laser desorption ionization-time of flight (Biflex III, Bruker Daltonics, Billerica, Mass.) and ion-trap (ThermoFinnigan, LCQ DecaXP) MS.

MS/MS spectra for each AQUA peptide should exhibit a strong y-type ion peak as the most intense fragment ion that is suitable for use in an SRM monitoring/analysis. Reverse-phase microcapillary columns (0.1 Å˜150-220 mm) are prepared according to standard methods. An Agilent 1100 liquid chromatograph may be used to develop and deliver a solvent gradient [0.4% acetic acid/0.005% heptafluorobutyric acid (HFBA)/7% methanol and 0.4% acetic acid/0.005% HFBA/65% methanol/35% acetonitrile] to the microcapillary column by means of a flow splitter. Samples are then directly loaded onto the microcapillary column by using a FAMOS inert capillary autosampler (LC Packings, San Francisco) after the flow split. Peptides are reconstituted in 6% acetic acid/0.01% TFA before injection.

### Analysis & Quantification.

Target protein (e.g. a phosphorylated protein of A-D above) in a biological sample is quantified using a validated AQUA peptide (as described above). The IAP method is then applied to the complex mixture of peptides derived from proteolytic cleavage of crude cell extracts to which the AQUA peptides have been spiked in.

LC-SRM of the entire sample is then carried out. MS/MS may be performed by using a ThermoFinnigan (San Jose, Calif.) mass spectrometer (LCQ DecaXP ion trap or TSQ Quantum triple quadrupole). On the DecaXP, parent ions are isolated at 1.6 m/z width, the ion injection time being limited to 150 ms per microscan, with two microscans per peptide averaged, and with an AGC setting of 1×10⁸; on the Quantum, Q1 is kept at 0.4 and Q3 at 0.8 m/z with a scan time of 200 ms per peptide. On both instruments, analyte and internal standard are analyzed in alternation within a previously known reverse-phase retention window; well-resolved pairs of internal standard and analyte are analyzed in separate retention segments to improve duty cycle. Data are processed by integrating the appropriate peaks in an extracted ion chromatogram (60.15 m/z from the fragment monitored) for the native and internal standard, followed by calculation of the ratio of peak areas multiplied by the absolute amount of internal standard (e.g., 500 fmol).

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. An isolated phosphorylation site-specific antibody that specifically binds a human Carcinoma-related signaling protein selected from Column A of Table 1 only when phosphorylated at the tyrosine listed in corresponding Column D of Table 1, comprised within the phosphorylatable peptide sequence listed in corresponding Column E of Table 1 (SEQ ID NOs: 1-443), wherein said antibody does not bind said signaling protein when not phosphorylated at said tyrosine.

17. An isolated phosphorylation site-specific antibody that specifically binds a human Carcinoma-related signaling protein selected from Column A of Table 1 only when not phosphorylated at the tyrosine listed in corresponding Column D of Table 1, comprised within the phosphorylatable peptide sequence listed in corresponding Column E of Table 1 (SEQ ID NOs: 1-443), wherein said antibody does not bind said signaling protein when phosphorylated at said tyrosine.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. The heavy-isotope labeled peptide (AQUA peptide) of claim 18, wherein said labeled peptide is for the quantification of an apoptosis protein selected from Column A, Rows 58-60, said labeled peptide comprising the phosphorylatable peptide sequence listed in corresponding Column E, Rows 58-60, of Table 1 (SEQ ID NOs: 57-59), which sequence comprises the phosphorylatable tyrosine listed in corresponding Column D, Rows 58-60 of Table 1.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. An isolated phosphorylation site-specific antibody according to claim 16, that specifically binds a human Leukemia-related signaling protein selected from Column A, Rows 442, 382, 34, 202, 424, 223, 161 and 43 of Table 1 only when phosphorylated at the tyrosine listed in corresponding Column D of Table 1, comprised within the phosphorylatable peptide sequence listed in corresponding Column E of Table 1 (SEQ ID NOs: 441, 381, 33, 201, 423, 222, 160 and 42), wherein said antibody does not bind said signaling protein when not phosphorylated at said tyrosine.

54. An isolated phosphorylation site-specific antibody according to claim 17, that specifically binds a human Leukemia-related signaling protein selected from Column A, Rows 442, 382, 34, 202, 424, 223, 161 and 43 of Table 1 only when not phosphorylated at the tyrosine listed in corresponding Column D of Table 1, comprised within the phosphorylatable peptide sequence listed in corresponding Column E of Table 1 (SEQ ID NOs: SEQ ID NOs: 441, 381, 33, 201, 423, 222, 160 and 42), wherein said antibody does not bind said signaling protein when phosphorylated at said tyrosine.

55. A method selected from the group consisting of:
(a) a method for detecting a human leukemia-related signaling protein selected from Column A of Table 1, wherein said human leukemia-related signaling protein is phosphorylated at the tyrosine listed in corresponding Column D of Table 1, comprised within the phosphorylatable peptide sequence listed in corresponding Column E of Table 1 (SEQ ID NOs: 1-443), comprising the step of adding an isolated phosphorylation-specific antibody according to claim 16, to a sample comprising said human leukemia-related signaling protein under conditions that permit the binding of said antibody to said human leukemia-related signaling protein, and detecting bound antibody;
(b) a method for quantifying the amount of a human leukemia-related signaling protein listed in Column A of Table 1 that is phosphorylated at the corresponding tyrosine listed in Column D of Table 1, comprised within the phosphorylatable peptide sequence listed in corresponding Column E of Table 1 (SEQ ID NOs: 1-443), in a sample using a heavy-isotope labeled peptide (AQUA™ peptide), said labeled peptide comprising a phosphorylated tyrosine at said corresponding tyrosine listed Column D of Table 1, comprised within the phosphorylatable peptide sequence listed in corresponding Column E of Table 1 as an internal standard; and
(c) a method comprising step (a) followed by step (b).

56. The method of claim 55, wherein said isolated phosphorylation-specific antibody is capable of specifically binding STX4 only when phosphorylated at Y251, comprised within the phosphorylatable peptide sequence listed in Column E, Row 442, of Table 1 (SEQ ID NO: 442), wherein said antibody does not bind said protein when not phosphorylated at said tyrosine.

57. The method of claim 55, wherein said isolated phosphorylation-specific antibody is capable of specifically binding STX4 only when not phosphorylated at Y251, comprised within the phosphorylatable peptide sequence listed in Column E, Row 442, of Table 1 (SEQ ID NO: 441), wherein said antibody does not bind said protein when phosphorylated at said tyrosine.

58. The method of claim 55, wherein said isolated phosphorylation-specific antibody is capable of specifically binding HBA1 only when phosphorylated at Y25, comprised within the phosphorylatable peptide sequence listed in Column E, Row 382, of Table 1 (SEQ ID NO: 381), wherein said antibody does not bind said protein when not phosphorylated at said tyrosine.

59. The method of claim 55, wherein said isolated phosphorylation-specific antibody is capable of specifically binding HBA1 only when not phosphorylated at Y25, comprised within the phosphorylatable peptide sequence listed in Column E, Row 382, of Table 1 (SEQ ID NO: 381), wherein said antibody does not bind said protein when phosphorylated at said tyrosine.

60. The method of claim 55, wherein said isolated phosphorylation-specific antibody is capable of specifically binding F11R only when phosphorylated at Y280, comprised within the phosphorylatable peptide sequence listed in Column E, Row 34, of Table 1 (SEQ ID NO: 33), wherein said antibody does not bind said protein when not phosphorylated at said tyrosine.

61. The method of claim 55, wherein said isolated phosphorylation-specific antibody is capable of specifically binding F11R only when not phosphorylated at Y280, comprised within the phosphorylatable peptide sequence listed in Column E, Row 34, of Table 1 (SEQ ID NO: 33), wherein said antibody does not bind said protein when phosphorylated at said tyrosine.

62. The method of claim 55, wherein said isolated phosphorylation-specific antibody is capable of specifically binding PLCG1 only when phosphorylated at Y977, comprised within the phosphorylatable peptide sequence listed in Column E, Row 202, of Table 1 (SEQ ID NO: 201), wherein said antibody does not bind said protein when not phosphorylated at said tyrosine.

63. The method of claim 55, wherein said isolated phosphorylation-specific antibody is capable of specifically binding PLCG1 only when not phosphorylated at Y977, comprised within the phosphorylatable peptide sequence listed in Column E, Row 202, of Table 1 (SEQ ID NO: 201), wherein said antibody does not bind said protein when phosphorylated at said tyrosine.

64. The method of claim 55, wherein said isolated phosphorylation-specific antibody is capable of specifically binding CLTC only when phosphorylated at Y899, comprised within the phosphorylatable peptide sequence listed in Column E, Row 424, of Table 1 (SEQ ID NO: 423), wherein said antibody does not bind said protein when not phosphorylated at said tyrosine.

65. The method of claim 55, wherein said isolated phosphorylation-specific antibody is capable of specifically binding CLTC only when not phosphorylated at Y899, comprised within the phosphorylatable peptide sequence listed in Column E, Row 424, of Table 1 (SEQ ID NO: 423), wherein said antibody does not bind said protein when phosphorylated at said tyrosine.

66. The method of claim 55, wherein said isolated phosphorylation-specific antibody is capable of specifically binding NRP1 only when phosphorylated at Y920, comprised within the phosphorylatable peptide sequence listed in Column E, Row 223, of Table 1 (SEQ ID NO: 222), wherein said antibody does not bind said protein when not phosphorylated at said tyrosine.

67. The method of claim 55, wherein said isolated phosphorylation-specific antibody is capable of specifically binding NRP1 only when not phosphorylated at Y920, comprised within the phosphorylatable peptide sequence listed in Column E, Row 223, of Table 1 (SEQ ID NO: 222), wherein said antibody does not bind said protein when phosphorylated at said tyrosine.

68. The method of claim 55, wherein said isolated phosphorylation-specific antibody is capable of specifically binding EphA1 only when phosphorylated at Y781, comprised within the phosphorylatable peptide sequence listed in Column E, Row 1611, of Table 1 (SEQ ID NO: 160), wherein said antibody does not bind said protein when not phosphorylated at said tyrosine.

69. The method of claim 55, wherein said isolated phosphorylation-specific antibody is capable of specifically binding EphA1 only when not phosphorylated at Y781, comprised within the phosphorylatable peptide sequence listed in Column E, Row 161, of Table 1 (SEQ ID NO: 160), wherein said antibody does not bind said protein when phosphorylated at said tyrosine.

70. The method of claim 55, wherein said isolated phosphorylation-specific antibody is capable of specifically binding OCLN only when phosphorylated at Y287, comprised within the phosphorylatable peptide sequence listed in Column E, Row 43, of Table 1 (SEQ ID NO: 42), wherein said antibody does not bind said protein when not phosphorylated at said tyrosine.

71. The method of claim 55, wherein said isolated phosphorylation-specific antibody is capable of specifically binding OCLN only when not phosphorylated at Y287, comprised within the phosphorylatable peptide sequence listed in Column E, Row 43, of Table 1 (SEQ ID NO: 42), wherein said antibody does not bind said protein when phosphorylated at said tyrosine.


SEQUENCE LISTING (443 sequences)

SEQ ID NO: 1 - protein, 18 aa, Homo sapiens
  MOD_RES (1)..(1): Phosphorylated Tyr
YLAPVGPAGTLKAGRNTR

SEQ ID NO: 2 - protein, 13 aa, Homo sapiens
  MOD_RES (8)..(8): Phosphorylated Tyr
GPLDGSPYAQVQR

SEQ ID NO: 3 - protein, 18 aa, Homo sapiens
  MOD_RES (11)..(11): Phosphorylated Tyr
AGEEGHEGCSYTMCPEGR

SEQ ID NO: 4 - protein, 17 aa, Homo sapiens
  MOD_RES (15)..(15): Phosphorylated Tyr
LAFATHGTAFDKRPYHR

SEQ ID NO: 5 - protein, 17 aa, Homo sapiens
  MOD_RES (13)..(13): Phosphorylated Tyr
LSLDLSHRTCSDYSEMR

SEQ ID NO: 6 - protein, 8 aa, Homo sapiens
  MOD_RES (2)..(2): Phosphorylated Tyr
GYMMMFPR

SEQ ID NO: 7 - protein, 13 aa, Homo sapiens
  MOD_RES (5)..(5): Phosphorylated Tyr
SWSSYFSLPNPFR

SEQ ID NO: 8 - protein, 19 aa, Homo sapiens
  MOD_RES (12)..(12): Phosphorylated Tyr
SSPLGQNDNSEYVPMLPGK

SEQ ID NO: 9 - protein, 15 aa, Homo sapiens
  MOD_RES (8)..(8): Phosphorylated Tyr
EADSSSDYVNMDFTK

SEQ ID NO: 10 - protein, 18 aa, Homo sapiens
  MOD_RES (17)..(17): Phosphorylated Tyr
MDAMASPGKDNYRMKSYK

SEQ ID NO: 11 - protein, 13 aa, Homo sapiens
  MOD_RES (11)..(11): Phosphorylated Tyr
DVTIGGSAPIYVK

SEQ ID NO: 12 - protein, 21 aa, Homo sapiens
  MOD_RES (17)..(17): Phosphorylated Tyr
KEQQMKKQPPSEGPSNYDSYK

SEQ ID NO: 13 - protein, 15 aa, Homo sapiens
  MOD_RES (11)..(11): Phosphorylated Tyr
AGYGGSHISGYATLR

SEQ ID NO: 14 - protein, 16 aa, Homo sapiens
  MOD_RES (2)..(2): Phosphorylated Tyr
VYGTIKPAFNQNSAAK

SEQ ID NO: 15 - protein, 15 aa, Homo sapiens
  MOD_RES (13)..(13): Phosphorylated Tyr
ELDRYSLDSEDLYSR

SEQ ID NO: 16 - protein, 30 aa, Homo sapiens
  MOD_RES (23)..(23): Phosphorylated Tyr
AQGPESSPAVPSASSGTAGPGNYVHPLTGR

SEQ ID NO: 17 - protein, 18 aa, Homo sapiens
  MOD_RES (15)..(15): Phosphorylated Tyr
GERITLLRQVDENWYEGR

SEQ ID NO: 18 - protein, 14 aa, Homo sapiens
  MOD_RES (6)..(6): Phosphorylated Tyr
HQYSDYDYHSSSEK

SEQ ID NO: 19 - protein, 22 aa, Homo sapiens
  MOD_RES (20)..(20): Phosphorylated Tyr
DDGMEEVVGHTQGPLDGSLYAK

SEQ ID NO: 20 - protein, 14 aa, Homo sapiens
  MOD_RES (6)..(6): Phosphorylated Tyr
HPAGVYQVSGLHNK

SEQ ID NO: 21 - protein, 13 aa, Homo sapiens
  MOD_RES (7)..(7): Phosphorylated Tyr
HVAYGGYSTPEDR

SEQ ID NO: 22 - protein, 30 aa, Homo sapiens
  MOD_RES (4)..(4): Phosphorylated Tyr
FNKYINTDAKFQVFLKQINSSLVDSNMLVR

SEQ ID NO: 23 - protein, 27 aa, Homo sapiens
  MOD_RES (5)..(5): Phosphorylated Tyr
GGMDYAYIPPPGLQPEPGYGYAPNQGR

SEQ ID NO: 24 - protein, 16 aa, Homo sapiens
  MOD_RES (7)..(7): Phosphorylated Tyr
ESPEGSYTDDANQEVR

SEQ ID NO: 25 - protein, 32 aa, Homo sapiens
  MOD_RES (5)..(5): Phosphorylated Tyr
SLEQYDQVLDGAHGASLTDLANLSEGVSLAER

SEQ ID NO: 26 - protein, 25 aa, Homo sapiens
  MOD_RES (18)..(18): Phosphorylated Tyr
DNVFYYGEEGGGEEDQDYDITQLHR

SEQ ID NO: 27 - protein, 12 aa, Homo sapiens
  MOD_RES (6)..(6): Phosphorylated Tyr
KLADMYGGGEDD

SEQ ID NO: 28 - protein, 24 aa, Homo sapiens
  MOD_RES (2)..(2): Phosphorylated Tyr
TYRYFLLLFWVGQPYPTLSTPLSK

SEQ ID NO: 29 - protein, 24 aa, Homo sapiens
  MOD_RES (4)..(4): Phosphorylated Tyr
TYRYFLLLFWVGQPYPTLSTPLSK

SEQ ID NO: 30 - protein, 13 aa, Homo sapiens
  MOD_RES (6)..(6): Phosphorylated Tyr
KTEGTYDLPYWDR

SEQ ID NO: 31 - protein, 15 aa, Homo sapiens
  MOD_RES (14)..(14): Phosphorylated Tyr
IKENLAVGSKINGYK

SEQ ID NO: 32 - protein, 25 aa, Homo sapiens
  MOD_RES (9)..(9): Phosphorylated Tyr
SESTENQSYAKHSANMNFSNHNNVR

SEQ ID NO: 33 - protein, 10 aa, Homo sapiens
  MOD_RES (4)..(4): Phosphorylated Tyr
KVIYSQPSAR

SEQ ID NO: 34 - protein, 22 aa, Homo sapiens
  MOD_RES (6)..(6): Phosphorylated Tyr
GPHYFYWSREDGRPVPSGTQQR

SEQ ID NO: 35 - protein, 38 aa, Homo sapiens
  MOD_RES (5)..(5): Phosphorylated Tyr
NPLMYLTGVQTDKAGDISCNADINPLKIGQTSSSVSFK

SEQ ID NO: 36 - protein, 18 aa, Homo sapiens
  MOD_RES (6)..(6): Phosphorylated Tyr
EGVIRYVIGVGDAFRSEK

SEQ ID NO: 37 - protein, 12 aa, Homo sapiens
  MOD_RES (11)..(11): Phosphorylated Tyr
ARYEMASNPLYR

SEQ ID NO: 38 - protein, 32 aa, Homo sapiens
  MOD_RES (1)..(1): Phosphorylated Tyr
YSVKDKEDTQVDSEARPMKDETFGEYSDNEEK

SEQ ID NO: 39 - protein, 9 aa, Homo sapiens
  MOD_RES (1)..(1): Phosphorylated Tyr
YELIVDKSR

SEQ ID NO: 40 - protein, 14 aa, Homo sapiens
  MOD_RES (9)..(9): Phosphorylated Tyr
APGDQGEKYIDLRH

SEQ ID NO: 41 - protein, 23 aa, Homo sapiens
  MOD_RES (1)..(1): Phosphorylated Tyr
YARWAGAASSGELSFSLRTNATR

SEQ ID NO: 42 - protein, 23 aa, Homo sapiens
  MOD_RES (11)..(11): Phosphorylated Tyr
SNILWDKEHIYDEQPPNVEEWVK

SEQ ID NO: 43 - protein, 31 aa, Homo sapiens
  MOD_RES (16)..(16): Phosphorylated Tyr
NVSAGTQDVPSPPSDYVERVDSPMAYSSNGK

SEQ ID NO: 44 - protein, 26 aa, Homo sapiens
  MOD_RES (10)..(10): Phosphorylated Tyr
TEQDHYETDYTTGGESCDELEEDWIR

SEQ ID NO: 45 - protein, 10 aa, Homo sapiens
  MOD_RES (9)..(9): Phosphorylated Tyr
NFDTGLQEYK

SEQ ID NO: 46 - protein, 16 aa, Homo sapiens
  MOD_RES (9)..(9): Phosphorylated Tyr
LQDPSQHSYYDSGLEE

SEQ ID NO: 47 - protein, 35 aa, Homo sapiens
  MOD_RES (30)..(30): Phosphorylated Tyr
VESVSCMPTLVALSVISLGSITLVTGMGIYICLRK

SEQ ID NO: 48 - protein, 23 aa, Homo sapiens
  MOD_RES (12)..(12): Phosphorylated Tyr
DLDTGTNGEISYSLYYSSQEIDK

SEQ ID NO: 49 - protein, 23 aa, Homo sapiens
  MOD_RES (15)..(15): Phosphorylated Tyr
DLDTGTNGEISYSLYYSSQEIDK

SEQ ID NO: 50 - protein, 23 aa, Homo sapiens
  MOD_RES (16)..(16): Phosphorylated Tyr
DLDTGTNGEISYSLYYSSQEIDK

SEQ ID NO: 51 - protein, 10 aa, Homo sapiens
  MOD_RES (4)..(4): Phosphorylated Tyr
NLIYDNADNK

SEQ ID NO: 52 - protein, 13 aa, Homo sapiens
  MOD_RES (9)..(9): Phosphorylated Tyr
AKPTGNGIYINGR

SEQ ID NO: 53 - protein, 24 aa, Homo sapiens
  MOD_RES (2)..(2): Phosphorylated Tyr
VYAPASTLVDQPYANEGTVVVTER

SEQ ID NO: 54 - protein, 24 aa, Homo sapiens
  MOD_RES (13)..(13): Phosphorylated Tyr
VYAPASTLVDQPYANEGTVVVTER

SEQ ID NO: 55 - protein, 23 aa, Homo sapiens
  MOD_RES (12)..(12): Phosphorylated Tyr
VLAPASTLQSSYQIPTENSMTAR

SEQ ID NO: 56 - protein, 18 aa, Homo sapiens
  MOD_RES (11)..(11): Phosphorylated Tyr
EITQDTNDITYADLNLPK

SEQ ID NO: 57 - protein, 28 aa, Homo sapiens
  MOD_RES (3)..(3): Phosphorylated Tyr
KQYKKWVELPITFPNLDYSECCLFSDED

SEQ ID NO: 58 - protein, 28 aa, Homo sapiens
  MOD_RES (18)..(18): Phosphorylated Tyr
KQYKKWVELPITFPNLDYSECCLFSDED

SEQ ID NO: 59 - protein, 21 aa, Homo sapiens
  MOD_RES (8)..(8): Phosphorylated Tyr
MTLKVQEYPTLKVPYETLNKR

SEQ ID NO: 60 - protein, 12 aa, Homo sapiens
  MOD_RES (7)..(7): Phosphorylated Tyr
VGSFDPYSDDPR

SEQ ID NO: 61 - protein, 14 aa, Homo sapiens
  MOD_RES (3)..(3): Phosphorylated Tyr
AVYHLATRLVQTAR

SEQ ID NO: 62 - protein, 18 aa, Homo sapiens
  MOD_RES (16)..(16): Phosphorylated Tyr
EFVDFWEKIFQKFSAYQK

SEQ ID NO: 63 - protein, 18 aa, Homo sapiens
  MOD_RES (16)..(16): Phosphorylated Tyr
EAAHPTDVSISKTALYSR

SEQ ID NO: 64 - protein, 11 aa, Homo sapiens
  MOD_RES (8)..(8): Phosphorylated Tyr
GALTGGYYDTR

SEQ ID NO: 65 - protein, 20 aa, Homo sapiens
  MOD_RES (12)..(12): Phosphorylated Tyr
VTEDLFSSLKGYGKRVADIK

SEQ ID NO: 66 - protein, 16 aa, Homo sapiens
  MOD_RES (13)..(13): Phosphorylated Tyr
ICNQHNDPSKTTYISR

SEQ ID NO: 67 - protein, 16 aa, Homo sapiens
  MOD_RES (9)..(9): Phosphorylated Tyr
GYTASQPLYQPSHATE

SEQ ID NO: 68 - protein, 10 aa, Homo sapiens
  MOD_RES (8)..(8): Phosphorylated Tyr
DSQMQNPYSR

SEQ ID NO: 69 - protein, 9 aa, Homo sapiens
  MOD_RES (9)..(9): Phosphorylated Tyr
HSSMPRPDY

SEQ ID NO: 70 - protein, 29 aa, Homo sapiens
  MOD_RES (1)..(1): Phosphorylated Tyr
YLQTALEGLGGVIDAGGETQGYLFPSGLK

SEQ ID NO: 71 - protein, 29 aa, Homo sapiens
  MOD_RES (22)..(22): Phosphorylated Tyr
YLQTALEGLGGVIDAGGETQGYLFPSGLK

SEQ ID NO: 72 - protein, 11 aa, Homo sapiens
  MOD_RES (7)..(7): Phosphorylated Tyr
LMETLMYSRPR

SEQ ID NO: 73 - protein, 15 aa, Homo sapiens
  MOD_RES (11)..(11): Phosphorylated Tyr
KVLVEQTKNEYFELK

SEQ ID NO: 74 - protein, 16 aa, Homo sapiens
  MOD_RES (7)..(7): Phosphorylated Tyr
LEDDPLYTSYSSMMAK

SEQ ID NO: 75 - protein, 21 aa, Homo sapiens
  MOD_RES (16)..(16): Phosphorylated Tyr
LRAAQEMARKLSELPYDGKAR

SEQ ID NO: 76 - protein, 16 aa, Homo sapiens
  MOD_RES (10)..(10): Phosphorylated Tyr
QASEQNWANYSAEQNR

SEQ ID NO: 77 - protein, 16 aa, Homo sapiens
  MOD_RES (2)..(2): Phosphorylated Tyr
GYGNDFPIEDMIPTLK

SEQ ID NO: 78 - protein, 18 aa, Homo sapiens
  MOD_RES (13)..(13): Phosphorylated Tyr
LLKVPVSDLLLSYESPKK

SEQ ID NO: 79 - protein, 14 aa, Homo sapiens
  MOD_RES (4)..(4): Phosphorylated Tyr
AVVYRETDPSPEER

SEQ ID NO: 80 - protein, 29 aa, Homo sapiens
  MOD_RES (27)..(27): Phosphorylated Tyr
EELWKHIQKELVDPSGLSEEQLKEIPYTK

SEQ ID NO: 81 - protein, 15 aa, Homo sapiens
  MOD_RES (1)..(1): Phosphorylated Tyr
YVDKARMVMQTMEPK

SEQ ID NO: 82 - protein, 18 aa, Homo sapiens
  MOD_RES (16)..(16): Phosphorylated Tyr
TDLEMQIESLNEELAYMK

SEQ ID NO: 83 - protein, 12 aa, Homo sapiens
  MOD_RES (4)..(4): Phosphorylated Tyr
TTEYQLSTLEER

SEQ ID NO: 84 - protein, 19 aa, Homo sapiens
  MOD_RES (1)..(1): Phosphorylated Tyr
YEDEINKRTAAENDFVTLK

SEQ ID NO: 85 - protein, 21 aa, Homo sapiens
  MOD_RES (5)..(5): Phosphorylated Tyr
GAFLYEPCGVSTPVLSTGVLR

SEQ ID NO: 86 - protein, 16 aa, Homo sapiens
  MOD_RES (10)..(10): Phosphorylated Tyr
EPDWKCVYTYIQEFYR

SEQ ID NO: 87 - protein, 16 aa, Homo sapiens
  MOD_RES (15)..(15): Phosphorylated Tyr
EPDWKCVYTYIQEFYR

SEQ ID NO: 88 - protein, 20 aa, Homo sapiens
  MOD_RES (4)..(4): Phosphorylated Tyr
GLNYYLPMVEEDEHEPKFEK

SEQ ID NO: 89 - protein, 16 aa, Homo sapiens
  MOD_RES (2)..(2): Phosphorylated Tyr
EYRPCDPQLVSERVAK

SEQ ID NO: 90 - protein, 21 aa, Homo sapiens
  MOD_RES (17)..(17): Phosphorylated Tyr
SWVSLYCVLSKGELGFYKDSK

SEQ ID NO: 91 - protein, 29 aa, Homo sapiens
  MOD_RES (10)..(10): Phosphorylated Tyr
EDMAALEKDYEEVGVDSVEGEGEEEGEEY

SEQ ID NO: 92 - protein, 19 aa, Homo sapiens
  MOD_RES (19)..(19): Phosphorylated Tyr
DYEEVGADSADGEDEGEEY

SEQ ID NO: 93 - protein, 18 aa, Homo sapiens
  MOD_RES (1)..(1): Phosphorylated Tyr
YAHQQPPSPLPVYSSSAK

SEQ ID NO: 94 - protein, 19 aa, Homo sapiens
  MOD_RES (14)..(14): Phosphorylated Tyr
LCEPEVLNSLEETYSPFFR

SEQ ID NO: 95 - protein, 29 aa, Homo sapiens
  MOD_RES (28)..(28): Phosphorylated Tyr
LSKMGESSLRNFTMDTESSVYNFEGEDYR

SEQ ID NO: 96 - protein, 14 aa, Homo sapiens
  MOD_RES (6)..(6): Phosphorylated Tyr
LGQDPYRLGHDPYR

SEQ ID NO: 97 - protein, 32 aa, Homo sapiens
  MOD_RES (31)..(31): Phosphorylated Tyr
EVIAKELSKTYQETPEIDMFLNVATFLDPRYK

SEQ ID NO: 98 - protein, 14 aa, Homo sapiens
  MOD_RES (3)..(3): Phosphorylated Tyr
LFYFKLTDLYKKVK

SEQ ID NO: 99 - protein, 28 aa, Homo sapiens
  MOD_RES (12)..(12): Phosphorylated Tyr
HDAIMDGASPDYVLVEAEANRVAQDALK

SEQ ID NO: 100 - protein, 18 aa, Homo sapiens
  MOD_RES (6)..(6): Phosphorylated Tyr
LGTGNYDVMTPMVDILMK

SEQ ID NO: 101 - protein, 13 aa, Homo sapiens
  MOD_RES (11)..(11): Phosphorylated Tyr
IGSSRGMVSAYPR

SEQ ID NO: 102 - protein, 12 aa, Homo sapiens
  MOD_RES (11)..(11): Phosphorylated Tyr
QFFTPKVLQDYR

SEQ ID NO: 103 - protein, 11 aa, Homo sapiens
  MOD_RES (8)..(8): Phosphorylated Tyr
YVVKTSFYSNK

SEQ ID NO: 104 - protein, 11 aa, Homo sapiens
  MOD_RES (7)..(7): Phosphorylated Tyr
LYGDADYLEER

SEQ ID NO: 105 - protein, 21 aa, Homo sapiens
  MOD_RES (15)..(15): Phosphorylated Tyr
IAELMDKKLPSFGPYLEQRKK

SEQ ID NO: 106 - protein, 24 aa, Homo sapiens
  MOD_RES (8)..(8): Phosphorylated Tyr
DPCFHPGYKKVVNVSDLYKTPCTK

SEQ ID NO: 107 - protein, 16 aa, Homo sapiens
  MOD_RES (15)..(15): Phosphorylated Tyr
DHIFLNSALRATAPYK

SEQ ID NO: 108 - protein, 14 aa, Homo sapiens
  MOD_RES (1)..(1): Phosphorylated Tyr
YELENEEIAAERNK

SEQ ID NO: 109 - protein, 31 aa, Homo sapiens
  MOD_RES (15)..(15): Phosphorylated Tyr
VEALTLRGINSFRQYKYDLVAVGKALEGMFR

SEQ ID NO: 110 - protein, 31 aa, Homo sapiens; identical to SEQ ID NO: 109
  MOD_RES (17)..(17): Phosphorylated Tyr

SEQ ID NO: 111 - protein, 18 aa, Homo sapiens
  MOD_RES (16)..(16): Phosphorylated Tyr
VRGSTGVAAAAGLHRYLR

SEQ ID NO: 112 - protein, 20 aa, Homo sapiens
  MOD_RES (3)..(3): Phosphorylated Tyr
DFYELEPHKFQNKTNGITPR

SEQ ID NO: 113 - protein, 31 aa, Homo sapiens
  MOD_RES (28)..(28): Phosphorylated Tyr
RLSFVDVATGWLGQGLGVACGMAYTGKYFDR

SEQ ID NO: 114 - protein, 12 aa, Homo sapiens
  MOD_RES (8)..(8): Phosphorylated Tyr
SGLSSPIYIDLR

SEQ ID NO: 115 - protein, 9 aa, Homo sapiens
  MOD_RES (8)..(8): Phosphorylated Tyr
LHEEGIIYR

SEQ ID NO: 116 - protein, 30 aa, Homo sapiens
  MOD_RES (24)..(24): Phosphorylated Tyr
AKLGVKANIVDDFQEYNYGTMESYQTEAPR

SEQ ID NO: 117 - protein, 36 aa, Homo sapiens
  MOD_RES (5)..(5): Phosphorylated Tyr
GERGYTGSAGEKGEPGPPGSEGLPGPPGPAGPRGER

SEQ ID NO: 118 - protein, 35 aa, Homo sapiens
  MOD_RES (24)..(24): Phosphorylated Tyr
GDASSIVSAICYTVPKSAMGSSLYALESGSDFKSR

SEQ ID NO: 119 - protein, 18 aa, Homo sapiens
  MOD_RES (16)..(16): Phosphorylated Tyr
DGPTEESALIGHFCGYEK

SEQ ID NO: 120 - protein, 18 aa, Homo sapiens
  MOD_RES (15)..(15): Phosphorylated Tyr
WTVPEGEFDSFVIQYKDR

SEQ ID NO: 121 - protein, 20 aa, Homo sapiens
  MOD_RES (5)..(5): Phosphorylated Tyr
KSDIYVCMISFAHNVAAQGK

SEQ ID NO: 122 - protein, 12 aa, Homo sapiens
  MOD_RES (9)..(9): Phosphorylated Tyr
MKRKKNDIYGED

SEQ ID NO: 123 - protein, 39 aa, Homo sapiens
  MOD_RES (12)..(12): Phosphorylated Tyr
AFMPSILQNETYGALLSGSPPPAQPAAPSTTSAPPLPPR

SEQ ID NO: 124 - protein, 12 aa, Homo sapiens
  MOD_RES (3)..(3): Phosphorylated Tyr
VEYVSSLSSSVR

SEQ ID NO: 125 - protein, 23 aa, Homo sapiens
  MOD_RES (20)..(20): Phosphorylated Tyr
QFCESKNGPPYPQGAGQLDYGSK

SEQ ID NO: 126 - protein, 24 aa, Homo sapiens
  MOD_RES (16)..(16): Phosphorylated Tyr
QSSVTVVSQYDNLEDYHSLPQHQR

SEQ ID NO: 127 - protein, 13 aa, Homo sapiens
  MOD_RES (7)..(7): Phosphorylated Tyr
RVAETDYEMETQR

SEQ ID NO: 128 - protein, 12 aa, Homo sapiens
  MOD_RES (4)..(4): Phosphorylated Tyr
NRLYHSLGPVTR

SEQ ID NO: 129 - protein, 25 aa, Homo sapiens
  MOD_RES (3)..(3): Phosphorylated Tyr
QGYKCKDCGANCHKQCKDLLVLACR

SEQ ID NO: 130 - protein, 17 aa, Homo sapiens
  MOD_RES (7)..(7): Phosphorylated Tyr
SLYVAEYHSEPVEDEKP

SEQ ID NO: 131 - protein, 20 aa, Homo sapiens
  MOD_RES (14)..(14): Phosphorylated Tyr
KSSMDGAQNQDDGYLALSSR

SEQ ID NO: 132 - protein, 20 aa, Homo sapiens
  MOD_RES (14)..(14): Phosphorylated Tyr
THSLSNADGQYDPYTDSRFR

SEQ ID NO: 133 - protein, 21 aa, Homo sapiens
  MOD_RES (16)..(16): Phosphorylated Tyr
YREASARKKIRLDRKYIVSCK

SEQ ID NO: 134 - protein, 20 aa, Homo sapiens
  MOD_RES (9)..(9): Phosphorylated Tyr
EEEETRQMYDMVVKIIDVLR

SEQ ID NO: 135 - protein, 21 aa, Homo sapiens
  MOD_RES (5)..(5): Phosphorylated Tyr
SLPSYLNGVMPPTQSFAPDPK

SEQ ID NO: 136 - protein, 26 aa, Homo sapiens
  MOD_RES (22)..(22): Phosphorylated Tyr
SLPSYLNGVMPPTQSFAPDPKYVSSK

SEQ ID NO: 137 - protein, 33 aa, Homo sapiens
  MOD_RES (27)..(27): Phosphorylated Tyr
TEFDQEIDMGSLNPGKQLFEKMISGMYMGELVR

SEQ ID NO: 138 - protein, 31 aa, Homo sapiens
  MOD_RES (18)..(18): Phosphorylated Tyr
TINPSKYQTIRKAGKVHYPVAWVNTMVFDFK

SEQ ID NO: 139 - protein, 23 aa, Homo sapiens
  MOD_RES (3)..(3): Phosphorylated Tyr
CLYMWPSVPDEKGELLNPTGTVR

SEQ ID NO: 140 - protein, 17 aa, Homo sapiens
  MOD_RES (8)..(8): Phosphorylated Tyr
LYKYHSQYHTVAGNDIK

SEQ ID NO: 141 - protein, 12 aa, Homo sapiens
  MOD_RES (8)..(8): Phosphorylated Tyr
NRYAGEVYGMIR

SEQ ID NO: 142 - protein, 20 aa, Homo sapiens
  MOD_RES (11)..(11): Phosphorylated Tyr
AGSSGNSCITYQPSVSGEHK

SEQ ID NO: 143 - protein, 18 aa, Homo sapiens
  MOD_RES (7)..(7): Phosphorylated Tyr
LEETKEYQEPEVPESNQK

SEQ ID NO: 144 - protein, 14 aa, Homo sapiens
  MOD_RES (12)..(12): Phosphorylated Tyr
STEQSWPHSAPYSR

SEQ ID NO: 145 - protein, 14 aa, Homo sapiens
  MOD_RES (2)..(2): Phosphorylated Tyr
MYAPAWVAPEALQK

SEQ ID NO: 146 - protein, 20 aa, Homo sapiens
  MOD_RES (15)..(15): Phosphorylated Tyr
TQTVRGTLAYLPEEYIKTGR

SEQ ID NO: 147 - protein, 14 aa, Homo sapiens
  MOD_RES (4)..(4): Phosphorylated Tyr
ISSYPEDNFPDEEK

SEQ ID NO: 148 - protein, 17 aa, Homo sapiens
  MOD_RES (9)..(9): Phosphorylated Tyr
FVDDVNNNYYEAPSCPR

SEQ ID NO: 149 - protein, 17 aa, Homo sapiens
  MOD_RES (1)..(1): Phosphorylated Tyr
YAAVKIHQLNKSWRDEK

SEQ ID NO: 150 - protein, 20 aa, Homo sapiens
  MOD_RES (9)..(9): Phosphorylated Tyr
LRMINEFGYCSLDYGVAYSR

SEQ ID NO: 151 - protein, 12 aa, Homo sapiens
  MOD_RES (4)..(4): Phosphorylated Tyr
DESYEELLRKTK

SEQ ID NO: 152 - protein, 23 aa, Homo sapiens
  MOD_RES (6)..(6): Phosphorylated Tyr
EIEPNYESPSSNNQDKDSSQASK

SEQ ID NO: 153 - protein, 17 aa, Homo sapiens
  MOD_RES (6)..(6): Phosphorylated Tyr
SSAIRYQEVWTSSTSPR

SEQ ID NO: 154 - protein, 21 aa, Homo sapiens
  MOD_RES (14)..(14): Phosphorylated Tyr
NAIKVPIVINPNAYDNLAIYK

SEQ ID NO: 155 - protein, 21 aa, Homo sapiens
  MOD_RES (20)..(20): Phosphorylated Tyr
NAIKVPIVINPNAYDNLAIYK

SEQ ID NO: 156 - protein, 17 aa, Homo sapiens
  MOD_RES (9)..(9): Phosphorylated Tyr
TTSVISHTYEEIETESK

SEQ ID NO: 157 - protein, 18 aa, Homo sapiens
  MOD_RES (8)..(8): Phosphorylated Tyr
ACSVEELYAIPPDADVAK

SEQ ID NO: 158 - protein, 14 aa, Homo sapiens
  MOD_RES (6)..(6): Phosphorylated Tyr
STSSPYHAGNLLQR

SEQ ID NO: 159 - protein, 20 aa, Homo sapiens
  MOD_RES (15)..(15): Phosphorylated Tyr
ILEHYQWDLSAASRYVLARP

SEQ ID NO: 160 - protein, 15 aa, Homo sapiens
  MOD_RES (9)..(9): Phosphorylated Tyr
LLDDFDGTYETQGGK

SEQ ID NO: 161 - protein, 10 aa, Homo sapiens
  MOD_RES (4)..(4): Phosphorylated Tyr
LQQYIAPGMK

SEQ ID NO: 162 - protein, 15 aa, Homo sapiens
  MOD_RES (4)..(4): Phosphorylated Tyr
QPHYSAFGSVGEWLR

SEQ ID NO: 163 - protein, 10 aa, Homo sapiens
  MOD_RES (3)..(3): Phosphorylated Tyr
DIYKNPDYVR

SEQ ID NO: 164 - protein, 24 aa, Homo sapiens
  MOD_RES (15)..(15): Phosphorylated Tyr
QLLRFASDAANGMQYLSEKQFIHR

SEQ ID NO: 165 - protein, 16 aa, Homo sapiens
  MOD_RES (11)..(11): Phosphorylated Tyr
GGNRPNTGPLYTEADR

SEQ ID NO: 166 - protein, 27 aa, Homo sapiens
  MOD_RES (7)..(7): Phosphorylated Tyr
PLIFNTYQCYLKDAYDNVTLDVELARR

SEQ ID NO: 167 - protein, 11 aa, Homo sapiens
  MOD_RES (1)..(1): Phosphorylated Tyr
YLLQEQLKIIK

SEQ ID NO: 168 - protein, 22 aa, Homo sapiens
  MOD_RES (21)..(21): Phosphorylated Tyr
YRNTWDCGLQILKKEGLKAFYK

SEQ ID NO: 169 - protein, 11 aa, Homo sapiens
  MOD_RES (7)..(7): Phosphorylated Tyr
AAYFGIYDTAK

SEQ ID NO: 170 - protein, 19 aa, Homo sapiens
  MOD_RES (4)..(4): Phosphorylated Tyr
ILSYNRANRVVAILCNHQR

SEQ ID NO: 171 - protein, 16 aa, Homo sapiens
  MOD_RES (11)..(11): Phosphorylated Tyr
KNYMSNPSYNYEIVNR

SEQ ID NO: 172 - protein, 20 aa, Homo sapiens
  MOD_RES (7)..(7): Phosphorylated Tyr
DMHDWLYAFNPLLAGTIRSK

SEQ ID NO: 173 - protein, 13 aa, Homo sapiens
  MOD_RES (1)..(1): Phosphorylated Tyr
YANRVKKLNVDVR

SEQ ID NO: 174 - protein, 11 aa, Homo sapiens
  MOD_RES (8)..(8): Phosphorylated Tyr
ESIFCIQYNVR

SEQ ID NO: 175 - protein, 17 aa, Homo sapiens
  MOD_RES (6)..(6): Phosphorylated Tyr
TFHIFYQLLSGAGEHLK

SEQ ID NO: 176 - protein, 17 aa, Homo sapiens
  MOD_RES (10)..(10): Phosphorylated Tyr
HDCDLLREQYEEEQEAK

SEQ ID NO: 177 - protein, 37 aa, Homo sapiens
  MOD_RES (13)..(13): Phosphorylated Tyr
ALCYPRVKVGNEYVTKGQTVEQVSNAVGALAKAVYEK

SEQ ID NO: 178 - protein, 12 aa, Homo sapiens
  MOD_RES (2)..(2): Phosphorylated Tyr
SYHIFYQILSNK

SEQ ID NO: 179 - protein, 12 aa, Homo sapiens
  MOD_RES (6)..(6): Phosphorylated Tyr
SYHIFYQILSNK

SEQ ID NO: 180 - protein, 14 aa, Homo sapiens
  MOD_RES (3)..(3): Phosphorylated Tyr
AAYLTSLNSADLLK

SEQ ID NO: 181 - protein, 15 aa, Homo sapiens
  MOD_RES (3)..(3): Phosphorylated Tyr
QKYEETQAELEASQK

SEQ ID NO: 182 - protein, 11 aa, Homo sapiens
  MOD_RES (4)..(4): Phosphorylated Tyr
ELTYQTEEDRK

SEQ ID NO: 183 - protein, 31 aa, Homo sapiens
  MOD_RES (3)..(3): Phosphorylated Tyr
HLYKMDPTKQYKVMKTIPLYNLTGLSVSNGK

SEQ ID NO: 184 - protein, 31 aa, Homo sapiens; identical to SEQ ID NO: 183
  MOD_RES (11)..(11): Phosphorylated Tyr

SEQ ID NO: 185 - protein, 31 aa, Homo sapiens; identical to SEQ ID NO: 183
  MOD_RES (20)..(20): Phosphorylated Tyr

SEQ ID NO: 186 - protein, 14 aa, Homo sapiens
  MOD_RES (3)..(3): Phosphorylated Tyr
NQYVPYPHAPGSQR

SEQ ID NO: 187 - protein, 13 aa, Homo sapiens
  MOD_RES (3)..(3): Phosphorylated Tyr
SLYTSMARPPLPR

SEQ ID NO: 188 - protein, 16 aa, Homo sapiens
  MOD_RES (1)..(1): Phosphorylated Tyr
YKIRRAATIVLQSYLR

SEQ ID NO: 189 - protein, 13 aa, Homo sapiens
  MOD_RES (3)..(3): Phosphorylated Tyr
VEYLSDGFLEKNR

SEQ ID NO: 190 - protein, 18 aa, Homo sapiens
  MOD_RES (16)..(16): Phosphorylated Tyr
HTSCTVSDLIVGNEYYFR

SEQ ID NO: 191 - protein, 35 aa, Homo sapiens
  MOD_RES (5)..(5): Phosphorylated Tyr
ANPQYTVYSQASTMSIPVAMETDGPLFEDVQMLRK

SEQ ID NO: 192 - protein, 15 aa, Homo sapiens
  MOD_RES (10)..(10): Phosphorylated Tyr
HYQLDQLPDYYDTPL

SEQ ID NO: 193 - protein, 18 aa, Homo sapiens
  MOD_RES (1)..(1): Phosphorylated Tyr
YGQFSGLNPGGRPITPPR

SEQ ID NO: 194 - protein, 16 aa, Homo sapiens
  MOD_RES (3)..(3): Phosphorylated Tyr
LLYSRKEGQRQENKNK

SEQ ID NO: 195 - protein, 24 aa, Homo sapiens
  MOD_RES (1)..(1): Phosphorylated Tyr
YECVATNSAGVRYSSPANLYVRVR

SEQ ID NO: 196 - protein, 18 aa, Homo sapiens
  MOD_RES (17)..(17): Phosphorylated Tyr
TTTGTWAETHIVDSPNYK

SEQ ID NO: 197 - protein, 16 aa, Homo sapiens
  MOD_RES (4)..(4): Phosphorylated Tyr
CVRYWPDDTEVYGDIK

SEQ ID NO: 198 - protein, 34 aa, Homo sapiens
  MOD_RES (9)..(9): Phosphorylated Tyr
YWPDDTEVYGDIKVTLIETEPLAEYVIRTFTVQK

SEQ ID NO: 199 - protein, 15 aa, Homo sapiens
  MOD_RES (2)..(2): Phosphorylated Tyr
LYLPKNELDNLHKQK

SEQ ID NO: 200 - protein, 17 aa, Homo sapiens
  MOD_RES (8)..(8): Phosphorylated Tyr
SYLNCERYSIGLLDMTK

SEQ ID NO: 201 - protein, 13 aa, Homo sapiens
  MOD_RES (3)..(3): Phosphorylated Tyr
ACYRDMSSFPETK

SEQ ID NO: 202 - protein, 21 aa, Homo sapiens
  MOD_RES (5)..(5): Phosphorylated Tyr
FANEYPNITRLYSLGKSVESR

SEQ ID NO: 203 - protein, 13 aa, Homo sapiens
  MOD_RES (8)..(8): Phosphorylated Tyr
LRQHHDEYEDEIR

SEQ ID NO: 204 - protein, 23 aa, Homo sapiens
  MOD_RES (19)..(19): Phosphorylated Tyr
SLLSHEFQDETDTEEETLYSSKH

SEQ ID NO: 205 - protein, 31 aa, Homo sapiens
  MOD_RES (23)..(23): Phosphorylated Tyr
PISVWQGIPASPKGAFLSNDAAYTYFYKGTK

SEQ ID NO: 206 - protein, 31 aa, Homo sapiens; identical to SEQ ID NO: 205
  MOD_RES (25)..(25): Phosphorylated Tyr

SEQ ID NO: 207 - protein, 13 aa, Homo sapiens
  MOD_RES (11)..(11): Phosphorylated Tyr
LQEESDYITHYTR

SEQ ID NO: 208 - protein, 15 aa, Homo sapiens
  MOD_RES (1)..(1): Phosphorylated Tyr
YEPNPHYHENAVIQK

SEQ ID NO: 209 - protein, 17 aa, Homo sapiens
  MOD_RES (12)..(12): Phosphorylated Tyr
FGMSEKLGVMTYSDTGK

SEQ ID NO: 210 - protein, 14 aa, Homo sapiens
  MOD_RES (13)..(13): Phosphorylated Tyr
MDTCSSNLNNSIYK

SEQ ID NO: 211 - protein, 13 aa, Homo sapiens
  MOD_RES (12)..(12): Phosphorylated Tyr
KMNTWLGIFYGYK

SEQ ID NO: 212 - protein, 10 aa, Homo sapiens
  MOD_RES (8)..(8): Phosphorylated Tyr
RSENEDIYYK

SEQ ID NO: 213 - protein, 25 aa, Homo sapiens
  MOD_RES (10)..(10): Phosphorylated Tyr
ELDKMISVFYTAVTPMLNPIIYSLR

SEQ ID NO: 214 - protein, 25 aa, Homo sapiens
  MOD_RES (22)..(22): Phosphorylated Tyr
ELDKMISVFYTAVTPMLNPIIYSLR

SEQ ID NO: 215 - protein, 24 aa, Homo sapiens
  MOD_RES (9)..(9): Phosphorylated Tyr
ITAVASVMYTVVPQMMNPFIYSLR

SEQ ID NO: 216 - protein, 15 aa, Homo sapiens
  MOD_RES (1)..(1): Phosphorylated Tyr
YLGIMKPLTYPMRQK

SEQ ID NO: 217 - protein, 36 aa, Homo sapiens
  MOD_RES (2)..(2): Phosphorylated Tyr
TYSVGVCTFAVGPEQGGCKDGGVCLLSGTKGASFGR

SEQ ID NO: 218 - protein, 27 aa, Homo sapiens
  MOD_RES (2)..(2): Phosphorylated Tyr
LYWTDGNTINMANMDGSNSKILFQNQK

SEQ ID NO: 219 - protein, 21 aa, Homo sapiens
  MOD_RES (10)..(10): Phosphorylated Tyr
SQYLSAEENYESCPPSPYTER

SEQ ID NO: 220 - protein, 27 aa, Homo sapiens
  MOD_RES (2)..(2): Phosphorylated Tyr
AYAASPTSITVTWETPVSGNGEIQNYK

SEQ ID NO: 221 - protein, 27 aa, Homo sapiens
  MOD_RES (26)..(26): Phosphorylated Tyr
AYAASPTSITVTWETPVSGNGEIQNYK

SEQ ID NO: 222 - protein, 12 aa, Homo sapiens
  MOD_RES (9)..(9): Phosphorylated Tyr
DKLNTQSTYSEA

SEQ ID NO: 223 - protein, 23 aa, Homo sapiens
  MOD_RES (9)..(9): Phosphorylated Tyr
SPVCMEFQYQATGGRGVALQVVR

SEQ ID NO: 224 - protein, 26 aa, Homo sapiens
  MOD_RES (23)..(23): Phosphorylated Tyr
YYLAVDPVSGSLYVSDTNSRRIYRVK

SEQ ID NO: 225 - protein, 30 aa, Homo sapiens
  MOD_RES (22)..(22): Phosphorylated Tyr
HAVQTTLESATAIAVSYSGVLYITETDEKK

SEQ ID NO: 226 - protein, 23 aa, Homo sapiens
  MOD_RES (22)..(22): Phosphorylated Tyr
TWSYTYLEKAGVCLPASLALPYR

SEQ ID NO: 227 - protein, 34 aa, Homo sapiens
  MOD_RES (11)..(11): Phosphorylated Tyr
QILYTAYGEIYMDTNPNFQIIIGYHGGLYDPLTK

SEQ ID NO: 228 - protein, 18 aa, Homo sapiens
  MOD_RES (14)..(14): Phosphorylated Tyr
DLPSLPGGPRESSYMEMK

SEQ ID NO: 229 - protein, 15 aa, Homo sapiens
  MOD_RES (5)..(5): Phosphorylated Tyr
QTSAYNISNSSTFTK

SEQ ID NO: 230 - protein, 20 aa, Homo sapiens
  MOD_RES (3)..(3): Phosphorylated Tyr
EMYLTKLLSTKVAIHSVLEK

SEQ ID NO: 231 - protein, 24 aa, Homo sapiens
  MOD_RES (8)..(8): Phosphorylated Tyr
KLNTLAHYKIPEGASLAMSLIDKK

SEQ ID NO: 232 - protein, 13 aa, Homo sapiens
  MOD_RES (7)..(7): Phosphorylated Tyr
KKDEGSYSLEEPK

SEQ ID NO: 233 - protein, 18 aa, Homo sapiens
  MOD_RES (7)..(7): Phosphorylated Tyr
QANGGAYQKPTKQEEFYA

SEQ ID NO: 234 - protein, 17 aa, Homo sapiens
  MOD_RES (16)..(16): Phosphorylated Tyr
QASVTYQKPDKQEEFYA

SEQ ID NO: 235 - protein, 17 aa, Homo sapiens
  MOD_RES (14)..(14): Phosphorylated Tyr
SSEVDVSDLGSRNYSAR

SEQ ID NO: 236 - protein, 15 aa, Homo sapiens
  MOD_RES (8)..(8): Phosphorylated Tyr
ENDTITIYSTINHSK

SEQ ID NO: 237 - protein, 16 aa, Homo sapiens
  MOD_RES (3)..(3): Phosphorylated Tyr
EMYELQTFTELNEESR

SEQ ID NO: 238 - protein, 11 aa, Homo sapiens
  MOD_RES (5)..(5): Phosphorylated Tyr
DSGLYKDLLHK

SEQ ID NO: 239 - protein, 27 aa, Homo sapiens
  MOD_RES (2)..(2): Phosphorylated Tyr
VYAADPYHHALAPAPTYGVGAMASIYR

SEQ ID NO: 240 - protein, 27 aa, Homo sapiens
  MOD_RES (7)..(7): Phosphorylated Tyr
VYAADPYHHALAPAPTYGVGAMASIYR

SEQ ID NO: 241 - protein, 32 aa, Homo sapiens
  MOD_RES (30)..(30): Phosphorylated Tyr
HQGLGGTLPPRTFINRNAAGTGRMSAPRNYSR

SEQ ID NO: 242 - protein, 28 aa, Homo sapiens
  MOD_RES (13)..(13): Phosphorylated Tyr
SLGTGAPVIESPYGETISPEDAPESISK

SEQ ID NO: 243 - protein, 20 aa, Homo sapiens
  MOD_RES (5)..(5): Phosphorylated Tyr
FWKLYIEAEIKAKNYDKVEK

SEQ ID NO: 244 - protein, 32 aa, Homo sapiens
  MOD_RES (7)..(7): Phosphorylated Tyr
DPDSNPYSLLDNTESDQTADTDASESHHSTNR

SEQ ID NO: 245 - protein, 33 aa, Homo sapiens
  MOD_RES (21)..(21): Phosphorylated Tyr
KCPYSVPFYPTFKEGMALEDYQRMLGYQVKDSK

SEQ ID NO: 246 - protein, 14 aa, Homo sapiens
  MOD_RES (7)..(7): Phosphorylated Tyr
STAYEDYYYHPPPR

SEQ ID NO: 247 - protein, 35 aa, Homo sapiens
  MOD_RES (9)..(9): Phosphorylated Tyr
PKNENPVDYTVQIPPSTTYAITPMKRPMEEDGEEK

SEQ ID NO: 248 - protein, 35 aa, Homo sapiens; identical to SEQ ID NO: 247
  MOD_RES (19)..(19): Phosphorylated Tyr

SEQ ID NO: 249 - protein, 23 aa, Homo sapiens
  MOD_RES (1)..(1): Phosphorylated Tyr
YLKAALYVGDLDPDVTEDMLYKK

SEQ ID NO: 250 - protein, 35 aa, Homo sapiens
  MOD_RES (13)..(13): Phosphorylated Tyr
SNGTNTSAPQDIYAVNGIAFHPVHGTLATVGSDGR

SEQ ID NO: 251 - protein, 20 aa, Homo sapiens
  MOD_RES (8)..(8): Phosphorylated Tyr
LPDAHSDYARYSGSYNDYLR

SEQ ID NO: 252 - protein, 20 aa, Homo sapiens
  MOD_RES (11)..(11): Phosphorylated Tyr
LPDAHSDYARYSGSYNDYLR

SEQ ID NO: 253 - protein, 32 aa, Homo sapiens
  MOD_RES (18)..(18): Phosphorylated Tyr
LPDAHSDYARYSGSYNDYLRAAQMHSGYQRRM

SEQ ID NO: 254 - protein, 17 aa, Homo sapiens
  MOD_RES (2)..(2): Phosphorylated Tyr
YYDSRPGGYGYGYGRSR

SEQ ID NO: 255 - protein, 25 aa, Homo sapiens
  MOD_RES (4)..(4): Phosphorylated Tyr
RSLYALFSQFGHVVDIVALKTMKMR

SEQ ID NO: 256 - protein, 32 aa, Homo sapiens
  MOD_RES (3)..(3): Phosphorylated Tyr
GGYEDPYYGYEDFQVGARGRGGRGARGAAPSR

SEQ ID NO: 257 - protein, 33 aa, Homo sapiens
  MOD_RES (17)..(17): Phosphorylated Tyr
GDQGEPGPSGNPGKVGYPGPSGPLGARGIPGIK

SEQ ID NO: 258 - protein, 19 aa, Homo sapiens
  MOD_RES (14)..(14): Phosphorylated Tyr
SQREDEEEEEGENYQKGER

SEQ ID NO: 259 - protein, 19 aa, Homo sapiens
  MOD_RES (16)..(16): Phosphorylated Tyr
GYPGVQAPEDLEWERYRGR

SEQ ID NO: 260 - protein, 24 aa, Homo sapiens
  MOD_RES (13)..(13): Phosphorylated Tyr
FSSLYISQFIIMYSLDGKKWQTYR

SEQ ID NO: 261 - protein, 24 aa, Homo sapiens
  MOD_RES (23)..(23): Phosphorylated Tyr
FSSLYISQFIIMYSLDGKKWQTYR

SEQ ID NO: 262 - protein, 22 aa, Homo sapiens
  MOD_RES (9)..(9): Phosphorylated Tyr
NSHQNKGHYQNVVEVREEHSSK

SEQ ID NO: 263 - protein, 32 aa, Homo sapiens
  MOD_RES (3)..(3): Phosphorylated Tyr
PIYPCRWLCEAVRDSCEPVMQFFGFYWPEMLK

SEQ ID NO: 264 - protein, 24 aa, Homo sapiens
  MOD_RES (21)..(21): Phosphorylated Tyr
NLEVMDSVRRGAQLAIEECQYQFR

SEQ ID NO: 265 - protein, 29 aa, Homo sapiens
  MOD_RES (25)..(25): Phosphorylated Tyr
YLSTPDRIDLAESLGLSQLQVKTWYQNRR

SEQ ID NO: 266 - protein, 14 aa, Homo sapiens
  MOD_RES (3)..(3): Phosphorylated Tyr
MIYEESKMNLEQER

SEQ ID NO: 267 - protein, 13 aa, Homo sapiens
  MOD_RES (5)..(5): Phosphorylated Tyr
SASPYHGFTIVNR

SEQ ID NO: 268 - protein, 29 aa, Homo sapiens
AEMQLMSPLQISDPFGSFPHSPTMDNYPK

SEQ ID NO: 269 - protein, 40 aa, Homo sapiens
  MOD_RES (33)..(33): Phosphorylated Tyr
EPGGYAAAGSGGAGGVSGGGSSLAAMGGREPQYSSLSAAR

SEQ ID NO: 270 - protein, 23 aa, Homo sapiens
  MOD_RES (7)..(7): Phosphorylated Tyr
RDGTGHYLCNACGLYSKMNGLSR

SEQ ID NO: 271 - protein, 22 aa, Homo sapiens
  MOD_RES (4)..(4): Phosphorylated Tyr
HGKYCHLRGGGGGGGGYAPYGR

SEQ ID NO: 272 - protein, 22 aa, Homo sapiens
  MOD_RES (17)..(17): Phosphorylated Tyr
HGKYCHLRGGGGGGGGYAPYGR

SEQ ID NO: 273 - protein, 22 aa, Homo sapiens
  MOD_RES (20)..(20): Phosphorylated Tyr
HGKYCHLRGGGGGGGGYAPYGR

SEQ ID NO: 274 - protein, 17 aa, Homo sapiens
  MOD_RES (14)..(14): Phosphorylated Tyr
ATGPSSAPSAPPSYEET

SEQ ID NO: 275 - protein, 15 aa, Homo sapiens
  MOD_RES (8)..(8): Phosphorylated Tyr
RQADSCPYGTMYLSP

SEQ ID NO: 276 - protein, 31 aa, Homo sapiens
  MOD_RES (12)..(12): Phosphorylated Tyr
PPHSQTSGSCYYHVISKVPRIRTPSYSPTQR

SEQ ID NO: 277 - protein, 18 aa, Homo sapiens
  MOD_RES (13)..(13): Phosphorylated Tyr
KDVTALKIMKVNYEQIVK

SEQ ID NO: 278 - protein, 20 aa, Homo sapiens
  MOD_RES (8)..(8): Phosphorylated Tyr
RNCYEGAYYNEAPSEPRPGK

SEQ ID NO: 279 - protein, 20 aa, Homo sapiens
  MOD_RES (6)..(6): Phosphorylated Tyr
RKRSQYQRFTYLPANVPIIK

SEQ ID NO: 280 - protein, 24 aa, Homo sapiens
  MOD_RES (8)..(8): Phosphorylated Tyr
HSMGPGGYGDNLGGGQMYSPREMR

SEQ ID NO: 281 - protein, 25 aa, Homo sapiens
  MOD_RES (12)..(12): Phosphorylated Tyr
DHQPAPYSAVPYKFFPEPSGLHEKR

SEQ ID NO: 282 - protein, 28 aa, Homo sapiens
  MOD_RES (22)..(22): Phosphorylated Tyr
QRTHFTSQQLQELEATFQRNRYPDMSTR

SEQ ID NO: 283 - protein, 20 aa, Homo sapiens
  MOD_RES (13)..(13): Phosphorylated Tyr
SIFNSAMQEMEVYVENIRRK

SEQ ID NO: 284 - protein, 20 aa, Homo sapiens
  MOD_RES (18)..(18): Phosphorylated Tyr
THSLHVQQPPPPQQPLAYPK

SEQ ID NO: 285 - protein, 33 aa, Homo sapiens
  MOD_RES (32)..(32): Phosphorylated Tyr
LGTLLPEFPNVKDLNLPASLPEEKVSTFIMMYR

SEQ ID NO: 286 - protein, 35 aa, Homo sapiens
  MOD_RES (13)..(13): Phosphorylated Tyr
MHYPGAMSAAFPYSATPSGTSISSLSVAGMPATSR

SEQ ID NO: 287 - protein, 13 aa, Homo sapiens
  MOD_RES (7)..(7): Phosphorylated Tyr
LQHMQDYPNYKYR

SEQ ID NO: 288 - protein, 13 aa, Homo sapiens
  MOD_RES (10)..(10): Phosphorylated Tyr
LQHMQDYPNYKYR

SEQ ID NO: 289 - protein, 41 aa, Homo sapiens
  MOD_RES (38)..(38): Phosphorylated Tyr
MHFSTVTRDMEAFTASSLSSLGAAGGFPGAASPGADPYGPR

SEQ ID NO: 290 - protein, 25 aa, Homo sapiens
  MOD_RES (10)..(10): Phosphorylated Tyr
VTGLNPKTKYILLMDIVPADDHRYK

SEQ ID NO: 291 - protein, 25 aa, Homo sapiens
  MOD_RES (24)..(24): Phosphorylated Tyr
VTGLNPKTKYILLMDIVPADDHRYK

SEQ ID NO: 292 - protein, 22 aa, Homo sapiens
  MOD_RES (11)..(11): Phosphorylated Tyr
KVPPGLPSSVYAPSPNSDDFNR

SEQ ID NO: 293 - protein, 16 aa, Homo sapiens
  MOD_RES (8)..(8): Phosphorylated Tyr
ECGKAFSYSSDVIQHR

SEQ ID NO: 294 - protein, 9 aa, Homo sapiens
  MOD_RES (3)..(3): Phosphorylated Tyr
HNYYFINYR

SEQ ID NO: 295 - protein, 21 aa, Homo sapiens
  MOD_RES (2)..(2): Phosphorylated Tyr
QYLSTNLVSHIEEMLQTAYNK

SEQ ID NO: 296 - protein, 21 aa, Homo sapiens
  MOD_RES (19)..(19): Phosphorylated Tyr
QYLSTNLVSHIEEMLQTAYNK

SEQ ID NO: 297 - protein, 27 aa, Homo sapiens
  MOD_RES (24)..(24): Phosphorylated Tyr
VLLPFIAYYMITGPWRSLWIRFGYDPR

SEQ ID NO: 298 - protein, 17 aa, Homo sapiens
  MOD_RES (9)..(9): Phosphorylated Tyr
GIIKQFLGYVPIMVKSK

SEQ ID NO: 299 - protein, 12 aa, Homo sapiens
  MOD_RES (1)..(1): Phosphorylated Tyr
YFVAELAAMNIK

SEQ ID NO: 300 - protein, 33 aa, Homo sapiens
  MOD_RES (18)..(18): Phosphorylated Tyr
QVEDFAMIPAKEAKDMLYKMLSENFMSLQEIPK

SEQ ID NO: 301 - protein, 16 aa, Homo sapiens
  MOD_RES (14)..(14): Phosphorylated Tyr
LLRDVYAKDGRVSYPK

SEQ ID NO: 302 - protein, 9 aa, Homo sapiens
  MOD_RES (4)..(4): Phosphorylated Tyr
VMIYQDEVK

SEQ ID NO: 303 - protein, 12 aa, Homo sapiens
  MOD_RES (10)..(10): Phosphorylated Tyr
KSFTPDHVVYAR

SEQ ID NO: 304 - protein, 25 aa, Homo sapiens
  MOD_RES (5)..(5): Phosphorylated Tyr
HYVMYYEMSYGLNIEMHKQAEIVKR

SEQ ID NO: 305 - protein, 25 aa, Homo sapiens
  MOD_RES (10)..(10): Phosphorylated Tyr
HYVMYYEMSYGLNIEMHKQAEIVKR

SEQ ID NO: 306 - protein, 30 aa, Homo sapiens
  MOD_RES (5)..(5): Phosphorylated Tyr
PPVEYDSDFMLESSESQMSFSQSPFLSIAK

SEQ ID NO: 307 - protein, 21 aa, Homo sapiens
  MOD_RES (14)..(14): Phosphorylated Tyr
LLLSYGADPTLATYSGRTIMK

SEQ ID NO: 308 - protein, 24 aa, Homo sapiens
  MOD_RES (17)..(17): Phosphorylated Tyr
LEEEEAEVKRKATDAAYQARQAVK

SEQ ID NO: 309 - protein, 14 aa, Homo sapiens
  MOD_RES (1)..(1): Phosphorylated Tyr
YESQTSFGSMYPTR

SEQ ID NO: 310 - protein, 14 aa, Homo sapiens
  MOD_RES (11)..(11): Phosphorylated Tyr
YESQTSFGSMYPTR

SEQ ID NO: 311 - protein, 30 aa, Homo sapiens
  MOD_RES (12)..(12): Phosphorylated Tyr
LKASRLFQPVQYGQKPEGRTVAFPSTHPPR

SEQ ID NO: 312 - protein, 10 aa, Homo sapiens
  MOD_RES (4)..(4): Phosphorylated Tyr
LKFYYNPNFK

SEQ ID NO: 313 - protein, 10 aa, Homo sapiens
  MOD_RES (5)..(5): Phosphorylated Tyr
LKFYYNPNFK

SEQ ID NO: 314 - protein, 17 aa, Homo sapiens
  MOD_RES (1)..(1): Phosphorylated Tyr
YKPGKGGVPAHMFGVTK

SEQ ID NO: 315 - protein, 18 aa, Homo sapiens
  MOD_RES (7)..(7): Phosphorylated Tyr
VGSRLGYLPGKGTGSLLK

SEQ ID NO: 316 - protein, 24 aa, Homo sapiens
  MOD_RES (1)..(1): Phosphorylated Tyr
YHQYIPPDQKGEKNEPQMDSNYAR

SEQ ID NO: 317 - protein, 15 aa, Homo sapiens
  MOD_RES (12)..(12): Phosphorylated Tyr
MNWIDAPGDVFYMPK

SEQ ID NO: 318 - protein, 9 aa, Homo sapiens
  MOD_RES (7)..(7): Phosphorylated Tyr
REELAPYPK

SEQ ID NO: 319 - protein, 22 aa, Homo sapiens
  MOD_RES (17)..(17): Phosphorylated Tyr
AVSRKDEELDPMDPSSYSDAPR

SEQ ID NO: 320 - protein, 32 aa, Homo sapiens
  MOD_RES (1)..(1): Phosphorylated Tyr
YHEDAHMLDTQYRMHEGICAFPSVAFYKSKLK

SEQ ID NO: 321 - protein, 32 aa, Homo sapiens; identical to SEQ ID NO: 320
  MOD_RES (27)..(27): Phosphorylated Tyr

SEQ ID NO: 322 - protein, 15 aa, Homo sapiens
  MOD_RES (6)..(6): Phosphorylated Tyr
HQEPVYSVAFSPDGR

SEQ ID NO: 323 - protein, 14 aa, Homo sapiens
  MOD_RES (11)..(11): Phosphorylated Tyr
PFRGSQSPKRYKLR

SEQ ID NO: 324 - protein, 23 aa, Homo sapiens
  MOD_RES (3)..(3): Phosphorylated Tyr
GPYRIYDPGGSVPSGEASAAFER

SEQ ID NO: 325 - protein, 23 aa, Homo sapiens
  MOD_RES (6)..(6): Phosphorylated Tyr
GPYRIYDPGGSVPSGEASAAFER

SEQ ID NO: 326 - protein, 15 aa, Homo sapiens
  MOD_RES (8)..(8): Phosphorylated Tyr
QQHPENIYSNSQGKP

SEQ ID NO: 327 - protein, 16 aa, Homo sapiens
  MOD_RES (1)..(1): Phosphorylated Tyr
YFLNHIDQTTTWQDPR

SEQ ID NO: 328 - protein, 31 aa, Homo sapiens
  MOD_RES (14)..(14): Phosphorylated Tyr
HDDHYELIVDGRVYYICIVCKRSYVCLTSLR

SEQ ID NO: 329 - protein, 31 aa, Homo sapiens; identical to SEQ ID NO: 328
  MOD_RES (24)..(24): Phosphorylated Tyr

SEQ ID NO: 330 - protein, 18 aa, Homo sapiens
  MOD_RES (1)..(1): Phosphorylated Tyr
YVMKTDTDVFINTGNLVK

SEQ ID NO: 331 - protein, 25 aa, Homo sapiens
  MOD_RES (4)..(4): Phosphorylated Tyr
GAAYGADRPFHLSARDAREAVHAWR

SEQ ID NO: 332 - protein, 19 aa, Homo sapiens
  MOD_RES (4)..(4): Phosphorylated Tyr
NALYHVHNGEDVVLLTTCK

SEQ ID NO: 333 - protein, 20 aa, Homo sapiens
  MOD_RES (19)..(19): Phosphorylated Tyr
LIVTKQIGGDGMMDITDTYK

SEQ ID NO: 334 - protein, 16 aa, Homo sapiens
  MOD_RES (1)..(1): Phosphorylated Tyr
YRYPGSLDESQMAKHR

SEQ ID NO: 335 - protein, 21 aa, Homo sapiens
  MOD_RES (14)..(14): Phosphorylated Tyr
QNIDAGERPCLQGYYTAAELK

SEQ ID NO: 336 - protein, 21 aa, Homo sapiens
  MOD_RES (15)..(15): Phosphorylated Tyr
QNIDAGERPCLQGYYTAAELK

SEQ ID NO: 337 - protein, 11 aa, Homo sapiens
  MOD_RES (8)..(8): Phosphorylated Tyr
IAFWDDVYGFK

SEQ ID NO: 338 - protein, 17 aa, Homo sapiens
  MOD_RES (1)..(1): Phosphorylated Tyr
YPRPLNIIEGPLMNGMK

SEQ ID NO: 339 - protein, 15 aa, Homo sapiens
  MOD_RES (12)..(12): Phosphorylated Tyr
PFFDVWQLRGKYPNR

SEQ ID NO: 340 - protein, 15 aa, Homo sapiens
  MOD_RES (8)..(8): Phosphorylated Tyr
HTIFYKEYPGGPKEL

SEQ ID NO: 341 - protein, 10 aa, Homo sapiens
  MOD_RES (1)..(1): Phosphorylated Tyr
YMVWSDEMVK

SEQ ID NO: 342 - protein, 22 aa, Homo sapiens
  MOD_RES (17)..(17): Phosphorylated Tyr
YSSSPLEWVTLDTNIAYWLHPR

SEQ ID NO: 343 - protein, 34 aa, Homo sapiens
  MOD_RES (27)..(27): Phosphorylated Tyr
SSTVPIPTVNQYLYFLFAPTLIYRDSYPRNPTVR

SEQ ID NO: 344 - protein, 22 aa, Homo sapiens
  MOD_RES (13)..(13): Phosphorylated Tyr
TFVDNMKIYNHSYIYMPAFSMK

SEQ ID NO: 345 - protein, 14 aa, Homo sapiens
  MOD_RES (6)..(6): Phosphorylated Tyr
ILYLFYEDMKKNPK

SEQ ID NO: 346 - protein, 27 aa, Homo sapiens
  MOD_RES (5)..(5): Phosphorylated Tyr
SHLPYRFLPSDLHNGDSKVIYMARNPK

SEQ ID NO: 347 - protein, 27 aa, Homo sapiens
  MOD_RES (21)..(21): Phosphorylated Tyr
SHLPYRFLPSDLHNGDSKVIYMARNPK

SEQ ID NO: 348 - protein, 22 aa, Homo sapiens
  MOD_RES (2)..(2): Phosphorylated Tyr
VYKGEFQLPDFLKEKPQTEQVE

SEQ ID NO: 349 - protein, 26 aa, Homo sapiens
  MOD_RES (4)..(4): Phosphorylated Tyr
PPSYVPVVMSKLSDQMTFMERVKNML

SEQ ID NO: 350 - protein, 17 aa, Homo sapiens
  MOD_RES (6)..(6): Phosphorylated Tyr
FETTKYYITIIDAPGHR

SEQ ID NO: 351 - protein, 21 aa, Homo sapiens
  MOD_RES (2)..(2): Phosphorylated Tyr
VYLTGYNFTLADILLYYGLHR

SEQ ID NO: 352 - protein, 21 aa, Homo sapiens
  MOD_RES (6)..(6): Phosphorylated Tyr
VYLTGYNFTLADILLYYGLHR

SEQ ID NO: 353 - protein, 15 aa, Homo sapiens
  MOD_RES (8)..(8): Phosphorylated Tyr
SEAAYDPYAYPSDYD

SEQ ID NO: 354 - protein, 15 aa, Homo sapiens
  MOD_RES (8)..(8): Phosphorylated Tyr
AAYDPYAYPSDYDMH

SEQ ID NO: 355 - protein, 24 aa, Homo sapiens
  MOD_RES (14)..(14): Phosphorylated Tyr
DMIHIADTKVARRYGDFFIRQIHK

SEQ ID NO: 356 - protein, 8 aa, Homo sapiens
  MOD_RES (8)..(8): Phosphorylated Tyr
QQQSQTAY

SEQ ID NO: 357 - protein, 9 aa, Homo sapiens
  MOD_RES (7)..(7): Phosphorylated Tyr
ARWTETYVR

SEQ ID NO: 358 - protein, 24 aa, Homo sapiens
  MOD_RES (16)..(16): Phosphorylated Tyr
LPKSPPYTAFLGNLPYDVTEESIK

SEQ ID NO: 359 - protein, 26 aa, Homo sapiens
  MOD_RES (3)..(3): Phosphorylated Tyr
TNYNDRYDEIRRHWGGNVLGPKSVAR

SEQ ID NO: 360 - protein, 26 aa, Homo sapiens
  MOD_RES (7)..(7): Phosphorylated Tyr
TNYNDRYDEIRRHWGGNVLGPKSVAR

SEQ ID NO: 361 - protein, 14 aa, Homo sapiens
  MOD_RES (12)..(12): Phosphorylated Tyr
KLTSDDVKEQIYKL

SEQ ID NO: 362 - protein, 12 aa, Homo sapiens
  MOD_RES (9)..(9): Phosphorylated Tyr
GGGHVAQIYAIR

SEQ ID NO: 363 - protein, 7 aa, Homo sapiens
  MOD_RES (3)..(3): Phosphorylated Tyr
ACYGVLR

SEQ ID NO: 364 - protein, 33 aa, Homo sapiens
  MOD_RES (29)..(29): Phosphorylated Tyr
GGRGGDRGGYGGDRSGGGYGGDRSSGGGYSGDR

SEQ ID NO: 365 - protein, 36 aa, Homo sapiens
  MOD_RES (15)..(15): Phosphorylated Tyr
SSGGGYSGDRSGGGYGGDRSGGGYGGDRGGGYGGDR

SEQ ID NO: 366 - protein, 26 aa, Homo sapiens
  MOD_RES (4)..(4): Phosphorylated Tyr
GGGYGGDRGGYGGKMGGRNDYRNDQR

SEQ ID NO: 367 - protein, 26 aa, Homo sapiens
  MOD_RES (4)..(4): Phosphorylated Tyr
GGGYGGDRGGYGGKMGGRNDYRNDQR

SEQ ID NO: 368 - protein, 26 aa, Homo sapiens
  MOD_RES (4)..(4): Phosphorylated Tyr
GGGYGGDRGGYGGKMGGRNDYRNDQR

SEQ ID NO: 369 - protein, 17 aa, Homo sapiens
  MOD_RES (13)..(13): Phosphorylated Tyr
GGYGGDRGGGSGYGGDR

SEQ ID NO: 370 - protein, 25 aa, Homo sapiens
  MOD_RES (10)..(10): Phosphorylated Tyr
DGKMVQKGTYTEFLKSGIDFGSLLK

SEQ ID NO: 371 - protein, 25 aa, Homo sapiens
  MOD_RES (15)..(15): Phosphorylated Tyr
LRRPIGKMTITEQKYEGEYRYVNSR

SEQ ID NO: 372 - protein, 20 aa, Homo sapiens
  MOD_RES (19)..(19): Phosphorylated Tyr
LRRPIGKMTITEQKYEGEYR

SEQ ID NO: 373 - protein, 25 aa, Homo sapiens
  MOD_RES (16)..(16): Phosphorylated Tyr
EQPLDEELKDAFQNAYLELGGLGER

SEQ ID NO: 374 - protein, 16 aa, Homo sapiens
  MOD_RES (7)..(7): Phosphorylated Tyr
VLGFCHYYLPEEQFPK

SEQ ID NO: 375 - protein, 16 aa, Homo sapiens
  MOD_RES (8)..(8): Phosphorylated Tyr
VLGFCHYYLPEEQFPK

SEQ ID NO: 376 - protein, 15 aa, Homo sapiens
  MOD_RES (8)..(8): Phosphorylated Tyr
NQEAVITYQPYLIQP

SEQ ID NO: 377 - protein, 22 aa, Homo sapiens
  MOD_RES (3)..(3): Phosphorylated Tyr
SGYAFSHQEGFGELIMSGKNMR

SEQ ID NO: 378 - protein, 27 aa, Homo sapiens
  MOD_RES (25)..(25): Phosphorylated Tyr
VLVWILTILVILGSLGGTGVLWWLYAK

SEQ ID NO: 379 - protein, 28 aa, Homo sapiens
  MOD_RES (11)..(11): Phosphorylated Tyr
YNDGSPGREFYMDKVLMEFVENSRKAMK

SEQ ID NO: 380 - protein, 11 aa, Homo sapiens
  MOD_RES (3)..(3): Phosphorylated Tyr
GGYLQGNVNGR

SEQ ID NO: 381 - protein, 15 aa, Homo sapiens
  MOD_RES (8)..(8): Phosphorylated Tyr
VGAHAGEYGAEALER

SEQ ID NO: 382 - protein, 15 aa, Homo sapiens
  MOD_RES (8)..(8): Phosphorylated Tyr
IGGHGAEYGAEALER

SEQ ID NO: 383 - protein, 32 aa, Homo sapiens
  MOD_RES (2)..(2): Phosphorylated Tyr
PYILTLGVMMLVGMALYLNGATVVAALIANPR

SEQ ID NO: 384 - protein, 14 aa, Homo sapiens
  MOD_RES (4)..(4): Phosphorylated Tyr
IDHYRHTAAQLGEK

SEQ ID NO: 385 - protein, 11 aa, Homo sapiens
  MOD_RES (6)..(6): Phosphorylated Tyr
DAVVTYTAESK

SEQ ID NO: 386 - protein, 26 aa, Homo sapiens
  MOD_RES (1)..(1): Phosphorylated Tyr
YNVTVIQYIGELLRYLCNSPQKPNDR

SEQ ID NO: 387 - protein, 26 aa, Homo sapiens
  MOD_RES (8)..(8): Phosphorylated Tyr
YNVTVIQYIGELLRYLCNSPQKPNDR

SEQ ID NO: 388 - protein, 25 aa, Homo sapiens
  MOD_RES (12)..(12): Phosphorylated Tyr
FSISPDEDSSSYSSNSDFNYSYPTK

SEQ ID NO: 389 - protein, 25 aa, Homo sapiens
  MOD_RES (20)..(20): Phosphorylated Tyr
FSISPDEDSSSYSSNSDFNYSYPTK

SEQ ID NO: 390 - protein, 15 aa, Homo sapiens
  MOD_RES (8)..(8): Phosphorylated Tyr
HAHPQEVYNEYVPRG

SEQ ID NO: 391 - protein, 33 aa, Homo sapiens
  MOD_RES (6)..(6): Phosphorylated Tyr
NGGGAYLLPYLILLMVIGIPLFFLELSVGQRIR

SEQ ID NO: 392 - protein, 33 aa, Homo sapiens; identical to SEQ ID NO: 391
  MOD_RES (10)..(10): Phosphorylated Tyr

SEQ ID NO: 393 - protein, 20 aa, Homo sapiens
  MOD_RES (2)..(2): Phosphorylated Tyr
PYVEANISHKSHTTIKYFLK

SEQ ID NO: 394 - protein, 20 aa, Homo sapiens
  MOD_RES (17)..(17): Phosphorylated Tyr
PYVEANISHKSHTTIKYFLK

SEQ ID NO: 395 - protein, 29 aa, Homo sapiens
  MOD_RES (10)..(10): Phosphorylated Tyr
NLMANRPAKYKDANIMSPGSSLPSLHVRK

SEQ ID NO: 396 - protein, 21 aa, Homo sapiens
  MOD_RES (16)..(16): Phosphorylated Tyr
LQRSFEEKELASSLAYEERPR

SEQ ID NO: 397 - protein, 30 aa, Homo sapiens
  MOD_RES (24)..(24): Phosphorylated Tyr
EICVVRFTPVTEEDQISYTLLFAYFSSRKR

SEQ ID NO: 398 - protein, 23 aa, Homo sapiens
  MOD_RES (11)..(11): Phosphorylated Tyr
LSPPMLLKEPYKTAVIPINGSPR

SEQ ID NO: 399 - protein, 22 aa, Homo sapiens
  MOD_RES (3)..(3): Phosphorylated Tyr
RKYSFECTDGSSFVDEVEKVVK

SEQ ID NO: 400 - protein, 23 aa, Homo sapiens
  MOD_RES (10)..(10): Phosphorylated Tyr
KNVSINTVTYEWAPPVQNQALAR

SEQ ID NO: 401 - protein, 14 aa, Homo sapiens
  MOD_RES (8)..(8): Phosphorylated Tyr
KKPLDGEYFTLQIR

SEQ ID NO: 402 - protein, 11 aa, Homo sapiens
  MOD_RES (3)..(3): Phosphorylated Tyr
EYYYTKEEQFK

SEQ ID NO: 403 - protein, 11 aa, Homo sapiens
  MOD_RES (4)..(4): Phosphorylated Tyr
EYYYTKEEQFK

SEQ ID NO: 404 - protein, 24 aa, Homo sapiens
  MOD_RES (11)..(11): Phosphorylated Tyr
ATWNDRFSDIYALCVAYPEPLGER

SEQ ID NO: 405 - protein, 20 aa, Homo sapiens
  MOD_RES (2)..(2): Phosphorylated Tyr
FYRTQAPSYLQQNGVQNYMK

SEQ ID NO: 406 - protein, 20 aa, Homo sapiens
  MOD_RES (9)..(9): Phosphorylated Tyr
FYRTQAPSYLQQNGVQNYMK

SEQ ID NO: 407 - protein, 20 aa, Homo sapiens
  MOD_RES (18)..(18): Phosphorylated Tyr
FYRTQAPSYLQQNGVQNYMK

SEQ ID NO: 408 - protein, 34 aa, Homo sapiens
  MOD_RES (10)..(10): Phosphorylated Tyr
QEFVDAYVDYIFNKSVASLFDAFHAGFHKVCGGK

SEQ ID NO: 409 - protein, 15 aa, Homo sapiens
  MOD_RES (8)..(8): Phosphorylated Tyr
AIPSAPLYEEITYSG

SEQ ID NO: 410 - protein, 15 aa, Homo sapiens
  MOD_RES (8)..(8): Phosphorylated Tyr
PLYEEITYSGISDGL

SEQ ID NO: 411 - protein, 21 aa, Homo sapiens
  MOD_RES (19)..(19): Phosphorylated Tyr
VIAGIGLAKKDILGASDPYVR

SEQ ID NO: 412 - protein, 14 aa, Homo sapiens
  MOD_RES (11)..(11): Phosphorylated Tyr
VKGYLRLKMTYLPK

SEQ ID NO: 413 - protein, 13 aa, Homo sapiens
  MOD_RES (11)..(11): Phosphorylated Tyr
IAETFGLQENYIK

SEQ ID NO: 414 - protein, 28 aa, Homo sapiens
  MOD_RES (15)..(15): Phosphorylated Tyr
TNGDGNCLMHATSQYMWGVQDTDLVLRK

SEQ ID NO: 415 - protein, 20 aa, Homo sapiens
  MOD_RES (3)..(3): Phosphorylated Tyr
NYYDLNVLCNFLLENPDYPK

SEQ ID NO: 416 - protein, 20 aa, Homo sapiens
  MOD_RES (18)..(18): Phosphorylated Tyr
NYYDLNVLCNFLLENPDYPK

SEQ ID NO: 417 - protein, 26 aa, Homo sapiens
  MOD_RES (23)..(23): Phosphorylated Tyr
ELADITLDPPPNCSAGPKGDNIYEWR

SEQ ID NO: 418 - protein, 13 aa, Homo sapiens
  MOD_RES (1)..(1): Phosphorylated Tyr
YNLKSPAVKRLMK

SEQ ID NO: 419 - protein, 27 aa, Homo sapiens
  MOD_RES (12)..(12): Phosphorylated Tyr
DIRPGAAFEPTYIYRLLTVNKSSLSEK

SEQ ID NO: 420 - protein, 27 aa, Homo sapiens
  MOD_RES (14)..(14): Phosphorylated Tyr
DIRPGAAFEPTYIYRLLTVNKSSLSEK

SEQ ID NO: 421 - protein, 23 aa, Homo sapiens
  MOD_RES (9)..(9): Phosphorylated Tyr
VADSYSNGYREPPEPDGWAGGLR

SEQ ID NO: 422 - protein, 18 aa, Homo sapiens
  MOD_RES (2)..(2): Phosphorylated Tyr
EYLMRAHFGLPSVEAEDK

SEQ ID NO: 423 - protein, 11 aa, Homo sapiens
  MOD_RES (7)..(7): Phosphorylated Tyr
FLRENPYYDSR

SEQ ID NO: 424 - protein, 32 aa, Homo sapiens
  MOD_RES (2)..(2): Phosphorylated Tyr
CYMYQARDLAAMDKDSFSDPYAIVSFLHQSQK

SEQ ID NO: 425 - protein, 32 aa, Homo sapiens; identical to SEQ ID NO: 424
  MOD_RES (4)..(4): Phosphorylated Tyr

SEQ ID NO: 426 - protein, 32 aa, Homo sapiens; identical to SEQ ID NO: 424
  MOD_RES (21)..(21): Phosphorylated Tyr

SEQ ID NO: 427 - protein, 21 aa, Homo sapiens
  MOD_RES (15)..(15): Phosphorylated Tyr
VRELVDKATNVVMNYSEIESK

SEQ ID NO: 428 - protein, 17 aa, Homo sapiens
  MOD_RES (5)..(5): Phosphorylated Tyr
NKDKYVGVSSDSVGGFR

SEQ ID NO: 429 - protein, 14 aa, Homo sapiens
  MOD_RES (10)..(10): Phosphorylated Tyr
ASTLAMTKEYSFLR

SEQ ID NO: 430 - protein, 12 aa, Homo sapiens
  MOD_RES (3)..(3): Phosphorylated Tyr
NVYATTVGTPYK

SEQ ID NO: 431 - protein, 15 aa, Homo sapiens
  MOD_RES (13)..(13): Phosphorylated Tyr
SSSSQTQPLKVQYQR

SEQ ID NO: 432 - protein, 22 aa, Homo sapiens
  MOD_RES (6)..(6): Phosphorylated Tyr
GREEHYEEEEEEEEDGAAVAEK

SEQ ID NO: 433 - protein, 14 aa, Homo sapiens
  MOD_RES (2)..(2): Phosphorylated Tyr
QYATLDVYNPFETR

SEQ ID NO: 434 - protein, 28 aa, Homo sapiens
  MOD_RES (4)..(4): Phosphorylated Tyr
EAQYNNFSGNSLPEYPTVPSYPGSGQWP

SEQ ID NO: 435 - protein, 30 aa, Homo sapiens
  MOD_RES (26)..(26): Phosphorylated Tyr
QTFLSKLIKSIFISYLENYIEVETGYLKSR

SEQ ID NO: 436 - protein, 21 aa, Homo sapiens
  MOD_RES (16)..(16): Phosphorylated Tyr
YAKLMEWLKSTDYGKYEGLTK

SEQ ID NO: 437 - protein, 22 aa, Homo sapiens
  MOD_RES (12)..(12): Phosphorylated Tyr
VAQGIREEEVSYQLAFNKQELR

SEQ ID NO: 438 - protein, 17 aa, Homo sapiens
  MOD_RES (8)..(8): Phosphorylated Tyr
KFLDTSHYSTAGSSSVR

SEQ ID NO: 439 - protein, 31 aa, Homo sapiens
  MOD_RES (11)..(11): Phosphorylated Tyr
PVVELLSNPDYINQMLLAQLAYREQMNEHHK

SEQ ID NO: 440 - protein, 28 aa, Homo sapiens
  MOD_RES (24)..(24): Phosphorylated Tyr
ASSSSMKIPLNKFPGFAKPGTEQYLLAK

SEQ ID NO: 441 - protein, 11 aa, Homo sapiens
  MOD_RES (8)..(8): Phosphorylated Tyr
NILSSADYVER

SEQ ID NO: 442 - protein, 9 aa, Homo sapiens
  MOD_RES (9)..(9): Phosphorylated Tyr
KTAGLSDLY

SEQ ID NO: 443 - protein, 21 aa, Homo sapiens
  MOD_RES (3)..(3): Phosphorylated Tyr
EKYDNMAELFAVVKTMQALEK